SEQUENCE LISTING (4 sequences)

SEQ ID NO: 1 - protein, 15 aa, Artificial Sequence
  Synthetic polypeptide
GPLGLAGGWGERDGS

SEQ ID NO: 2 - protein, 6 aa, Artificial Sequence
  Synthetic polypeptide
PLGLAG

SEQ ID NO: 3 - protein, 11 aa, Artificial Sequence
  Synthetic polypeptide
LAGGWGERDGS

SEQ ID NO: 4 - protein, 12 aa, Artificial Sequence
  Synthetic polypeptide
GPLGLAGGERDG

(19) United States
(12) Patent Application Publication
(10) Pub. No.: US 2018/0092845 A1
(43) Pub. Date: Apr. 5, 2018

(54) ENZYME-RESPONSIVE NANOPARTICLES

(71) Applicant: THE REGENTS OF THE UNIVERSITY OF CALIFORNIA, Oakland, CA (US)

(72) Inventors: Nathan C. Gianneschi, San Diego, CA (US); Karen L. Christman, San Diego, CA (US)

(21) Appl. No.: 15/788,692

(22) Filed: Oct. 19, 2017

Related U.S. Application Data
(63) Continuation of application No. PCT/US2016/028711, filed on Apr. 21, 2016.
(60) Provisional application No. 62/150,551, filed on Apr. 21, 2015.

(51) Int. Cl. A61K 9/107; C08G 61/08; A61K 31/787; A61P 9/10; A61P 9/04

(52) U.S. Cl. CPC A61K 9/1075; C08G 61/08; A61K 31/787; A61P 9/10; C08G 2261/1426; C08G 2261/126; C08G 2261/3324; C08G 2261/149; C08G 2261/148; A61P 9/04

(57) ABSTRACT

Described herein, inter alia, are compositions and methods for using block copolymers and polymeric micelles for treating myocardial infarction, cardiomyopathy, or heart failure in a subject in need thereof.

### CROSS-REFERENCES TO RELATED APPLICATIONS

This application claims the benefit of U.S. Provisional Application No. 62/150,551, filed Apr. 21, 2015, which is incorporated herein by reference in entirety and for all purposes.

### STATEMENT AS TO RIGHTS TO INVENTIONS MADE UNDER FEDERALLY SPONSORED RESEARCH AND DEVELOPMENT

This invention was made with government support under grant no. HL117326 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 48537-549001WO_ST25.txt, created Apr. 15, 2016, 1,157 bytes, machine format IBM-PC, MS Windows operating system, is hereby incorporated by reference.

### BACKGROUND

Each year approximately three quarters of a million Americans will have a new myocardial infarction (MI), and approximately half a million will have a recurrent MI. Approximately 37% of these patients will die from the MI within one year, and of those who do survive, two-thirds will not make a complete recovery, leading to an extremely large patient population that progresses to heart failure. These statistics necessitate the development of new innovative therapies for MI. Currently, early treatment of MI is not possible because it would require direct injection of materials into inflamed tissue constituting an unacceptable risk. Disclosed herein are solutions to these and other problems in the art.

### BRIEF SUMMARY OF THE INVENTION

In one aspect, provided herein is a block copolymer including a first block of hydrophobic polymerized monomers and a second block of hydrophilic polymerized monomers, wherein the first block of hydrophobic polymerized monomers include a hydrophobic moiety covalently attached to each first block monomer backbone moiety within the first block of hydrophobic polymerized monomers, wherein each hydrophobic moiety is optionally different; and the second block of hydrophilic polymerized monomers include a hydrophilic moiety covalently attached to each second block monomer backbone moiety within the second block of hydrophilic polymerized monomers, wherein each hydrophilic moiety is optionally different, and wherein at least one of the hydrophilic moieties includes an inflammatory protease cleavable amino acid sequence.

In one aspect, provided herein is a block copolymer including a first block of hydrophobic polymerized monomers and a second block of hydrophilic polymerized monomers, wherein: (i) the first block of hydrophobic polymerized monomers includes a hydrophobic moiety covalently attached to each first block monomer backbone moiety within the first block of hydrophobic polymerized monomers, wherein each hydrophobic moiety is optionally different; and (ii) the second block of hydrophilic polymerized monomers includes a hydrophilic moiety covalently attached to each second block monomer backbone moiety within the second block of hydrophilic polymerized monomers, wherein each hydrophilic moiety is optionally different, and wherein at least one of the hydrophilic moieties includes an MMP-9 or MMP-2 cleavable amino acid sequence.

In another aspect, provided herein is an aqueous pharmaceutical composition including a polymeric micelle as described herein and a pharmaceutically acceptable excipient.

In another aspect, provided herein is a method of treating myocardial infarction in a subject in need thereof, the method including administering to the subject a therapeutically effective amount of the block copolymer, the polymeric micelle, or the pharmaceutical composition as described herein.

In another aspect, provided herein is a method of forming a polymeric aggregate, the method including: contacting the block copolymer as described herein with an inflammatory protease; and allowing the inflammatory protease to cleave the inflammatory protease cleavable amino acid sequence, thereby forming a polymeric aggregate.

In another aspect, provided herein is a method of forming a polymeric aggregate, the method including: contacting the block copolymer as described herein with an MMP-2 or MMP-9; and allowing the MMP-2 or MMP-9 to cleave the MMP-2 or MMP-9 cleavable amino acid sequence, respectively, thereby forming a polymeric aggregate.

In another aspect, provided herein is a polymeric micelle including a plurality of the block copolymers as described herein, the polymeric micelle including a hydrophobic core including the first block of hydrophobic polymerized monomers and a hydrophilic shell including the second block of hydrophilic polymerized monomers. In embodiments, the polymeric micelle is a nanoparticle (e.g., a spherical nanoparticle).

In another aspect, provided herein is a method of treating heart failure in a subject in need thereof, the method including administering to the subject a therapeutically effective amount of the block copolymer, the polymeric micelle, or the pharmaceutical composition as described herein.

In another aspect, provided herein is a method of performing cardiovascular surgery in a subject in need thereof, the method including performing a surgical procedure; and administering to the subject a therapeutically effective amount of the block copolymer, the polymeric micelle, or the pharmaceutical composition as described herein.

### BRIEF DESCRIPTION OF THE DRAWINGS

FIGS. 1A-1E. Responsive nanoparticles target, accumulate, and are retained within an acute myocardial infarction. (FIG. 1A) Diagram of a dye-labeled brush peptide-polymer amphiphile (PPA) containing an MMP-2 and MMP-9 specific recognition sequence, shown underlined. PPAs self-assemble into nanoparticles through hydrophobic-hydrophilic interactions when dialyzed into aqueous buffer. Sequence legend: GPLGLAGGWGERDGS (FIG. 1B) Schematic of nanoparticles freely circulating in the bloodstream (not to scale). (FIG. 1C) Nanoparticles enter the infarct tissue through the leaky acute MI vasculature, and upregulated MMPs within the infarct induce the formation of an aggregate-like scaffold. FIG. 1 (D) In vitro, nanoparticles are monodisperse micelles with diameters of 15-20 nm, and (FIG. 1E) upon activation, an aggregate-like scaffold (0.7-14 μm) is observed. Scale bar: 100 nm.

FIGS. 2A-2C. Retention of MMP pretreated responsive nanoparticles and clearance of nonresponsive nanoparticles in healthy myocardium. MMP pretreated particles were injected into healthy rat myocardium (FIG. 2A) and tissue was evaluated 1 minute, 1 hour, 1 day, 2 days, and 7 days post-injection for presence of the fluorescent aggregates (FIGS. 2B-2C). Rhodamine labeled, responsive particles (FIGS. 2B-2C, top row) were observed at the site of injection up to 7 days post-injection, while the nonresponsive particles (FIGS. 2B-2C, bottom row) were cleared after 1 hour. Scale bar: 1 mm (FIG. 2A); 50 μm (FIGS. 2B-2C).

FIG. 3. Retention of responsive nanoparticles upon localized delivery. Particles were injected intramyocardially 7 days post-MI and assessed 6 days post-injection. H&E images display the infarct area in FIG. 3 (left), and neighboring fluorescent sections are shown in FIG. 3 (middle). Particles and myocardium, which was labeled with an anti-α-actinin antibody, is shown. Selected regions from FIG. 3 (middle) highlighted with white outline were magnified to highlight particle aggregation in FIG. 3 (right). Scale bar: 100 μm.

FIG. 4. Retention of IV delivered nanoparticles with the infarct. H&E images are shown in FIG. 4 (left), and neighboring fluorescent sections are shown in FIG. 4 (middle). Selected magnified regions from FIG. 4B (middle) highlighted in with white outline are shown in FIG. 4 (left). In the absence of an infarct, IV injected responsive particles do not accumulate in healthy myocardium. Very few nonresponsive particles (NR) particles, which were IV injected 24 hours post-MI, were observed in the infarct 2 days post-injection. In contrast, aggregates of responsive particles were observed in the infarct 2, 7, 14, or 28 days post-injection. Scale bar: 100 μm.

FIGS. 5A-5D. Preparation of fluorescent MMP-9 responsive nanoparticles. Block copolymers were synthesized with a hydrophobic phenyl-moiety and a conjugatable N-hydroxy succinimide-ester prepared via ring-opening metathesis polymerization. [8] The living polymer was modified either with a fluorescein or rhodamine-labeled termination agent and further modified with a peptide sequence consisting of either L-amino acids as a cleavable substrate or D-amino acids as a nonresponsive substrate. Peptide polymer amphiphiles (PPA) were combined in a 1:1 molar ratio and dialyzed into 1×DPBS to create micellar nanoparticles.

FIG. 6. SEC-MALS intensity plot of polymers 1₂₀, 1₂₀-b-2₅, 1₂₀-b-2₅-Dye, and peptide conjugated 1₂₀-b-2₅-Dye PPA. Light scattering (LS) and differential refractive index (RI) traces are show as solid and dashed lines, respectively.

FIGS. 7A-7C. Spectroscopic analysis of nanoparticles in 8:1 DMSO: 1×DPBS. (FIG. 7A) Absorbance spectrum of nanoparticles labeled with fluorescein (λₘₐₓ=516 nm) and rhodamine (λₘₐₓ=565 nm). (FIG. 7B) Absorbance calibration curves as a function of PPA, or total dye, concentration (μM). Trendline slopes 3.44851×10⁻³ (Fl-PPA) and 4.74505×10⁻³. R₂ values 0.99803 (Fl-PPA) and 0.99995 (Rho-PPA). (n=2) (FIG. 7C) Normalized fluorescence intensity of excitation and emission scans for nanoparticles.

FIGS. 8A-8B. DLS spectra of responsive (FIG. 8A) and nonresponsive (FIG. 8B) particle size distributions with respect to % mass vs diameter (nm) before and after 24 hr MMP-9 treatment. Inset figures show treated particle solutions just before DLS measurement.

FIG. 9. Dry state TEM micrographs of nanoparticles before and after 24 hr of MMP-9 treatment. (FIG. 9, top row) responsive particles before MMP-9 treatment, (FIG. 9, second row) responsive particles after MMP-9 treatment, (FIG. 9, third row) nonresponsive nanoparticles before MMP-9 treatment, (FIG. 9, last row) nonresponsive nanoparticles after MMP-9 treatment. Images are provided with scale bars from left to right measuring 200 nm, 100 nm, and 50 nm, respectively.

FIGS. 10A-10C. Spectroscopic and Spectrometric Analysis of MMP-9 Treated Nanoparticles. (FIG. 10A) Analytical HPLC of responsive and nonresponsive nanoparticles treated with either active or denatured MMP-9. A linear elution gradient of 0-30% buffer B over 30 min, then 30-50% buffer B over 10 min measured at 214 nm (flow rate: 1 mL/min) was used to resolve peaks. The cleavage fragment for enzyme treated responsive nanoparticles (indicated by the asterisks) at Rt=19 min (16% buffer B) was measured by (FIG. 10B) ESI MS using the negative ion mode. Observed: [M−H]⁻ 1102.44 m/z. Calculated: [M−H]⁻ 1102.15 m/z. (FIG. 10C) The cleaved peptide product, LAGGWGERDGS, (SEQ NO:3) chemical structure is indicated.

FIG. 11. Effect of reduced permeability on nanoparticle delivery. Responsive nanoparticles were injected IV 30 days post-MI, and the animals survived for 2 days post-injection. H&E image is shown in FIG. 11 (left), and the neighboring fluorescent section, with particles and the myocardium shown in FIG. 11 (middle). Selected magnified region from FIG. 11 (middle) with a white outline is shown in FIG. 11 (right). Scale bar: 100 μm.

FIG. 12. Biocompatibility of responsive nanoparticles. A CD68 stain was used to evaluate the presence of macrophages, which are stained dark brown. CD68-positive cells were observed in both the infarct control animal 29 days post-MI (FIG. 12 (left)), and infarct with responsive particles 28 days-post-injection (FIG. 12 (right)). Scale: 100 μm.

FIG. 13. Biodistribution of responsive nanoparticles. H&E stained images of satellite organs of animals that received responsive particles 24 hours post-MI over 2, 7, 14, and 28 days post-injection. Respective organs from infarct only animals 3 days (C3) and 29 days (C29) post-MI were used as controls. Scale: 100 μm.

FIG. 14. Biodistribution of responsive nanoparticles. Fluorescent microscopy images of satellite organs from animals that received responsive particles 24 hours post-MI over 2, 7, 14, and 28 days post-injection. Images were captured in the red channel. Scale: 20 μm.

FIGS. 15A-15D. Synthesis of drug-loaded, enzyme responsive micellar nanoparticles. FIG. 15A: Polymerization scheme of enzyme-responsive, paclitaxel-conjugated diblock copolymers. FIG. 15B: Nanoparticles assemble, and subsequently change morphology in response to MMP. FIG. 15C: TEM image (dry state, negative uranyl acetate stain) of NP_{L} before and FIG. 15D after exposure to MMP, demonstrating enzyme-induced morphology change.

FIGS. 16A-16C. FIG. 16A: Maximum Tolerated Dose (MTD) of IV injection of NP_{L} and clinically formulated PTX. Note: LD50 of clinical PTX is 30 mg/kg and MTD is 15 mg/kg. FIG. 16B: Comparison of NP_{L} to NP_{D} following IT injection. NP_{L} effectively inhibits tumor growth up to 12 days post-injection, whereas NP_{D} has no observable effect. Note: clinical paclitaxel cannot be IT injected without severe adverse effects (ulceration). FIG. 16C: Comparison of NP_{L} and NP_{D} vs. clinical paclitaxel following IV injection.

FIG. 17. Ex vivo tissue analysis. Fluorescence imaging of NP_{L} (left), NP_{D} (middle) and saline (right) cohorts at 14 days post-IV injection. Organs were imaged immediately after excision, and include tumor, liver, spleen, kidneys, heart, and lung, from top to bottom in each panel.

FIG. 18. HPLC Analysis of Nanoparticles Treated with MMP. The elution time of the peptide cleavage fragment (LAGGERDG) is 14 minutes (absorbance at 214 nm), and MW confirmed by ESI-MS (inset, negative ion mode). Note that only this fragment only occurs in NP_{L}, demonstrating that NP_{D} is not being cleaved by MMP.

FIGS. 19A-19D. Nanoparticle Aggregation Analysis Post-MMP Introduction. Upon exposure to MMP, NP_{L} undergoes a drastic morphology change, but NP_{D} does not. The image on the upper left (FIG. 19A) is the negative stain TEM of NP_{D} prior to enzyme treatment. The image on the upper right (FIG. 19B) is the negative stain TEM of NP_{D} after treatment with MMP. Unlike for that of NP_{L}, there is no evidence of morphology change in NP_{D} by neither TEM nor DLS.

FIG. 20. Biodistribution of NP_{L} in various organs following IV or IT injection. Biodistribution of NP_{L} was assessed by measuring fluorescence in various homogenized tissues (tumor, liver, spleen, and kidney) at 48 hours following IV injection or IT injection. Using tissue-specific calibration curves, fluorescence counts were converted to polymer concentrations. These values were then normalized by the injected dose and animal weight to calculate standardized uptake values. The data suggests that the mode of clearance of these materials is through RES-associated organs, which is typical of nanoparticle formulations. Further, the highest accumulation of NP_{L} following IT injection is in the tumor tissue, which suggests that although the material is being cleared from the tissue, there is still presence of material at 2 days post-injection.

FIG. 21. HPLC Analysis of NP_{L} to monitor for PTX release. The MMP-degraded NP_{L} system was analyzed for evidence of PTX hydrolysis off the polymer backbone. As a control, PTX was subjected to the same MMP cleavage conditions (peaks appearing between 27 and 32 minutes are due to the enzyme). ESI-MS of the peak eluting at 26 minutes was used to confirm peak identity (inset, negative ion mode).

FIGS. 22A-22C. SLS Characterization of the copolymers.

FIGS. 23A-23B. Live-animal optical imaging of NP_{L} and NP_{D}. Animals IT injected with either NP_{L} or NP_{D} were monitored via live-animal optical imaging (λₑₓ=470 nm and λₑₘ=590 nm) for presence of FRET signal. In animals treated with NP_{L}, FRET is observable up to three days post-injection, indicative of retained material. In contrast, the FRET signal in animals treated with NP_{D} drops below the threshold after 3 hours, suggesting these materials are being cleared from the tissue at a faster rate than those of NP_{L}, presumably due to lack of morphology change.

### DETAILED DESCRIPTION

Herein we describe, inter alia, a method for targeting and retaining intravenous (IV) injected polymeric micelles to the site of acute myocardial infarction (MI). In embodiments, block copolymers are prepared and assembled into polymeric micellar nanoparticles. The resulting nanoparticles may respond to matrix metalloproteinases such as MMP-2 and MMP-9 which are upregulated in heart tissue post-myocardial infarction. The polymeric micelles may undergo a morphological transition from spherical-shaped, discrete materials to network-like assemblies when acted upon by MMPs.

Further disclosed herein, inter alia, are self-assembling materials programmed to form a healing scaffold in damaged heart tissue immediately following MI, including the negative left ventricular (LV) remodeling that occurs post-MI and results in heart failure. Accordingly, there are disclosed new methods and materials for the design of treatments for healing heart tissue post-MI. In embodiments, this approach uses an enzyme activated morphology switch that creates aggregate assemblies of nanoparticles that are retained long term at the site of myocardial infarction. In embodiments, materials are disclosed to be injected intravenously rather than directly into heart tissue. Such materials can circulate and assemble into the healing scaffold in response to inflammatory enzymes present in damaged heart tissue (e.g., matrix metalloproteinases, MMPs).

In embodiments, the polymeric micelles (e.g. nanoparticles) may include brush peptide-polymer amphiphiles (PPAs) that have been prepared and assembled into spherical micellar nanoparticles. In embodiments, the polymeric micelles (e.g. nanoparticles) may include peptide-polymer amphiphiles that have been prepared and assembled into spherical micellar nanoparticles. In embodiments, the resulting polymeric micelles (e.g. nanoparticles) respond (e.g., react) to matrix metalloproteinases (MMP-2 and MMP-9) that are unregulated in heart tissue post-myocardial infarction. In embodiments, the polymeric micelles respond to an inflammatory protease. The polymeric micelles (e.g. nanoparticles) may undergo a morphological transition from spherical-shaped, discrete materials to network-like assemblies when acted upon by MMPs. In embodiments, a 15-20 nm polymeric micelle is injected intravenously, and undergoes reaction with MMPs in the heart after MI, with the resulting assemblies remaining within the infarct (e.g., site of initial heart tissue damage) for up to 28 days.

In embodiments, the polymeric micelles (e.g. nanoparticles) provided herein are delivered to a patient's heart (e.g., via intravenous, intracoronary, or intramyocardial delivery) within the first day post-MI. In embodiments, the extracellular matrix framework is stabilized as a result of delivery of the polymeric micelles (e.g. nanoparticles), which may additionally function to facilitate cell infiltration to alter the typical infarct process. Thus, provided herein, inter alia, are polymeric micelles (e.g. composed of novel copolymers) capable of forming a scaffold material architecture (e.g., polymeric aggregate) in response to MMPs in damaged heart tissue. In embodiments, provided herein are autonomously assembling scaffolds (e.g. nanomaterials), which can be delivered via IV injection, target the area of acute MI, prevent and/or slow negative left ventricular remodeling, and improve cardiac function post-MI.

In embodiments, the polymeric micelles (e.g. nanoparticles) provided herein function as an infarct-specific probe and/or therapeutic biomaterial that can remain in the infarcted tissue throughout an effective therapeutic window. The polymeric micelles (e.g. nanoparticles) may possess retention times on the order of one week to months. In embodiments, the polymeric micelles are used to overcome rapid infarct tissue clearance and drastically enhancing biomaterial retention within the infarct tissue as compared with current approaches, which are cleared from the tissue in a matter of days regardless of their active targeting mechanism(s). In embodiments, the system provided herein utilizes a stimulus-triggered morphology switch that converts discrete nanoscale micelles into micron-scale aggregated scaffolds; this substantial change in size at the target tissue, through an active targeting mechanism, may increase residence time in the infarct.

The approach provided herein may enable IV delivering of a biomaterial and therapeutic to the heart. The approach provided herein, therefore, may enable treatment during the very early stages of a MI, which can have significant clinical advantages.

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene (e.g., contains an unsaturated bond).

The term “heteroalkyl,” by itself or in combination with another term, means, unless otherwise stated, a stable straight or branched chain, or combinations thereof, including at least one carbon atom and at least one heteroatom (e.g., O, N, P, Si, or S), and wherein the nitrogen and sulfur atoms may optionally be oxidized, and the nitrogen heteroatom may optionally be quaternized. The heteroatom(s) (e.g., O, N, P, S, or Si) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

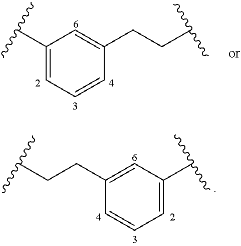 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SW, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R″′)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:

(A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
(i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(ii) alkyl, heteroalkyl, cycloalkyl, heterocycloalkyl, aryl, heteroaryl, substituted with at least one substituent selected from:
(a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those that are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

A “detectable moiety” as used herein refers to a moiety that can be covalently or noncovalently attached to a compound or biomolecule that can be detected, for instance, using techniques known in the art. In embodiments, the detectable moiety is covalently attached. The detectable moiety may provide for imaging of the attached compound or biomolecule. The detectable moiety may indicate the contacting between two compounds. Exemplary detectable moieties are fluorophores, antibodies, reactive dyes, radio-labeled moieties, magnetic contrast agents, and quantum dots. Exemplary fluorophores include fluorescein, rhodamine, GFP, coumarin, FITC, Alexa fluor, Cy3, Cy5, BODIPY, and cyanine dyes. Exemplary radionuclides include Fluorine-18, Gallium-68, and Copper-64. Exemplary magnetic contrast agents include gadolinium, iron oxide and iron platinum, and manganese.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Prodrugs of the compounds described herein may be converted in vivo after administration. Additionally, prodrugs can be converted to the compounds of the present invention by chemical or biochemical methods in an ex vivo environment, such as, for example, when contacted with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The terms “polypeptide,” “peptide” and “protein” are used interchangeably herein to refer to a polymer of amino acid residues, wherein the polymer may optionally be conjugated to a moiety that does not consist of amino acids (e.g., a block copolymer). The terms apply to amino acid polymers in which one or more amino acid residue is an artificial chemical mimetic of a corresponding naturally occurring amino acid, as well as to naturally occurring amino acid polymers and non-naturally occurring amino acid polymer.

A polypeptide, or a cell is “recombinant” when it is artificial or engineered, or derived from or contains an artificial or engineered protein or nucleic acid (e.g. non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “treating”, or “treatment” refers to any indicia of success in the therapy or amelioration of an injury, disease, pathology or condition (e.g., myocardial infarction, cardiomyopathy, heart failure), including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease (e.g., myocardial infarction, cardiomyopathy, heart failure). In embodiments, treating is preventing. In embodiments, treating does not include preventing.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of an injury or disease (e.g., myocardial infarction, cardiomyopathy, heart failure), which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms (e.g., myocardial infarction, cardiomyopathy, heart failure). The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, severity of symptom or symptoms of an injury or disease (e.g., infarct size and location), and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, intracoronary, intramyocardial, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal) compatible with the preparation. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

“Co-administer” it is meant that a composition described herein is administered at the same time, just prior to, or just after the administration of one or more additional therapies. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered transdermally, by a topical route, or formulated as applicator sticks, solutions, suspensions, emulsions, gels, creams, ointments, pastes, jellies, paints, powders, and aerosols.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

“Disease” or “condition” refer to a state of being or health status of a patient or subject capable of being treated with the compounds or methods provided herein. In some embodiments, the disease is a disease related to (e.g. caused by) heart muscle damage (e.g. myocardial infarction, cardiomyopathy, heart failure). In some instances, “disease” or “condition” refers to myocardial infarction, cardiomyopathy, or heart failure. In some embodiments, the disease is heart muscle damage. In some embodiments, the disease is myocardial infarction. In some embodiments, the disease is heart failure. In some embodiments, the disease is cardiomyopathy. In some embodiments, the disease is hypertrophic cardiomyopathy. In some embodiments, the disease is restrictive cardiomyopathy. In some embodiments, the disease is dilated cardiomyopathy. In some embodiments, the disease is dilated congestive cardiomyopathy. In some embodiments, the disease is congestive cardiomyopathy. In some embodiments, the disease is cardiomyopathy associated with or caused by hypertension, heart valve disease, myocardial ischemia, myocardial inflammation, myocardial infarction, heart failure, pulmonary hypertension, myocardial stunning, myocardial hibernation, cardiac surgery, or coronary intervention. In some embodiments, the disease is heart failure associated with or caused by cardiomyopathy. In some embodiments, the disease is heart failure associated with or caused by cardiomyopathy (e.g. associated with or caused by hypertension, heart valve disease, myocardial ischemia, myocardial inflammation, myocardial infarction, pulmonary hypertension, myocardial stunning, myocardial hibernation, cardiac surgery, or coronary intervention). In some embodiments, the disease is heart failure associated with or caused by idiopathic cardiomyopathy. In some embodiments, the disease is cardiomyopathy associated with or caused by hypertension, heart valve disease, myocardial ischemia, myocardial inflammation, heart failure, pulmonary hypertension, myocardial stunning, myocardial hibernation, cardiac surgery, or coronary intervention. In some embodiments, the disease is heart failure associated with or caused by cardiomyopathy (e.g. associated with or caused by hypertension, heart valve disease, myocardial ischemia, myocardial inflammation, pulmonary hypertension, myocardial stunning, myocardial hibernation, cardiac surgery, or coronary intervention). In some embodiments, the disease is cardiomyopathy associated with or caused by hypertension, heart valve disease, myocardial inflammation, heart failure, pulmonary hypertension, myocardial stunning, myocardial hibernation, cardiac surgery, or coronary intervention. In some embodiments, the disease is heart failure associated with or caused by cardiomyopathy (e.g. associated with or caused by hypertension, heart valve disease, myocardial inflammation, pulmonary hypertension, myocardial stunning, myocardial hibernation, cardiac surgery, or coronary intervention). In embodiments the disease is myocarditis.

As used herein, the term “cardiomyopathy” refers to a disease or condition affecting the heart, wherein a heart muscle (e.g., cell of the heart muscle) is damaged or the function of a heart muscle (e.g., cell of the heart muscle) is impaired (e.g., relative to a healthy fully functioning heart, heart muscle, of heart muscle cell). An exemplary cardiomyopathy that may be treated with a compound or method provided herein include heart muscle damage, alcoholic cardiomyopathy, arrhythmogenic right ventricular cardiomyopathy, restrictive cardiomyopathy, noncompaction cardiomyopathy, heart failure, (congestive) heart failure, hypertensive cardiomyopathy, cardiomyopathy associated with cardiac surgery, cardiomyopathy associated with coronary intervention or myocardial infarction, cardiomyopathy caused by genetic changes in cardiac proteins, cardiomyopathy associated with genetic mutations in one or more cardiac proteins, and cardiomyopathy associated with aberrant expression or function of one or more cardiac proteins. In some embodiments, treating a cardiomyopathy includes treating a condition or symptom caused by a cardiomyopathy. In some embodiments, cardiomyopathy is caused by another disease (e.g., a cardiovascular disease) and treatment of cardiomyopathy includes treating the causative disease (e.g. cardiovascular disease) of the cardiomyopathy. In some embodiments, the cardiomyopathy is dilated cardiomyopathy. In some embodiments, the cardiomyopathy is hypertrophic cardiomyopathy. In some embodiments, the cardiomyopathy is hypertrophic, restrictive, or dilated.

The term “myocardial infarction” is used in accordance with its ordinary meaning and refers to heart tissue damage and or myocardial cell death resulting from a decrease in blood flow. In embodiments, myocardial infarction is caused by prolonged ischemia. A patient having symptoms of myocardial infarction can be diagnosed by clinical features, such as electrocardiographic (ECG) findings, elevated values of biochemical markers (e.g., proteins) of myocardial necrosis (e.g., cardiac troponins), and/or by imaging. Symptoms associated with myocardial infarction include myocardial ischemia, tachycardia arrhythmia, bradycardia arrhythmia, arrhythmia, heart failure, heart tissue damage, or renal failure.

The terms “heart failure” or “congestive heart failure” are used in accordance with their ordinary meaning and refers to a condition affecting the heart, wherein a heart muscle (e.g., cell of the heart muscle) is damaged or the function of a heart muscle (e.g., cell of the heart muscle) is impaired (e.g., relative to a healthy fully functioning heart, heart muscle, of heart muscle cell). In embodiments, heart failure is a condition affecting the heart, wherein the heart exhibits reduced blood flow.

The term “inflammatory protease cleavable amino acid sequence” refers to an amino acid sequence that is cleavable (e.g. specifically cleavable) by an inflammatory protease. The term “inflammatory protease” refers to a proteolytic enzyme found at or around the site of inflammation. Inflammatory proteases include proteases that are upregulated following inflammation or tissue damage, proteases that are specifically directed to the site of inflammation through biological processes, or proteases that perform specific biological functions related to inflammation at the site of inflammation. Inflammatory proteases may be expressed at and/or targeted to the site of inflammation. In embodiments, the inflammatory protease is found at the site of inflammation. Non-limiting examples of an inflammatory protease include a membrane bound MMP (e.g., MT1-MMP, MT2-MMP, MT3-MMP, MT4-MMP, MT5-MMP, MT6-MMP), serine protease (e.g., plasmin, cathepsin G), lysosomal cysteine protease (cathepsin B), tryptase, chymase, collagenase (e.g., MMP-1, MMP-8, MMP-13), gelatinase (MMP-2, MMP-9), stromelysin (e.g., MMP-3), or membrane type (e.g., MMP-14). In embodiments, the inflammatory protease is MMP-1, MMP-2, MMP-3, MMP-7, MMP-9, or MMP-14.

The term “cardiovascular inflammatory protease cleavable amino acid sequence” refers to an amino acid sequence that is cleavable by a cardiovascular inflammatory protease. The term “cardiovascular inflammatory protease” refers to an inflammatory protease upregulated following inflammation or tissue damage at a cardiovascular tissue site of inflammation (e.g. cardiac tissue, cardiovascular arteries, etc.), proteases that are specifically directed to the cardiovascular tissue site of inflammation through biological processes, or proteases that perform specific biological functions related to inflammation at the cardiovascular tissue site of inflammation. Cardiovascular inflammatory proteases may be expressed at and/or targeted to the site of cardiovascular inflammation. In embodiments, the cardiovascular inflammatory protease is found at the site of cardiovascular inflammation (e.g., inflammation at cardiac tissue resulting from stent placement, angioplasty, or surgical procedure). Non-limiting examples of a cardiovascular inflammatory protease include membrane bound MMP (e.g., MT1-MMP, MT2-MMP, MT3-MMP, MT4-MMP, MT5-MMP, MT6-MMP), serine protease (e.g., plasmin, cathepsin G), lysosomal cysteine protease (cathepsin B), tryptase, chymase, collagenase (e.g., MMP-1, MMP-8, MMP-13), gelatinase (MMP-2, MMP-9), stromelysin (e.g., MMP-3), or membrane type (e.g., MMP-14). In embodiments, the cardiovascular inflammatory protease is MMP-1, MMP-2, MMP-3, MMP-7, MMP-9, or MMP-14.

The term “myocardial inflammatory protease cleavable amino acid sequence” refers to an amino acid sequence that is cleavable by a myocardial inflammatory protease. The term “myocardial inflammatory protease” refers to an inflammatory protease upregulated following inflammation or tissue damage at a myocardial tissue site of inflammation, proteases that are specifically directed to the myocardial tissue site of inflammation through biological processes, or proteases that perform specific biological functions related to inflammation at the myocardial tissue site of inflammation. In embodiments, the myocardial inflammatory protease cleavable amino acid sequence is SEQ ID NO:1. In embodiments, the myocardial inflammatory protease cleavable amino acid sequence is SEQ ID NO:2. In embodiments, the myocardial inflammatory protease cleavable amino acid sequence is SEQ ID NO:3. In embodiments, the myocardial inflammatory protease cleavable amino acid sequence is SEQ ID NO:4. Myocardial inflammatory proteases may be expressed at and/or targeted to the site of myocardial inflammation. In embodiments, the myocardial inflammatory protease is found at the site of myocardial inflammation (e.g., inflammation at myocardial tissue resulting from myocardial infarction, cardiomyopathy, heart failure, or a surgical procedure). Non-limiting examples of a myocardial inflammatory protease include membrane bound matrix metalloproteinase (MMP) (e.g., MT1-MMP, MT2-MMP, MT3-MMP, MT4-MMP, MT5-MMP, MT6-MMP), serine protease (e.g., plasmin, cathepsin G), lysosomal cysteine protease (cathepsin B), tryptase, chymase, collagenase (e.g., MMP-1, MMP-8, MMP-13), gelatinase (MMP-2, MMP-9), stromelysin (e.g., MMP-3), or membrane type (e.g., MMP-14). In embodiments, the myocardial protease is MMP-1, MMP-2, MMP-3, MMP-7, MMP-9, or MMP-14.

The term “MMP” refers to a matrix metalloprotease. The terms “matrix metalloproteinase-2”, “MMP2”, and “MMP-2” are used according to the plain and ordinary meaning in the art and refer to an enzyme by the same name involved in the breakdown of extracellular matrix (e.g. 72 kDa type IV collagenase also known as matrix metalloproteinase-2 (MMP-2) and gelatinase A is an enzyme that in humans is encoded by the MMP2 gene). The term “MMP-2” may refer to the nucleotide sequence or protein sequence of human MMP-2 (e.g., Entrez 4313, Uniprot P08253, RefSeq NM_001127891, or RefSeq NP_001121363). The term “MMP-2” includes both the wild-type form of the nucleotide sequences or proteins as well as any mutants thereof. In some embodiments, “MMP-2” is wild-type MMP-2 receptor. In some embodiments, “MMP-2” is one or more mutant forms. The term “MMP-2” XYZ refers to a nucleotide sequence or protein of a mutant MMP-2 wherein the Y numbered amino acid of MMP-2 that normally has an X amino acid in the wildtype, instead has a Z amino acid in the mutant. In embodiments, an MMP-2 is the human MMP-2. In embodiments, the MMP-2 has the nucleotide sequence corresponding to reference number GI:700274109. In embodiments, the MMP-2 has the nucleotide sequence corresponding to RefSeq NM_001127891.2. In embodiments, the MMP-2 has the protein sequence corresponding to reference number GI:189217853. In embodiments, the MMP-2 has the protein sequence corresponding to RefSeq NP_001121363.1.

The terms “matrix metalloproteinase-9”, “MMP9”, and “MMP-9” are used according to the plain and ordinary meaning in the art and refer to an enzyme by the same name involved in the breakdown of extracellular matrix (e.g. a 92 kDa type IV collagenase, 92 kDa gelatinase or gelatinase B (GELB). MMP9 may function as a matrixin, a class of enzymes that belong to the zinc-metalloproteinases family involved in the degradation of the extracellular matrix. The term “MMP-9” may refer to the nucleotide sequence or protein sequence of human MMP-9 (e.g., Entrez 4318, Uniprot P14780, RefSeq NM_004994, or RefSeq NP_004985). The term “MMP-9” includes both the wild-type form of the nucleotide sequences or proteins as well as any mutants thereof. In some embodiments, “MMP-9” is wild-type MMP-9 receptor. In some embodiments, “MMP-9” is one or more mutant forms. The term “MMP-9” XYZ refers to a nucleotide sequence or protein of a mutant MMP-9 wherein the Y numbered amino acid of MMP-9 that normally has an X amino acid in the wildtype, instead has a Z amino acid in the mutant. In embodiments, an MMP-9 is the human MMP-9. In embodiments, the MMP-9 has the nucleotide sequence corresponding to reference number GI:74272286. In embodiments, the MMP-9 has the nucleotide sequence corresponding to RefSeq NM_004994.2. In embodiments, the MMP-9 has the protein sequence corresponding to reference number GI:74272287. In embodiments, the MMP-2 has the protein sequence corresponding to RefSeq NP_004985.2.

The term “polymeric micelle” refers to a micelle including the block copolymers (e.g. surfactant molecules) as described herein. The internal portion (e.g. core) of the polymeric micelle (also referred to herein as a “block copolymer micelle”) is hydrophobic while the exterior portion (e.g. shell) is hydrophilic. In embodiments, the polymeric micelle is a nanoparticle (referred to herein as “polymeric micelle nanoparticle”). In embodiments, the polymeric micelle is a spherical nanoparticle (referred to herein as a “spherical polymeric micelle nanoparticle”). A “responsive nanoparticle” and the like refer to a nanoparticle which contains a plurality of the block copolymers as described herein. A polymeric micelle is capable of undergoing a morphological transition from a discrete material (e.g., spherical-shaped micellar nanoparticle). In embodiments, the morphological transition is from a discrete material to a network-like assembly in response to environmental stimuli (e.g., enzymatic peptide cleavage). In embodiments, a polymeric micelle includes a hydrophobic core including hydrophobic polymerized monomers and a hydrophilic shell comprised of hydrophilic polymerized monomers.

A “nanoparticle,” as used herein, is a particle wherein the longest diameter is less than or equal to 1000 nanometers. In embodiments, a nanoparticle (e.g., polymeric micelle) is a particle wherein the longest diameter is less than or equal to 1000 nanometers including plurality of block copolymers. In embodiments, a nanoparticle has a shortest diameter greater than or equal to 1 nanometer (e.g., diameter from 1 to 1000 nanometers). In embodiments, the nanoparticle constructs provided herein may be an approximately spherical shape (referred to herein as a “spherical nanoparticle”).

The term “block copolymer” is used in accordance with its ordinary meaning and refers to a molecule including repeating subunits, also commonly referred to as monomers (e.g., polymerizable monomers). In embodiments, block copolymer including a peptide sequence (e.g., an inflammatory protease cleavable amino acid sequence, a cardiovascular inflammatory cleavable amino acid sequence, an MMP cleavable amino acid sequence), which may be referred to herein as a peptide-polymer amphiphile (PPA), a brush (e.g., polymers adhered to a surface) peptide-polymer amphiphile, or “amphiphilic block copolymer,” resulting from the composition containing both hydrophilic and hydrophobic portions. Such block copolymers may self-assemble into a polymeric micelle. In embodiments, the block copolymer includes two or more monomers. In embodiments, the block copolymer includes two or more monomers which are independently unique. In embodiments, the block copolymer includes two or more monomers in a periodic (e.g., repeating pattern) sequence.

The term “polymerizable monomer” is used in accordance with its meaning in the art of polymer chemistry and refers to a compound that may covalently bind chemically to other monomer molecules (such as other polymerizable monomers that are the same or different) to form a polymer thereby forming a polymerized monomer. An example of a polymerizable monomer is a ROMP polymerizable monomer, which is a polymerizable monomer capable of binding chemically to other ROMP polymerizable monomers through a ROMP chemical reaction (ring-opening metathesis polymerization) to form a polymer. It will be understood that a polymerizable monomer may be chemically modified in the polymerization reaction to differ from the free polymerizable monomer when forming the polymerized monomer moiety. In embodiments, the ROMP polymerizable monomer includes an olefin. In embodiments, the ROMP polymerizable monomer includes a cyclic olefin. In embodiments, the ROMP polymerizable monomer includes a cyclic olefin with ring strain (e.g., norbornene or cyclopentene or derivatives thereof). In embodiments, the ROMP polymerizable monomer is attached to a polypeptide. In embodiments, the ROMP polymerizable monomer is attached to a hydrophobic moiety. In embodiments, the ROMP polymerizable monomer is or includes a substituted or unsubstituted norbornenyl.

In embodiments, a polymerizable monomer is selected from:

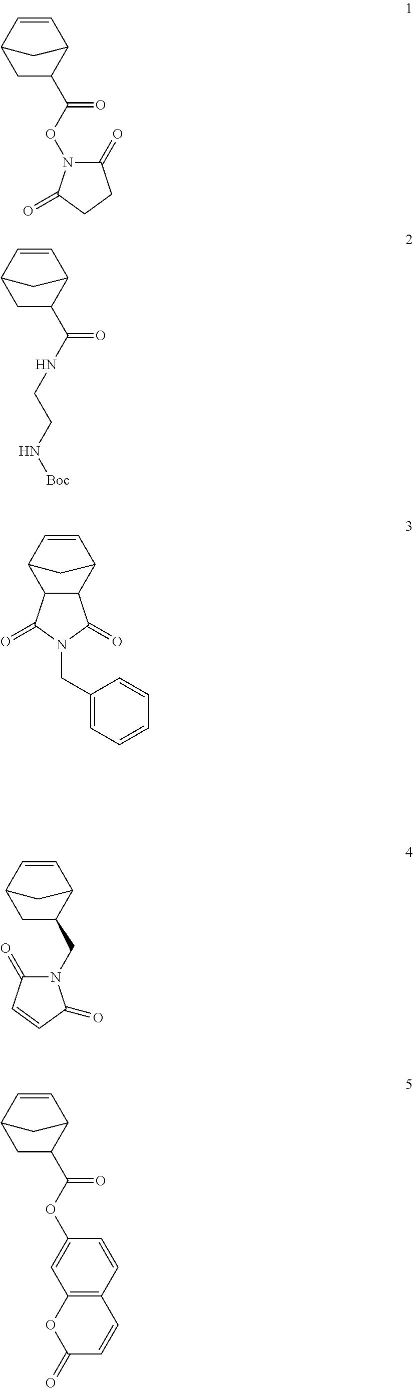

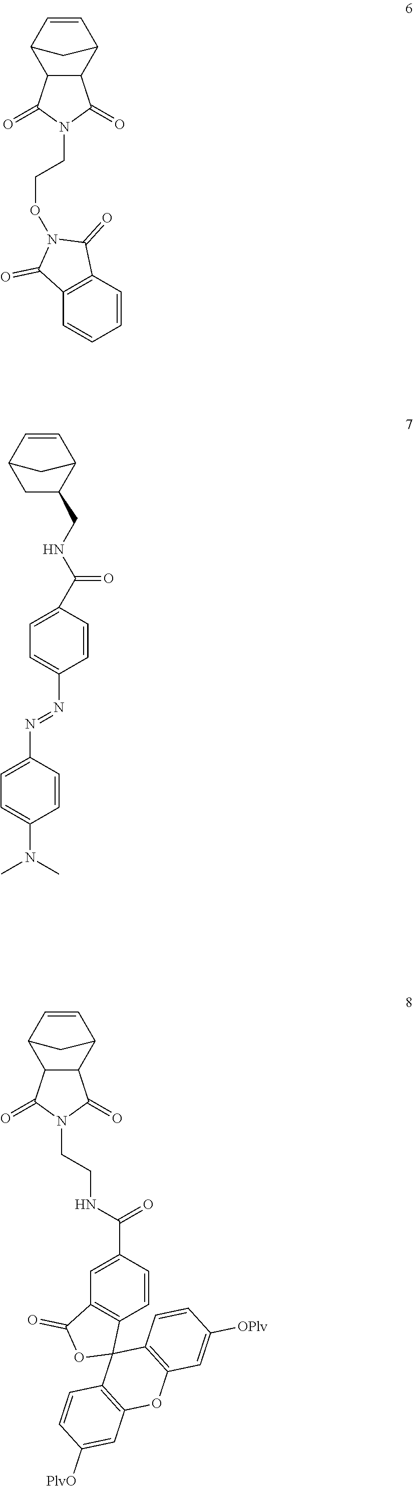

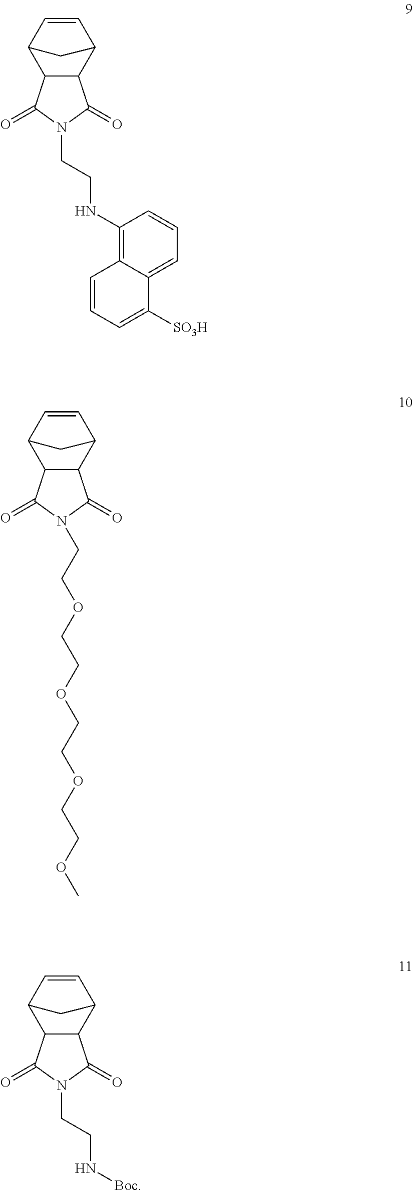 [SMILES: O=C1C2C3C=CC(C3)C2C(=O)N1CCNc1cccc2c(S(=O)(=O)O)cccc12 ; COCCOCCOCCOCCN1C(=O)C2C3C=CC(C3)C2C1=O ; CC(C)(C)OC(=O)NCCN1C(=O)C2C3C=CC(C3)C2C1=O]

The above polymerizable monomers form the polymerized monomers within the block copolymers disclosed herein.

The term “ring-opening metathesis polymerization” or “ROMP” is used in accordance with its meaning in polymer chemistry and refers to a chain-growth polymerization (e.g., olefin metathesis chain-growth polymerization). In embodiments, the reaction is driven by relief of ring strain in cyclic olefins (e.g., norbornene or cyclopentene). In embodiments, the ROMP uses a ruthenium catalyst. In embodiments, the ROMP uses a Grubbs' catalyst. In embodiments, the ROMP uses a Mo catalyst.

The terms “polymeric aggregate,” “micron scale aggregate”, “amphiphilic aggregate” and “aggregate assembly” are used herein synonymously in the context of a plurality of nanoparticles or copolymers disclosed herein which coalesce into network-like assemblies in response to an environmental stimuli (e.g., enzymatic peptide cleavage). For example, a polymeric micelle forms a disordered structure following cleavage of the MMP cleavable moiety. The disordered structure may then aggregate to form a network-like assembly (e.g. aggregation including hydrophobic interactions).

The term “targeting moiety” as used herein refers to a moiety that can be covalently or noncovalently attached to a compound (e.g., block copolymer) or biomolecule that serves as a recognition segment for a biological target (e.g. protein, tissue, or cell) and thereby helps in localization of the polymeric micelles provided herein to a target of interest. In embodiments, the targeting moiety is covalently attached. The targeting moiety may be an amino acid sequence, peptide or protein. The targeting moiety may be an antibody. In embodiments, the targeting moiety is present only within the second block of hydrophilic polymerized monomers of the block polymers provided herein and not within the first block of hydrophobic polymerized monomers. Targeting moieties include antibodies, peptide sequences, nucleic acids and small molecules; for example, peptides capable of recognizing receptors including integrins and small molecules capable of binding overexpressed receptors including folic acid receptor.

The term “drug moiety” used herein refers to a therapeutic agent that can be covalently or noncovalently attached to a compound (e.g., block copolymer) or biomolecule that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms (e.g., myocardial infarction) or the intended therapeutic effect, e.g., treatment or amelioration of an injury, disease, pathology or condition, or their symptoms (e.g., myocardial infarction) including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “surgical procedure” is used in accordance with its ordinary meaning and refers to an invasive therapy involving an incision performed in a patient. Non-limiting examples of surgical procedures include coronary ligation, coronary artery bypass grafting, transmyocardial laser revascularization, pacemaker placement, heart transplant, left ventricular assist device placement, aneurysm repair, mitrial valve surgery, cardiothoracic surgery, angioplasty, and stent placement.

### II. Compounds

In an aspect, provided herein is a block copolymer including a first block of hydrophobic polymerized monomers and a second block of hydrophilic polymerized monomers, wherein the first block of hydrophobic polymerized monomers includes a hydrophobic moiety covalently attached to each first block monomer backbone moiety within the first block of hydrophobic polymerized monomers, wherein each hydrophobic moiety is optionally different; and the second block of hydrophilic polymerized monomers includes a hydrophilic moiety covalently attached to each second block monomer backbone moiety within the second block of hydrophilic polymerized monomers, wherein each hydrophilic moiety is optionally different, and wherein at least one of the hydrophilic moieties includes an inflammatory protease cleavable amino acid sequence. In embodiments, the inflammatory protease cleavable amino acid sequence is a cardiovascular inflammatory protease cleavable amino acid sequence. In embodiments, the inflammatory protease cleavable amino acid sequence is a myocardial inflammatory protease cleavable amino acid sequence. In embodiments, the inflammatory protease cleavable amino acid sequence is an MMP-2 cleavable amino acid sequence. In embodiments, the inflammatory protease cleavable amino acid sequence is an MMP-9 cleavable amino acid sequence.

In an aspect is provided a block copolymer including a first block of hydrophobic polymerized monomers and a second block of hydrophilic polymerized monomers, wherein the first block of hydrophobic polymerized monomers include a hydrophobic moiety covalently attached to each first block monomer backbone moiety within the first block of hydrophobic polymerized monomers, wherein each hydrophobic moiety is optionally different; and the second block of hydrophilic polymerized monomers include a hydrophilic moiety covalently attached to each second block monomer backbone moiety within the second block of hydrophilic polymerized monomers, wherein each hydrophilic moiety is optionally different, and wherein at least one of the hydrophilic moieties includes an MMP-9 or MMP-2 cleavable amino acid sequence.

In embodiments, the hydrophobic moieties are the same. In embodiments, the hydrophobic moieties are different. In embodiments, each of the hydrophobic moieties is either: (a) a first hydrophobic moiety including a hydrophobic drug moiety, targeting moiety, or a detectable moiety or (b) a second hydrophobic moiety not including a hydrophobic drug moiety. In embodiments, each of the hydrophobic moieties is a first hydrophobic moiety including a hydrophobic drug moiety, targeting moiety, or a detectable moiety. In embodiments, each of the hydrophobic moieties is a second hydrophobic moiety not including a hydrophobic drug moiety. In embodiments, each of the hydrophobic moieties is a first hydrophobic moiety including a hydrophobic drug moiety. In embodiments, each of the hydrophobic moieties is a first hydrophobic moiety including a hydrophobic targeting moiety. In embodiments, each of the hydrophobic moieties is a first hydrophobic moiety including a hydrophobic detectable moiety. In embodiments, each of the hydrophobic moieties is a first hydrophobic moiety including a drug moiety. In embodiments, each of the hydrophobic moieties is a first hydrophobic moiety including a targeting moiety. In embodiments, each of the hydrophobic moieties is a first hydrophobic moiety including a detectable moiety.

In embodiments, each of the hydrophilic moieties is either: (a) a first hydrophilic moiety including an MMP-9 or MMP-2 cleavable amino acid sequence or (b) a second hydrophilic moiety not including an amino acid sequence. In embodiments, each of the hydrophilic moieties is a first hydrophilic moiety including an MMP-9 or MMP-2 cleavable amino acid sequence. In embodiments, each of the hydrophilic moieties is a first hydrophilic moiety including an MMP-9 cleavable amino acid sequence. In embodiments, each of the hydrophilic moieties is a first hydrophilic moiety including an MMP-2 cleavable amino acid sequence. In embodiments, each of the hydrophilic moieties is a second hydrophilic moiety not including an amino acid sequence.

In embodiments, the block copolymer has the formula:

R¹-L¹-[(A(-L²-R²))_{z1}—(B(-L³-R³))_{z2}-(A(-L²-R²))_{z3}]_{z4}—[(C(-L⁴-R⁴))_{z5}-(D(-L⁵-R⁵))_{z6}—(C(-L⁴-R⁴))_{z7})]_{z8}-L⁶-R⁶.

[(A(-L²-R²))_{z1}—(B(-L³-R³))_{z2}-(A(-L²-R²))_{z3}]_{z4} is the first block of hydrophobic polymerized monomers. [(C(-L⁴-R⁴))_{z5}-(D(-L⁵-R⁵))_{z6}—(C(-L⁴-R⁴))_{z7})]_{z8} is the second block of hydrophilic polymerized monomers. A and B are first block monomer backbone moieties. C and D are second block monomer backbone moieties. A “block monomer backbone moiety,” as used herein, refers to a chemical moiety that forms part of a monomeric unit of a polymer that, in combination with adjacent block monomer backbone moieties, forms a polymer backbone. The symbols z1, z3, z5 and z7 are independently integers from 0 to 100. The symbols z2, z4, z6 and z8 are independently integers from 1 to 100. L¹ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a hydrophobic drug moiety, a targeting moiety or a detectable moiety. L²-R² is a hydrophobic moiety, wherein L² is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a hydrophobic drug moiety, a targeting moiety or a detectable moiety. L³-R³ is a hydrophobic moiety, wherein L³ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L⁴-R⁴ is a hydrophilic moiety, wherein L⁴ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁴ is hydrogen, —OH, —SH, —NH₂, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a hydrophilic drug moiety, a targeting moiety or a detectable moiety. L⁵-R⁵ is a hydrophilic moiety, wherein L⁵ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and R⁵ is an amino acid sequence including the inflammatory protease (e.g., cardiovascular inflammatory protease, myocardial inflammatory protease, MMP-9, or MMP-2) cleavable amino acid sequence. L⁶ is a bond, —O—, —S—, —NH—, —C(O)—, —C(O)O—, —C(O)NH—, -L^{6A}-L^{6B}-L^{6C}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁶ is hydrogen, —OH, —SH, —NH₂, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a hydrophilic drug moiety, a targeting moiety or a detectable moiety. L^{6A}, L^{6B} and L^{6C} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{6A}, L^{6B} and L^{6C} are independently a bond substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, R¹ is independently hydrogen, oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, R⁷-substituted or unsubstituted alkyl (e.g., C₁-C₈ alkyl, C₁-C₆ alkyl, or C₁-C₄ alkyl), R⁷-substituted or unsubstituted heteroalkyl (e.g., 2 to 8 membered heteroalkyl, 2 to 6 membered heteroalkyl, or 2 to 4 membered heteroalkyl), R⁷-substituted or unsubstituted cycloalkyl (e.g., C₃-C₈ cycloalkyl, C₃-C₆ cycloalkyl, or C₅-C₆ cycloalkyl), R⁷-substituted or unsubstituted heterocycloalkyl (e.g., 3 to 8 membered heterocycloalkyl, 3 to 6 membered heterocycloalkyl, or 5 to 6 membered heterocycloalkyl), R⁷-substituted or unsubstituted aryl (e.g., C₆-C₁₀ aryl, C₁₀ aryl, or phenyl), or R⁷-substituted or unsubstituted heteroaryl (e.g., 5 to 10 membered heteroaryl, 5 to 9 membered heteroaryl, or 5 to 6 membered heteroaryl), a hydrophobic drug moiety, a targeting moiety or a detectable moiety.

In embodiments, R¹ is R⁷-substituted or unsubstituted alkyl (e.g., C₁-C₈ alkyl, C₁-C₆ alkyl, or C₁-C₄ alkyl). In embodiments, R¹ is R⁷-substituted alkyl (e.g., C₁-C₈ alkyl, C₁-C₆ alkyl, or C₁-C₄ alkyl). In embodiments, R¹ is an unsubstituted alkyl (e.g., C₁-C₈ alkyl, C₁-C₆ alkyl, or C₁-C₄ alkyl).

In embodiments, R¹ is R⁷-substituted or unsubstituted heteroalkyl (e.g., 2 to 8 membered heteroalkyl, 2 to 6 membered heteroalkyl, or 2 to 4 membered heteroalkyl). In embodiments, R¹ is R⁷-substituted heteroalkyl (e.g., 2 to 8 membered heteroalkyl, 2 to 6 membered heteroalkyl, or 2 to 4 membered heteroalkyl). In embodiments, R¹ is an unsubstituted heteroalkyl (e.g., 2 to 8 membered heteroalkyl, 2 to 6 membered heteroalkyl, or 2 to 4 membered heteroalkyl).

In embodiments, R¹ is R⁷-substituted or unsubstituted cycloalkyl (e.g., C₃-C₈ cycloalkyl, C₃-C₆ cycloalkyl, or C₅-C₆ cycloalkyl). In embodiments, R¹ is R⁷-substituted cycloalkyl (e.g., C₃-C₈ cycloalkyl, C₃-C₆ cycloalkyl, or C₅-C₆ cycloalkyl). In embodiments, R¹ is an unsubstituted cycloalkyl (e.g., C₃-C₈ cycloalkyl, C₃-C₆ cycloalkyl, or C₅-C₆ cycloalkyl).

In embodiments, R¹ is R⁷-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹ is R⁷-substituted heterocycloalkyl (e.g., 3 to 8 membered heterocycloalkyl, 3 to 6 membered heterocycloalkyl, or 5 to 6 membered heterocycloalkyl). In embodiments, R¹ is an unsubstituted heterocycloalkyl (e.g., 3 to 8 membered heterocycloalkyl, 3 to 6 membered heterocycloalkyl, or 5 to 6 membered heterocycloalkyl).

In embodiments, R¹ is R⁷-substituted or unsubstituted aryl (e.g., C₆-C₁₀ aryl, C₁₀ aryl, or phenyl). In embodiments, R¹ is R⁷-substituted aryl (e.g., C₆-C₁₀ aryl, C₁₀ aryl, or phenyl). In embodiments, R¹ is an unsubstituted aryl (e.g., C₆-C₁₀ aryl, C₁₀ aryl, or phenyl). In embodiments, R¹ is an unsubstituted phenyl.

In embodiments, R¹ is R⁷-substituted or unsubstituted heteroaryl (e.g., 5 to 10 membered heteroaryl, 5 to 9 membered heteroaryl, or 5 to 6 membered heteroaryl). In embodiments, R¹ is R⁷-substituted heteroaryl (e.g., 5 to 10 membered heteroaryl, 5 to 9 membered heteroaryl, or 5 to 6 membered heteroaryl). In embodiments, R¹ is an unsubstituted heteroaryl (e.g., 5 to 10 membered heteroaryl, 5 to 9 membered heteroaryl, or 5 to 6 membered heteroaryl).

In embodiments, R¹ is a hydrophobic drug moiety, a targeting moiety or a detectable moiety. In embodiments, R¹ is a hydrophobic drug moiety. In embodiments, R¹ is a drug moiety. In embodiments, R¹ is a targeting moiety. In embodiments, R¹ is a detectable moiety. In embodiments, R¹ is hydrogen.

R⁷ is independently oxo,

halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, R⁸-substituted or unsubstituted alkyl (e.g., C₁-C₈ alkyl, C₁-C₆ alkyl, or C₁-C₄ alkyl), R⁸-substituted or unsubstituted heteroalkyl (e.g., 2 to 8 membered heteroalkyl, 2 to 6 membered heteroalkyl, or 2 to 4 membered heteroalkyl), R⁸-substituted or unsubstituted cycloalkyl (e.g., C₃-C₈ cycloalkyl, C₃-C₆ cycloalkyl, or C₅-C₆ cycloalkyl), R⁸-substituted or unsubstituted heterocycloalkyl (e.g., 3 to 8 membered heterocycloalkyl, 3 to 6 membered heterocycloalkyl, or 5 to 6 membered heterocycloalkyl), R⁸-substituted or unsubstituted aryl (e.g., C₆-C₁₀ aryl, C₁₀ aryl, or phenyl), or R⁸-substituted or unsubstituted heteroaryl (e.g., 5 to 10 membered heteroaryl, 5 to 9 membered heteroaryl, or 5 to 6 membered heteroaryl).

In embodiments, L¹ is a bond, —O—, —S—, —NH—, —C(O)—, —C(O)O—, —C(O)NH—, R⁹-substituted or unsubstituted alkylene (e.g., C₁-C₈ alkylene, C₁-C₆ alkylene, or C₁-C₄ alkylene), R⁹-substituted or unsubstituted heteroalkylene (e.g., 2 to 8 membered heteroalkylene, 2 to 6 membered heteroalkylene, or 2 to 4 membered heteroalkylene), R⁹-substituted or unsubstituted cycloalkylene (e.g., C₃-C₈ cycloalkylene, C₃-C₆ cycloalkylene, or C₅-C₆ cycloalkylene), R⁹-substituted or unsubstituted heterocycloalkylene (e.g., 3 to 8 membered heterocycloalkylene, 3 to 6 membered heterocycloalkylene, or 5 to 6 membered heterocycloalkylene), R⁹-substituted or unsubstituted arylene (e.g., C₆-C₁₀ arylene, C₁₀ arylene, or phenylene), or R⁹-substituted or unsubstituted heteroarylene (e.g., 5 to 10 membered heteroarylene, 5 to 9 membered heteroarylene, or 5 to 6 membered heteroarylene). In embodiments, L¹ is a bond, —O—, —S—, —NH—, —C(O)—, —C(O)O—, —C(O)NH—, R⁹-substituted or unsubstituted alkenylene (e.g., C₂-C₈ alkenylene, C₂-C₆ alkenylene, or C₂-C₄ alkenylene). In embodiments, L¹ is a substituted or unsubstituted ethylenyl.

In embodiments, L¹ is R⁹-substituted or unsubstituted alkylene (e.g., C₁-C₈ alkylene, C₁-C₆ alkylene, or C₁-C₄ alkylene). In embodiments, L¹ is R⁹-substituted alkylene (e.g., C₁-C₈ alkylene, C₁-C₆ alkylene, or C₁-C₄ alkylene). In embodiments, L¹ is an unsubstituted alkylene (e.g., C₁-C₈ alkylene, C₁-C₆ alkylene, or C₁-C₄ alkylene).

In embodiments, L¹ is R⁹-substituted or unsubstituted heteroalkylene (e.g., 2 to 8 membered heteroalkylene, 2 to 6 membered heteroalkylene, or 2 to 4 membered heteroalkylene). In embodiments, L¹ is R⁹-substituted heteroalkylene (e.g., 2 to 8 membered heteroalkylene, 2 to 6 membered heteroalkylene, or 2 to 4 membered heteroalkylene). In embodiments, L¹ is an unsubstituted heteroalkylene (e.g., 2 to 8 membered heteroalkylene, 2 to 6 membered heteroalkylene, or 2 to 4 membered heteroalkylene).

In embodiments, L¹ is R⁹-substituted or unsubstituted cycloalkylene (e.g., C₃-C₈ cycloalkylene, C₃-C₆ cycloalkylene, or C₅-C₆ cycloalkylene). In embodiments, L¹ is R⁹-substituted cycloalkylene (e.g., C₃-C₈ cycloalkylene, C₃-C₆ cycloalkylene, or C₅-C₆ cycloalkylene). In embodiments, L¹ is an unsubstituted cycloalkylene (e.g., C₃-C₈ cycloalkylene, C₃-C₆ cycloalkylene, or C₅-C₆ cycloalkylene).

In embodiments, L¹ is R⁹-substituted or unsubstituted heterocycloalkylene (e.g., 3 to 8 membered heterocycloalkylene, 3 to 6 membered heterocycloalkylene, or 5 to 6 membered heterocycloalkylene). In embodiments, L¹ is R⁹-substituted heterocycloalkylene (e.g., 3 to 8 membered heterocycloalkylene, 3 to 6 membered heterocycloalkylene, or 5 to 6 membered heterocycloalkylene). In embodiments, L¹ is an unsubstituted heterocycloalkylene (e.g., 3 to 8 membered heterocycloalkylene, 3 to 6 membered heterocycloalkylene, or 5 to 6 membered heterocycloalkylene).

In embodiments, L¹ is R⁹-substituted or unsubstituted arylene (e.g., C₆-C10 arylene, C₁₀ arylene, or phenylene). In embodiments, L¹ is R⁹-substituted arylene (e.g., C₆-C10 arylene, C₁₀ arylene, or phenylene). In embodiments, L¹ is an unsubstituted arylene (e.g., C₆-C10 arylene, C10 arylene, or phenylene).

In embodiments, L¹ is R⁹-substituted or unsubstituted heteroarylene (e.g., 5 to 10 membered heteroarylene, 5 to 9 membered heteroarylene, or 5 to 6 membered heteroarylene). In embodiments, L¹ is R⁹-substituted heteroarylene (e.g., 5 to 10 membered heteroarylene, 5 to 9 membered heteroarylene, or 5 to 6 membered heteroarylene). In embodiments, L¹ is an unsubstituted heteroarylene (e.g., 5 to 10 membered heteroarylene, 5 to 9 membered heteroarylene, or 5 to 6 membered heteroarylene). In embodiments, L¹ is a bond.

R⁹ is independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, R¹⁰-substituted or unsubstituted alkyl (e.g., C₁-C₈ alkyl, C₁-C₆ alkyl, or C₁-C₄ alkyl), R¹⁰-substituted or unsubstituted heteroalkyl (e.g., 2 to 8 membered heteroalkyl, 2 to 6 membered heteroalkyl, or 2 to 4 membered heteroalkyl), R¹⁰-substituted or unsubstituted cycloalkyl (e.g., C₃-C₈ cycloalkyl, C₃-C₆ cycloalkyl, or C₅-C₆ cycloalkyl), R¹⁰-substituted or unsubstituted heterocycloalkyl (e.g., 3 to 8 membered heterocycloalkyl, 3 to 6 membered heterocycloalkyl, or 5 to 6 membered heterocycloalkyl), R¹⁰-substituted or unsubstituted aryl (e.g., C₆-C10 aryl, C₁₀ aryl, or phenyl), or R¹⁰-substituted or unsubstituted heteroaryl (e.g., 5 to 10 membered heteroaryl, 5 to 9 membered heteroaryl, or 5 to 6 membered heteroaryl).

In embodiments, A is a first block monomer backbone moiety. Thus in embodiments, (A(-L²-R²))_{z1} has the formula (-L^{1A}-A¹(-L²-R²)-L^{2A}-)_{z1}. L^{1A} and L^{2A} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. A¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, A¹ is R²⁵-substituted or unsubstituted alkylene (e.g., C₁-C₈ alkylene, C₁-C₆ alkylene, or C₁-C₄ alkylene), R²⁵-substituted or unsubstituted heteroalkylene (e.g., 2 to 8 membered heteroalkylene, 2 to 6 membered heteroalkylene, or 2 to 4 membered heteroalkylene), R²⁵-substituted or unsubstituted cycloalkylene (e.g., C₃-C₈ cycloalkylene, C₃-C₆ cycloalkylene, or C₅-C₆ cycloalkylene), R²⁵-substituted or unsubstituted heterocycloalkylene (e.g., 3 to 8 membered heterocycloalkylene, 3 to 6 membered heterocycloalkylene, or 5 to 6 membered heterocycloalkylene), R²⁵-substituted or unsubstituted arylene (e.g., C₆-C₁₀ arylene, C₁₀ arylene, or phenylene), or R²⁵-substituted or unsubstituted heteroarylene (e.g., 5 to 10 membered heteroarylene, 5 to 9 membered heteroarylene, or 5 to 6 membered heteroarylene).

In embodiments, A¹ is R²⁵-substituted or unsubstituted alkylene (e.g., C₁-C₈ alkylene, C₁-C₆ alkylene, or C₁-C₄ alkylene). In embodiments, A¹ is R²⁵-substituted alkylene (e.g., C₁-C₈ alkylene, C₁-C₆ alkylene, or C₁-C₄ alkylene). In embodiments, A¹ is an unsubstituted alkylene (e.g., C₁-C₈ alkylene, C₁-C₆ alkylene, or C₁-C₄ alkylene).

In embodiments, A¹ is R²⁵-substituted or unsubstituted heteroalkylene (e.g., 2 to 8 membered heteroalkylene, 2 to 6 membered heteroalkylene, or 2 to 4 membered heteroalkylene). In embodiments, A¹ is R²⁵-substituted heteroalkylene (e.g., 2 to 8 membered heteroalkylene, 2 to 6 membered heteroalkylene, or 2 to 4 membered heteroalkylene). In embodiments, A¹ is an unsubstituted heteroalkylene (e.g., 2 to 8 membered heteroalkylene, 2 to 6 membered heteroalkylene, or 2 to 4 membered heteroalkylene).

In embodiments, A¹ is R²⁵-substituted or unsubstituted cycloalkylene (e.g., C₃-C₈ cycloalkylene, C₃-C₆ cycloalkylene, or C₅-C₆ cycloalkylene). In embodiments, A¹ is R²⁵-substituted cycloalkylene (e.g., C₃-C₈ cycloalkylene, C₃-C₆ cycloalkylene, or C₅-C₆ cycloalkylene). In embodiments, A¹ is an unsubstituted cycloalkylene (e.g., C₃-C₈ cycloalkylene, C₃-C₆ cycloalkylene, or C₅-C₆ cycloalkylene).

In embodiments, A¹ is R²⁵-substituted or unsubstituted heterocycloalkylene (e.g., 3 to 8 membered heterocycloalkylene, 3 to 6 membered heterocycloalkylene, or 5 to 6 membered heterocycloalkylene). In embodiments, A¹ is R²⁵-substituted heterocycloalkylene (e.g., 3 to 8 membered heterocycloalkylene, 3 to 6 membered heterocycloalkylene, or 5 to 6 membered heterocycloalkylene). In embodiments, A¹ is an unsubstituted heterocycloalkylene (e.g., 3 to 8 membered heterocycloalkylene, 3 to 6 membered heterocycloalkylene, or 5 to 6 membered heterocycloalkylene).

In embodiments, A¹ is R²⁵-substituted or unsubstituted arylene (e.g., C₆-C₁₀ arylene, C₁₀ arylene, or phenylene). In embodiments, A¹ is R²⁵-substituted arylene (e.g., C₆-C₁₀ arylene, C₁₀ arylene, or phenylene). In embodiments, A¹ is an unsubstituted arylene (e.g., C₆-C₁₀ arylene, C₁₀ arylene, or phenylene).

In embodiments, A¹ is R²⁵-substituted or unsubstituted heteroarylene (e.g., 5 to 10 membered heteroarylene, 5 to 9 membered heteroarylene, or 5 to 6 membered heteroarylene). In embodiments, A¹ is R²⁵-substituted heteroarylene (e.g., 5 to 10 membered heteroarylene, 5 to 9 membered heteroarylene, or 5 to 6 membered heteroarylene). In embodiments, A¹ is an unsubstituted heteroarylene (e.g., 5 to 10 membered heteroarylene, 5 to 9 membered heteroarylene, or 5 to 6 membered heteroarylene).

R²⁵ is independently oxo,

halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, R²⁶-substituted or unsubstituted alkyl (e.g., C₁-C₈ alkyl, C₁-C₆ alkyl, or C₁-C₄ alkyl), R²⁶-substituted or unsubstituted heteroalkyl (e.g., 2 to 8 membered heteroalkyl, 2 to 6 membered heteroalkyl, or 2 to 4 membered heteroalkyl), R²⁶-substituted or unsubstituted cycloalkyl (e.g., C₃-C₈ cycloalkyl, C₃-C₆ cycloalkyl, or C₅-C₆ cycloalkyl), R²⁶-substituted or unsubstituted heterocycloalkyl (e.g., 3 to 8 membered heterocycloalkyl, 3 to 6 membered heterocycloalkyl, or 5 to 6 membered heterocycloalkyl), R²⁶-substituted or unsubstituted aryl (e.g., C₆-C₁₀ aryl, C₁₀ aryl, or phenyl), or R²⁶-substituted or unsubstituted heteroaryl (e.g., 5 to 10 membered heteroaryl, 5 to 9 membered heteroaryl, or 5 to 6 membered heteroaryl).

In embodiments, R²⁵ is oxo, —OH, —NH₂, —COOH, or —CONH₂. In embodiments, R²⁵ is oxo.

In embodiments, L^{1A} is a bond, —O—, —S—, —NH—, —C(O)—, —C(O)O—, —C(O)NH—, R^{22A}-substituted or unsubstituted alkylene (e.g., C₁-C₈ alkylene, C₁-C₆ alkylene, or C₁-C₄ alkylene), R^{22A}-substituted or unsubstituted heteroalkylene (e.g., 2 to 8 membered heteroalkylene, 2 to 6 membered heteroalkylene, or 2 to 4 membered heteroalkylene), R^{22A}-substituted or unsubstituted cycloalkylene (e.g., C₃-C₈ cycloalkylene, C₃-C₆ cycloalkylene, or C₅-C₆ cycloalkylene), R^{22A}-substituted or unsubstituted heterocycloalkylene (e.g., 3 to 8 membered heterocycloalkylene, 3 to 6 membered heterocycloalkylene, or 5 to 6 membered heterocycloalkylene), R^{22A}-substituted or unsubstituted arylene (e.g., C₆-C₁₀ arylene, C₁₀ arylene, or phenylene), or R^{22A}-substituted or unsubstituted heteroarylene (e.g., 5 to 10 membered heteroarylene, 5 to 9 membered heteroarylene, or 5 to 6 membered heteroarylene). In embodiments, L^{1A} is a bond, —O—, —S—, —NH—, —C(O)—, —C(O)O—, —C(O)NH—, R^{22A}-substituted or unsubstituted alkenylene (e.g., C₂-C₈ alkenylene, C₂-C₆ alkenylene, or C₂-C₄ alkenylene). In embodiments, L^{1A} is a substituted or unsubstituted ethylenyl.

In embodiments, L^{2A} is a bond, —O—, —S—, —NH—, —C(O)—, —C(O)O—, —C(O)NH—, R^{23A}-substituted or unsubstituted alkylene (e.g., C₁-C₈ alkylene, C₁-C₆ alkylene, or C₁-C₄ alkylene), R^{23A}-substituted or unsubstituted heteroalkylene (e.g., 2 to 8 membered heteroalkylene, 2 to 6 membered heteroalkylene, or 2 to 4 membered heteroalkylene), R^{23A}-substituted or unsubstituted cycloalkylene (e.g., C₃-C₈ cycloalkylene, C₃-C₆ cycloalkylene, or C₅-C₆ cycloalkylene), R^{23A}-substituted or unsubstituted heterocycloalkylene (e.g., 3 to 8 membered heterocycloalkylene, 3 to 6 membered heterocycloalkylene, or 5 to 6 membered heterocycloalkylene), R^{23A}-substituted or unsubstituted arylene (e.g., C₆-C₁₀ arylene, C₁₀ arylene, or phenylene), or R^{23A}-substituted or unsubstituted heteroarylene (e.g., 5 to 10 membered heteroarylene, 5 to 9 membered heteroarylene, or 5 to 6 membered heteroarylene). In embodiments, L^{2A} is a bond, —O—, —S—, —NH—, —C(O)—, —C(O)O—, —C(O)NH—, R^{23A}-substituted or unsubstituted alkenylene (e.g., C₂-C₈ alkenylene, C₂-C₆ alkenylene, or C₂-C₄ alkenylene). In embodiments, L^{2A} is a substituted or unsubstituted ethylenyl.

In embodiments, (A(-L²-R²))_{z1} has the formula:

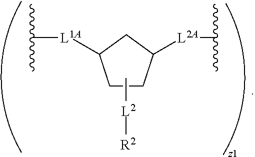

In embodiments, L^{1A} and L^{2A} are independently a bond, —O—, —S—, —NH—, —C(O)—, —C(O)O—, —C(O)NH—, substituted or unsubstituted alkylene, substituted or unsubstituted heteroalkylene.

In embodiments, (A(-L²-R²))_{z3} has the formula (-L^{1A}-A¹(-L²-R²)-L^{2A}-)_{z3}. In embodiments, (A(-L²-R²))_{z3} has the formula:

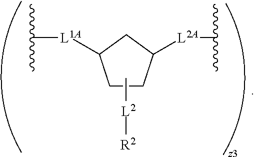

In embodiments, L^{1A} and L^{2A} are independently a bond, —O—, —S—, —NH—, —C(O)—, —C(O)O—, —C(O)NH—, substituted or unsubstituted alkylene, substituted or unsubstituted heteroalkylene.

In embodiments, L²-R² is a hydrophobic moiety. In embodiments, L² is a hydrophobic moiety. In embodiments, R² is a hydrophobic moiety.

In embodiments, L² is a bond, —O—, —S—, —NH—, —C(O)—, —C(O)O—, —C(O)NH—, R¹¹-substituted or unsubstituted alkylene (e.g., C₁-C₈ alkylene, C₁-C₆ alkylene, or C₁-C₄ alkylene), R¹¹-substituted or unsubstituted heteroalkylene (e.g., 2 to 8 membered heteroalkylene, 2 to 6 membered heteroalkylene, or 2 to 4 membered heteroalkylene), R¹¹-substituted or unsubstituted cycloalkylene (e.g., C₃-C₈ cycloalkylene, C₃-C₆ cycloalkylene, or C₅-C₆ cycloalkylene), R¹¹-substituted or unsubstituted heterocycloalkylene (e.g., 3 to 8 membered heterocycloalkylene, 3 to 6 membered heterocycloalkylene, or 5 to 6 membered heterocycloalkylene), R¹¹-substituted or unsubstituted arylene (e.g., C₆-C₁₀ arylene, C₁₀ arylene, or phenylene), or R¹¹-substituted or unsubstituted heteroarylene (e.g., 5 to 10 membered heteroarylene, 5 to 9 membered heteroarylene, or 5 to 6 membered heteroarylene). In embodiments, L² is a bond.

In embodiments, R² is hydrogen, R¹²-substituted or unsubstituted alkyl (e.g., C₁-C₈ alkyl, C₁-C₆ alkyl, or C₁-C₄ alkyl), R¹²-substituted or unsubstituted heteroalkyl (e.g., 2 to 8 membered heteroalkyl, 2 to 6 membered heteroalkyl, or 2 to 4 membered heteroalkyl), R¹²-substituted or unsubstituted cycloalkyl (e.g., C₃-C₈ cycloalkyl, C₃-C₆ cycloalkyl, or C₅-C₆ cycloalkyl), R¹²-substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), R¹²-substituted or unsubstituted heteroaryl (e.g., 5 to 10 membered heteroaryl, 5 to 9 membered heteroaryl, or 5 to 6 membered heteroaryl) a hydrophobic drug moiety, a targeting moiety or a detectable moiety. In embodiments, R² is hydrogen.

In embodiments, B is a first block monomer backbone moiety. Thus in embodiments, (B(-L³-R³))_{z2} has the formula (-L^{1B}-B¹(-L³-R³)-L^{2B}-)_{z2}. L^{1B} and L^{2B} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. B¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, B¹ is R²⁷-substituted or unsubstituted alkylene (e.g., C₁-C₈ alkylene, C₁-C₆ alkylene, or C₁-C₄ alkylene), R²⁷-substituted or unsubstituted heteroalkylene (e.g., 2 to 8 membered heteroalkylene, 2 to 6 membered heteroalkylene, or 2 to 4 membered heteroalkylene), R²⁷-substituted or unsubstituted cycloalkylene (e.g., C₃-C₈ cycloalkylene, C₃-C₆ cycloalkylene, or C₅-C₆ cycloalkylene), R²⁷-substituted or unsubstituted heterocycloalkylene (e.g., 3 to 8 membered heterocycloalkylene, 3 to 6 membered heterocycloalkylene, or 5 to 6 membered heterocycloalkylene), R²⁷-substituted or unsubstituted arylene (e.g., C₆-C₁₀ arylene, C₁₀ arylene, or phenylene), or R²⁷-substituted or unsubstituted heteroarylene (e.g., 5 to 10 membered heteroarylene, 5 to 9 membered heteroarylene, or 5 to 6 membered heteroarylene).

In embodiments, B¹ is R²⁷-substituted or unsubstituted alkylene (e.g., C₁-C₈ alkylene, C₁-C₆ alkylene, or C₁-C₄ alkylene). In embodiments, B¹ is R²⁷-substituted alkylene (e.g., C₁-C₈ alkylene, C₁-C₆ alkylene, or C₁-C₄ alkylene). In embodiments, B¹ is an unsubstituted alkylene (e.g., C₁-C₈ alkylene, C₁-C₆ alkylene, or C₁-C₄ alkylene).

In embodiments, B¹ is R²⁷-substituted or unsubstituted heteroalkylene (e.g., 2 to 8 membered heteroalkylene, 2 to 6 membered heteroalkylene, or 2 to 4 membered heteroalkylene). In embodiments, B¹ is R²⁷-substituted heteroalkylene (e.g., 2 to 8 membered heteroalkylene, 2 to 6 membered heteroalkylene, or 2 to 4 membered heteroalkylene). In embodiments, B¹ is an unsubstituted heteroalkylene (e.g., 2 to 8 membered heteroalkylene, 2 to 6 membered heteroalkylene, or 2 to 4 membered heteroalkylene).

In embodiments, B¹ is R²⁷-substituted or unsubstituted cycloalkylene (e.g., C₃-C₈ cycloalkylene, C₃-C₆ cycloalkylene, or C₅-C₆ cycloalkylene). In embodiments, B¹ is R²⁷-substituted cycloalkylene (e.g., C₃-C₈ cycloalkylene, C₃-C₆ cycloalkylene, or C₅-C₆ cycloalkylene). In embodiments, B¹ is an unsubstituted cycloalkylene (e.g., C₃-C₈ cycloalkylene, C₃-C₆ cycloalkylene, or C₅-C₆ cycloalkylene).

In embodiments, B¹ is R²⁷-substituted or unsubstituted heterocycloalkylene (e.g., 3 to 8 membered heterocycloalkylene, 3 to 6 membered heterocycloalkylene, or 5 to 6 membered heterocycloalkylene). In embodiments, B¹ is R²⁷-substituted heterocycloalkylene (e.g., 3 to 8 membered heterocycloalkylene, 3 to 6 membered heterocycloalkylene, or 5 to 6 membered heterocycloalkylene). In embodiments, B¹ is an unsubstituted heterocycloalkylene (e.g., 3 to 8 membered heterocycloalkylene, 3 to 6 membered heterocycloalkylene, or 5 to 6 membered heterocycloalkylene).

In embodiments, B¹ is R²⁷-substituted or unsubstituted arylene (e.g., C₆-C₁₀ arylene, C₁₀ arylene, or phenylene). In embodiments, B¹ is R²⁷-substituted arylene (e.g., C₆-C₁₀ arylene, C₁₀ arylene, or phenylene). In embodiments, B¹ is an unsubstituted arylene (e.g., C₆-C₁₀ arylene, C₁₀ arylene, or phenylene).

In embodiments, B¹ is R²⁷-substituted or unsubstituted heteroarylene (e.g., 5 to 10 membered heteroarylene, 5 to 9 membered heteroarylene, or 5 to 6 membered heteroarylene). In embodiments, B¹ is R²⁷-substituted heteroarylene (e.g., 5 to 10 membered heteroarylene, 5 to 9 membered heteroarylene, or 5 to 6 membered heteroarylene). In embodiments, B¹ is an unsubstituted heteroarylene (e.g., 5 to 10 membered heteroarylene, 5 to 9 membered heteroarylene, or 5 to 6 membered heteroarylene).

R²⁷ is independently oxo,

halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, R²⁸-substituted or unsubstituted alkyl (e.g., C₁-C₈ alkyl, C₁-C₆ alkyl, or C₁-C₄ alkyl), R²⁸-substituted or unsubstituted heteroalkyl (e.g., 2 to 8 membered heteroalkyl, 2 to 6 membered heteroalkyl, or 2 to 4 membered heteroalkyl), R²⁸-substituted or unsubstituted cycloalkyl (e.g., C₃-C₈ cycloalkyl, C₃-C₆ cycloalkyl, or C₅-C₆ cycloalkyl), R²⁸-substituted or unsubstituted heterocycloalkyl (e.g., 3 to 8 membered heterocycloalkyl, 3 to 6 membered heterocycloalkyl, or 5 to 6 membered heterocycloalkyl), R²⁸-substituted or unsubstituted aryl (e.g., C₆-C₁₀ aryl, C₁₀ aryl, or phenyl), or R²⁸-substituted or unsubstituted heteroaryl (e.g., 5 to 10 membered heteroaryl, 5 to 9 membered heteroaryl, or 5 to 6 membered heteroaryl).

In embodiments, R²⁷ is oxo, —OH, —NH₂, —COOH, or —CONH₂. In embodiments, R²⁷ is oxo.

In embodiments, L^{1B} is a bond, —O—, —S—, —NH—, —C(O)—, —C(O)O—, —C(O)NH—, R^{22B}-substituted or unsubstituted alkylene (e.g., C₁-C₈ alkylene, C₁-C₆ alkylene, or C₁-C₄ alkylene), R^{22B}-substituted or unsubstituted heteroalkylene (e.g., 2 to 8 membered heteroalkylene, 2 to 6 membered heteroalkylene, or 2 to 4 membered heteroalkylene), R^{22B}-substituted or unsubstituted cycloalkylene (e.g., C₃-C₈ cycloalkylene, C₃-C₆ cycloalkylene, or C₅-C₆ cycloalkylene), R^{22B}-substituted or unsubstituted heterocycloalkylene (e.g., 3 to 8 membered heterocycloalkylene, 3 to 6 membered heterocycloalkylene, or 5 to 6 membered heterocycloalkylene), R^{22B}-substituted or unsubstituted arylene (e.g., C₆-C₁₀ arylene, C₁₀ arylene, or phenylene), or R^{22B}-substituted or unsubstituted heteroarylene (e.g., 5 to 10 membered heteroarylene, 5 to 9 membered heteroarylene, or 5 to 6 membered heteroarylene). In embodiments, L^{1B} is a bond, —O—, —S—, —NH—, —C(O)—, —C(O)O—, —C(O)NH—, R^{22B}-substituted or unsubstituted alkenylene (e.g., C₂-C₈ alkenylene, C₂-C₆ alkenylene, or C₂-C₄ alkenylene). In embodiments, L^{1B} is a substituted or unsubstituted ethylenyl.

In embodiments, L^{2B} is a bond, —O—, —S—, —NH—, —C(O)—, —C(O)O—, —C(O)NH—, R^{23B}-substituted or unsubstituted alkylene (e.g., C₁-C₈ alkylene, C₁-C₆ alkylene, or C₁-C₄ alkylene), R^{23B}-substituted or unsubstituted heteroalkylene (e.g., 2 to 8 membered heteroalkylene, 2 to 6 membered heteroalkylene, or 2 to 4 membered heteroalkylene), R^{23B}-substituted or unsubstituted cycloalkylene (e.g., C₃-C₈ cycloalkylene, C₃-C₆ cycloalkylene, or C₅-C₆ cycloalkylene), R^{23B}-substituted or unsubstituted heterocycloalkylene (e.g., 3 to 8 membered heterocycloalkylene, 3 to 6 membered heterocycloalkylene, or 5 to 6 membered heterocycloalkylene), R^{23B}-substituted or unsubstituted arylene (e.g., C₆-C₁₀ arylene, C₁₀ arylene, or phenylene), or R^{23B}-substituted or unsubstituted heteroarylene (e.g., 5 to 10 membered heteroarylene, 5 to 9 membered heteroarylene, or 5 to 6 membered heteroarylene). In embodiments, L^{2B} is a bond, —O—, —S—, —NH—, —C(O)—, —C(O)O—, —C(O)NH—, R^{23B}-substituted or unsubstituted alkenylene (e.g., C₂-C₈ alkenylene, C₂-C₆ alkenylene, or C₂-C₄ alkenylene). In embodiments, L^{2B} is a substituted or unsubstituted ethylenyl.

In embodiments, (B(-L³-R³))_{z2} has the formula:

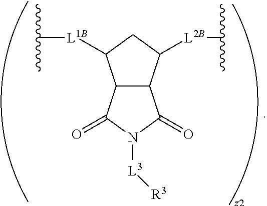

In embodiments, L^{1B} and L^{2B} are independently a bond, —O—, —S—, —NH—, —C(O)—, —C(O)O—, —C(O)NH—, substituted or unsubstituted alkylene, substituted or unsubstituted heteroalkylene.

In embodiments, L³-R³ is a hydrophobic moiety. In embodiments, L³ is a hydrophobic moiety. In embodiments, R³ is a hydrophobic moiety.

In embodiments, L³ is a bond, —O—, —S—, —NH—, —C(O)—, —C(O)O—, —C(O)NH—, R¹³-substituted or unsubstituted alkylene (e.g., C₁-C₈ alkylene, C₁-C₆ alkylene, or C₁-C₄ alkylene), R¹³-substituted or unsubstituted heteroalkylene (e.g., 2 to 8 membered heteroalkylene, 2 to 6 membered heteroalkylene, or 2 to 4 membered heteroalkylene), R¹³-substituted or unsubstituted cycloalkylene (e.g., C₃-C₈ cycloalkylene, C₃-C₆ cycloalkylene, or C₅-C₆ cycloalkylene), R¹³-substituted or unsubstituted heterocycloalkylene (e.g., 3 to 8 membered heterocycloalkylene, 3 to 6 membered heterocycloalkylene, or 5 to 6 membered heterocycloalkylene), R¹³-substituted or unsubstituted arylene (e.g., C₆-C₁₀ arylene, C₁₀ arylene, or phenylene), or R¹³-substituted or unsubstituted heteroarylene (e.g., 5 to 10 membered heteroarylene, 5 to 9 membered heteroarylene, or 5 to 6 membered heteroarylene).

In embodiments, R³ is hydrogen, R¹⁴-substituted or unsubstituted alkyl (e.g., C₁-C₈ alkyl, C₁-C₆ alkyl, or C₁-C₄ alkyl), R¹⁴-substituted or unsubstituted heteroalkyl (e.g., 2 to 8 membered heteroalkyl, 2 to 6 membered heteroalkyl, or 2 to 4 membered heteroalkyl), R¹⁴-substituted or unsubstituted cycloalkyl (e.g., C₃-C₈ cycloalkyl, C₃-C₆ cycloalkyl, or C₅-C₆ cycloalkyl), R¹⁴-substituted or unsubstituted heterocycloalkyl (e.g., 3 to 8 membered heterocycloalkyl, 3 to 6 membered heterocycloalkyl, or 5 to 6 membered heterocycloalkyl), R¹⁴-substituted or unsubstituted aryl (e.g., C₆-C₁₀ aryl, C₁₀ aryl, or phenyl), R¹⁴-substituted or unsubstituted heteroaryl (e.g., 5 to 10 membered heteroaryl, 5 to 9 membered heteroaryl, or 5 to 6 membered heteroaryl). In embodiments, R³ is hydrogen.

In embodiments, L³-R³ is

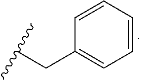 [SMILES: *Cc1ccccc1]

In embodiments, L³ is a substituted or unsubstituted C₁-C₈ alkylene and R³ is phenyl. In embodiments, L³ a substituted or unsubstituted C₁-C₆ alkylene and R³ is phenyl. In embodiments, L³ a substituted or unsubstituted C₁-C₄ alkylene and R³ is phenyl. In embodiments, L³ a substituted or unsubstituted C₁-C₂ alkylene and R³ is phenyl. In embodiments, L³ a substituted or unsubstituted methylene and R³ is phenyl. In embodiments, L³ an unsubstituted methylene and R³ is phenyl.

In embodiments, C is a second block monomer backbone moiety. Thus, in embodiments, (C(-L⁴-R⁴))_{z5} has the formula (-L^{1C}-C¹(-L⁴-R⁴)-L^{2C}-)_{z5}. L^{1C} and L^{2C} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. C¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, C¹ is R²⁹-substituted or unsubstituted alkylene (e.g., C₁-C₈ alkylene, C₁-C₆ alkylene, or C₁-C₄ alkylene), R²⁹-substituted or unsubstituted heteroalkylene (e.g., 2 to 8 membered heteroalkylene, 2 to 6 membered heteroalkylene, or 2 to 4 membered heteroalkylene), R²⁹-substituted or unsubstituted cycloalkylene (e.g., C₃-C₈ cycloalkylene, C₃-C₆ cycloalkylene, or C₅-C₆ cycloalkylene), R²⁹-substituted or unsubstituted heterocycloalkylene (e.g., 3 to 8 membered heterocycloalkylene, 3 to 6 membered heterocycloalkylene, or 5 to 6 membered heterocycloalkylene), R²⁹-substituted or unsubstituted arylene (e.g., C₆-C₁₀ arylene, C₁₀ arylene, or phenylene), or R²⁹-substituted or unsubstituted heteroarylene (e.g., 5 to 10 membered heteroarylene, 5 to 9 membered heteroarylene, or 5 to 6 membered heteroarylene).

In embodiments, C¹ is R²⁹-substituted or unsubstituted alkylene (e.g., C₁-C₈ alkylene, C₁-C₆ alkylene, or C₁-C₄ alkylene). In embodiments, C¹ is R²⁹-substituted alkylene (e.g., C₁-C₈ alkylene, C₁-C₆ alkylene, or C₁-C₄ alkylene). In embodiments, C¹ is an unsubstituted alkylene (e.g., C₁-C₈ alkylene, C₁-C₆ alkylene, or C₁-C₄ alkylene).

In embodiments, C¹ is R²⁹-substituted or unsubstituted heteroalkylene (e.g., 2 to 8 membered heteroalkylene, 2 to 6 membered heteroalkylene, or 2 to 4 membered heteroalkylene). In embodiments, C¹ is R²⁹-substituted heteroalkylene (e.g., 2 to 8 membered heteroalkylene, 2 to 6 membered heteroalkylene, or 2 to 4 membered heteroalkylene). In embodiments, C¹ is an unsubstituted heteroalkylene (e.g., 2 to 8 membered heteroalkylene, 2 to 6 membered heteroalkylene, or 2 to 4 membered heteroalkylene).

In embodiments, C¹ is R²⁹-substituted or unsubstituted cycloalkylene (e.g., C₃-C₈ cycloalkylene, C₃-C₆ cycloalkylene, or C₅-C₆ cycloalkylene). In embodiments, C₁ is R²⁹-substituted cycloalkylene (e.g., C₃-C₈ cycloalkylene, C₃-C₆ cycloalkylene, or C₅-C₆ cycloalkylene). In embodiments, C¹ is an unsubstituted cycloalkylene (e.g., C₃-C₈ cycloalkylene, C₃-C₆ cycloalkylene, or C₅-C₆ cycloalkylene).

In embodiments, C¹ is R²⁹-substituted or unsubstituted heterocycloalkylene (e.g., 3 to 8 membered heterocycloalkylene, 3 to 6 membered heterocycloalkylene, or 5 to 6 membered heterocycloalkylene). In embodiments, C¹ is R²⁹-substituted heterocycloalkylene (e.g., 3 to 8 membered heterocycloalkylene, 3 to 6 membered heterocycloalkylene, or 5 to 6 membered heterocycloalkylene). In embodiments, C¹ is an unsubstituted heterocycloalkylene (e.g., 3 to 8 membered heterocycloalkylene, 3 to 6 membered heterocycloalkylene, or 5 to 6 membered heterocycloalkylene).

In embodiments, C¹ is R²⁹-substituted or unsubstituted arylene (e.g., C₆-C₁₀ arylene, C₁₀ arylene, or phenylene). In embodiments, C¹ is R²⁹-substituted arylene (e.g., C₆-C₁₀ arylene, C₁₀ arylene, or phenylene). In embodiments, C₁ is an unsubstituted arylene (e.g., C₆-C₁₀ arylene, C₁₀ arylene, or phenylene).

In embodiments, C¹ is R²⁹-substituted or unsubstituted heteroarylene (e.g., 5 to 10 membered heteroarylene, 5 to 9 membered heteroarylene, or 5 to 6 membered heteroarylene). In embodiments, C¹ is R²⁹-substituted heteroarylene (e.g., 5 to 10 membered heteroarylene, 5 to 9 membered heteroarylene, or 5 to 6 membered heteroarylene). In embodiments, C₁ is an unsubstituted heteroarylene (e.g., 5 to 10 membered heteroarylene, 5 to 9 membered heteroarylene, or 5 to 6 membered heteroarylene).

R²⁹ is independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, R³⁰-substituted or unsubstituted alkyl (e.g., C₁-C₈ alkyl, C₁-C₆ alkyl, or C₁-C₄ alkyl), RN-substituted or unsubstituted heteroalkyl (e.g., 2 to 8 membered heteroalkyl, 2 to 6 membered heteroalkyl, or 2 to 4 membered heteroalkyl), RN-substituted or unsubstituted cycloalkyl (e.g., C₃-C₈ cycloalkyl, C₃-C₆ cycloalkyl, or C₅-C₆ cycloalkyl), RN-substituted or unsubstituted heterocycloalkyl (e.g., 3 to 8 membered heterocycloalkyl, 3 to 6 membered heterocycloalkyl, or 5 to 6 membered heterocycloalkyl), R³⁰-substituted or unsubstituted aryl (e.g., C₆-C₁₀ aryl, C₁₀ aryl, or phenyl), or R³⁰-substituted or unsubstituted heteroaryl (e.g., 5 to 10 membered heteroaryl, 5 to 9 membered heteroaryl, or 5 to 6 membered heteroaryl).

In embodiments, R²⁹ is oxo, —OH, —NH₂, —COOH, or —CONH₂. In embodiments, R²⁹ is oxo.

In embodiments, L^{1C} is a bond, —O—, —S—, —NH—, —C(O)—, —C(O)O—, —C(O)NH—, R^{22C}-substituted or unsubstituted alkylene (e.g., C₁-C₈ alkylene, C₁-C₆ alkylene, or C₁-C₄ alkylene), R^{22C}-substituted or unsubstituted heteroalkylene (e.g., 2 to 8 membered heteroalkylene, 2 to 6 membered heteroalkylene, or 2 to 4 membered heteroalkylene), R^{22C}-substituted or unsubstituted cycloalkylene (e.g., C₃-C₈ cycloalkylene, C₃-C₆ cycloalkylene, or C₅-C₆ cycloalkylene), R^{22C}-substituted or unsubstituted heterocycloalkylene (e.g., 3 to 8 membered heterocycloalkylene, 3 to 6 membered heterocycloalkylene, or 5 to 6 membered heterocycloalkylene), R^{22C}-substituted or unsubstituted arylene (e.g., C₆-C₁₀ arylene, C₁₀ arylene, or phenylene), or R^{22C}-substituted or unsubstituted heteroarylene (e.g., 5 to 10 membered heteroarylene, 5 to 9 membered heteroarylene, or 5 to 6 membered heteroarylene). In embodiments, L^{1C} is a bond, —O—, —S—, —NH—, —C(O)—, —C(O)O—, —C(O)NH—, R^{22C}-substituted or unsubstituted alkenylene (e.g., C₂-C₈ alkenylene, C₂-C₆ alkenylene, or C₂-C₄ alkenylene). In embodiments, L^{1C} is a substituted or unsubstituted ethylenyl.

In embodiments, L²c is a bond, —O—, —S—, —NH—, —C(O)—, —C(O)O—, —C(O)NH—, R^{23C}-substituted or unsubstituted alkylene (e.g., C₁-C₈ alkylene, C₁-C₆ alkylene, or C₁-C₄ alkylene), R^{23C}-substituted or unsubstituted heteroalkylene (e.g., 2 to 8 membered heteroalkylene, 2 to 6 membered heteroalkylene, or 2 to 4 membered heteroalkylene), R^{23C}-substituted or unsubstituted cycloalkylene (e.g., C₃-C₈ cycloalkylene, C₃-C₆ cycloalkylene, or C₅-C₆ cycloalkylene), R^{23C}-substituted or unsubstituted heterocycloalkylene (e.g., 3 to 8 membered heterocycloalkylene, 3 to 6 membered heterocycloalkylene, or 5 to 6 membered heterocycloalkylene), R^{23C}-substituted or unsubstituted arylene (e.g., C₆-C₁₀ arylene, C₁₀ arylene, or phenylene), or R^{23C}-substituted or unsubstituted heteroarylene (e.g., 5 to 10 membered heteroarylene, 5 to 9 membered heteroarylene, or 5 to 6 membered heteroarylene). In embodiments, L^{2C} is a bond, —O—, —S—, —NH—, —C(O)—, —C(O)O—, —C(O)NH—, R^{23C}-substituted or unsubstituted alkenylene (e.g., C₂-C₈ alkenylene, C₂-C₆ alkenylene, or C₂-C₄ alkenylene). In embodiments, L^{2C} is a substituted or unsubstituted ethylenyl.

In embodiments, (C(-L⁴-R⁴))_{z5} has the formula:

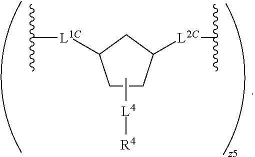

In embodiments, L^{1C} and L^{2C} are independently a bond, —O—, —S—, —NH—, —C(O)—, —C(O)O—, —C(O)NH—, substituted or unsubstituted alkylene, substituted or unsubstituted heteroalkylene.

In embodiments, (C(-L⁴-R⁴))_{z7} has the formula (-L^{1C}-C¹)-L⁴-R⁴)-L^{2C}-)_{z7}. In embodiments, (C(-L⁴-R⁴))_{z7} has the formula:

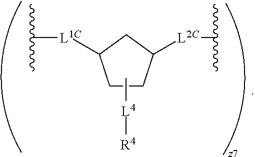

In embodiments, L^{1C} and L^{2C} are independently a bond, —O—, —S—, —NH—, —C(O)—, —C(O)O—, —C(O)NH—, substituted or unsubstituted alkylene, substituted or unsubstituted heteroalkylene.

In embodiments, L⁴-R⁴ is a hydrophilic moiety. In embodiments, L⁴ is a hydrophilic moiety. In embodiments, R⁴ is a hydrophilic moiety.

In embodiments, L⁴ is a bond, —O—, —S—, —NH—, —C(O)—, —C(O)O—, —C(O)NH—, R¹⁵-substituted or unsubstituted alkylene (e.g., C₁-C₈ alkylene, C₁-C₆ alkylene, or C₁-C₄ alkylene), R¹⁵-substituted or unsubstituted heteroalkylene (e.g., 2 to 8 membered heteroalkylene, 2 to 6 membered heteroalkylene, or 2 to 4 membered heteroalkylene), R¹⁵-substituted or unsubstituted cycloalkylene (e.g., C₃-C₈ cycloalkylene, C₃-C₆ cycloalkylene, or C₅-C₆ cycloalkylene), R¹⁵-substituted or unsubstituted heterocycloalkylene (e.g., 3 to 8 membered heterocycloalkylene, 3 to 6 membered heterocycloalkylene, or 5 to 6 membered heterocycloalkylene), R¹⁵-substituted or unsubstituted arylene (e.g., C₆-C₁₀ arylene, C₁₀ arylene, or phenylene), or R¹⁵-substituted or unsubstituted heteroarylene (e.g., 5 to 10 membered heteroarylene, 5 to 9 membered heteroarylene, or 5 to 6 membered heteroarylene).

In embodiments, R⁴ is hydrogen, R¹⁶-substituted or unsubstituted alkyl (e.g., C₁-C₈ alkyl, C₁-C₆ alkyl, or C₁-C₄ alkyl), R¹⁶-substituted or unsubstituted heteroalkyl (e.g., 2 to 8 membered heteroalkyl, 2 to 6 membered heteroalkyl, or 2 to 4 membered heteroalkyl), R¹⁶-substituted or unsubstituted cycloalkyl (e.g., C₃-C₈ cycloalkyl, C₃-C₆ cycloalkyl, or C₅-C₆ cycloalkyl), R¹⁶-substituted or unsubstituted heterocycloalkyl (e.g., 3 to 8 membered heterocycloalkyl, 3 to 6 membered heterocycloalkyl, or 5 to 6 membered heterocycloalkyl), R¹⁶-substituted or unsubstituted aryl (e.g., C₆-C₁₀ aryl, C₁₀ aryl, or phenyl), R¹⁶-substituted or unsubstituted heteroaryl (e.g., 5 to 10 membered heteroaryl, 5 to 9 membered heteroaryl, or 5 to 6 membered heteroaryl) a hydrophobic drug moiety, a targeting moiety or a detectable moiety.

In embodiments, L⁴-R⁴ is —C(O)OH. In embodiments, L⁴ is a bond and R⁴ is —C(O)OH. In embodiments, L⁴ is a bond. In embodiments, R⁴ is —C(O)OH.

In embodiments, D is a second block monomer backbone moiety. Thus, in embodiments, (D(-L⁵-R⁵))_{z6} has the formula (-L^{1D}-D¹(-L⁵-R⁵)-L^{2D}-)_{z6}. L^{1D} and L^{2D} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. D¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, D¹ is R³¹-substituted or unsubstituted alkylene (e.g., C₁-C₈ alkylene, C₁-C₆ alkylene, or C₁-C₄ alkylene), R³¹-substituted or unsubstituted heteroalkylene (e.g., 2 to 8 membered heteroalkylene, 2 to 6 membered heteroalkylene, or 2 to 4 membered heteroalkylene), R³¹-substituted or unsubstituted cycloalkylene (e.g., C₃-C₈ cycloalkylene, C₃-C₆ cycloalkylene, or C₅-C₆ cycloalkylene), R³¹-substituted or unsubstituted heterocycloalkylene (e.g., 3 to 8 membered heterocycloalkylene, 3 to 6 membered heterocycloalkylene, or 5 to 6 membered heterocycloalkylene), R³¹-substituted or unsubstituted arylene (e.g., C₆-C₁₀ arylene, C₁₀ arylene, or phenylene), or R³¹-substituted or unsubstituted heteroarylene (e.g., 5 to 10 membered heteroarylene, 5 to 9 membered heteroarylene, or 5 to 6 membered heteroarylene).

In embodiments, D¹ is R³¹-substituted or unsubstituted alkylene (e.g., C₁-C₈ alkylene, C₁-C₆ alkylene, or C₁-C₄ alkylene). In embodiments, D¹ is R³¹-substituted alkylene (e.g., C₁-C₈ alkylene, C₁-C₆ alkylene, or C₁-C₄ alkylene). In embodiments, D¹ is an unsubstituted alkylene (e.g., C₁-C₈ alkylene, C₁-C₆ alkylene, or C₁-C₄ alkylene).

In embodiments, D¹ is R³¹-substituted or unsubstituted heteroalkylene (e.g., 2 to 8 membered heteroalkylene, 2 to 6 membered heteroalkylene, or 2 to 4 membered heteroalkylene). In embodiments, D¹ is R³¹-substituted heteroalkylene (e.g., 2 to 8 membered heteroalkylene, 2 to 6 membered heteroalkylene, or 2 to 4 membered heteroalkylene). In embodiments, D¹ is an unsubstituted heteroalkylene (e.g., 2 to 8 membered heteroalkylene, 2 to 6 membered heteroalkylene, or 2 to 4 membered heteroalkylene).

In embodiments, D¹ is R³¹-substituted or unsubstituted cycloalkylene (e.g., C₃-C₈ cycloalkylene, C₃-C₆ cycloalkylene, or C₅-C₆ cycloalkylene). In embodiments, D¹ is R³¹-substituted cycloalkylene (e.g., C₃-C₈ cycloalkylene, C₃-C₆ cycloalkylene, or C₅-C₆ cycloalkylene). In embodiments, D¹ is an unsubstituted cycloalkylene (e.g., C₃-C₈ cycloalkylene, C₃-C₆ cycloalkylene, or C₅-C₆ cycloalkylene).

In embodiments, D¹ is R³¹-substituted or unsubstituted heterocycloalkylene (e.g., 3 to 8 membered heterocycloalkylene, 3 to 6 membered heterocycloalkylene, or 5 to 6 membered heterocycloalkylene). In embodiments, D¹ is R³¹-substituted heterocycloalkylene (e.g., 3 to 8 membered heterocycloalkylene, 3 to 6 membered heterocycloalkylene, or 5 to 6 membered heterocycloalkylene). In embodiments, D¹ is an unsubstituted heterocycloalkylene (e.g., 3 to 8 membered heterocycloalkylene, 3 to 6 membered heterocycloalkylene, or 5 to 6 membered heterocycloalkylene).

In embodiments, D¹ is R³¹-substituted or unsubstituted arylene (e.g., C₆-C₁₀ arylene, C₁₀ arylene, or phenylene). In embodiments, D¹ is R³¹-substituted arylene (e.g., C₆-C₁₀ arylene, C₁₀ arylene, or phenylene). In embodiments, D¹ is an unsubstituted arylene (e.g., C₆-C₁₀ arylene, C₁₀ arylene, or phenylene).

In embodiments, D¹ is R³¹-substituted or unsubstituted heteroarylene (e.g., 5 to 10 membered heteroarylene, 5 to 9 membered heteroarylene, or 5 to 6 membered heteroarylene). In embodiments, D¹ is R³¹-substituted heteroarylene (e.g., 5 to 10 membered heteroarylene, 5 to 9 membered heteroarylene, or 5 to 6 membered heteroarylene). In embodiments, D¹ is an unsubstituted heteroarylene (e.g., 5 to 10 membered heteroarylene, 5 to 9 membered heteroarylene, or 5 to 6 membered heteroarylene).

R³¹ is independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, R³²-substituted or unsubstituted alkyl (e.g., C₁-C₈ alkyl, C₁-C₆ alkyl, or C₁-C₄ alkyl), R³²-substituted or unsubstituted heteroalkyl (e.g., 2 to 8 membered heteroalkyl, 2 to 6 membered heteroalkyl, or 2 to 4 membered heteroalkyl), R³²-substituted or unsubstituted cycloalkyl (e.g., C₃-C₈ cycloalkyl, C₃-C₆ cycloalkyl, or C₅-C₆ cycloalkyl), R³²-substituted or unsubstituted heterocycloalkyl (e.g., 3 to 8 membered heterocycloalkyl, 3 to 6 membered heterocycloalkyl, or 5 to 6 membered heterocycloalkyl), R³²-substituted or unsubstituted aryl (e.g., C₆-C₁₀ aryl, C₁₀ aryl, or phenyl), or R³²-substituted or unsubstituted heteroaryl (e.g., 5 to 10 membered heteroaryl, 5 to 9 membered heteroaryl, or 5 to 6 membered heteroaryl).

In embodiments, R³¹ is oxo, —OH, —NH₂, —COOH, or —CONH₂. In embodiments, R³¹ is oxo.

In embodiments, L^{1D} is a bond, —O—, —S—, —NH—, —C(O)—, —C(O)O—, —C(O)NH—, R^{22D}-substituted or unsubstituted alkylene (e.g., C₁-C₈ alkylene, C₁-C₆ alkylene, or C₁-C₄ alkylene), R^{22D}-substituted or unsubstituted heteroalkylene (e.g., 2 to 8 membered heteroalkylene, 2 to 6 membered heteroalkylene, or 2 to 4 membered heteroalkylene), R^{22D}-substituted or unsubstituted cycloalkylene (e.g., C₃-C₈ cycloalkylene, C₃-C₆ cycloalkylene, or C₅-C₆ cycloalkylene), R^{22D}-substituted or unsubstituted heterocycloalkylene (e.g., 3 to 8 membered heterocycloalkylene, 3 to 6 membered heterocycloalkylene, or 5 to 6 membered heterocycloalkylene), R^{22D}-substituted or unsubstituted arylene (e.g., C₆-C₁₀ arylene, C₁₀ arylene, or phenylene), or R^{22D}-substituted or unsubstituted heteroarylene (e.g., 5 to 10 membered heteroarylene, 5 to 9 membered heteroarylene, or 5 to 6 membered heteroarylene). In embodiments, L^{1D} is a bond, —O—, —S—, —NH—, —C(O)—, —C(O)O—, —C(O)NH—, R^{22D}-substituted or unsubstituted alkenylene (e.g., C₂-C₈ alkenylene, C₂-C₆ alkenylene, or C₂-C₄ alkenylene). In embodiments, L^{1D} is a substituted or unsubstituted ethylenyl.

In embodiments, L^{2D} is a bond, —O—, —S—, —NH—, —C(O)—, —C(O)O—, —C(O)NH—, R^{23D}-substituted or unsubstituted alkylene (e.g., C₁-C₈ alkylene, C₁-C₆ alkylene, or C₁-C₄ alkylene), R^{23D}-substituted or unsubstituted heteroalkylene (e.g., 2 to 8 membered heteroalkylene, 2 to 6 membered heteroalkylene, or 2 to 4 membered heteroalkylene), R^{23D}-substituted or unsubstituted cycloalkylene (e.g., C₃-C₈ cycloalkylene, C₃-C₆ cycloalkylene, or C₅-C₆ cycloalkylene), R^{23D}-substituted or unsubstituted heterocycloalkylene (e.g., 3 to 8 membered heterocycloalkylene, 3 to 6 membered heterocycloalkylene, or 5 to 6 membered heterocycloalkylene), R^{23D}-substituted or unsubstituted arylene (e.g., C₆-C₁₀ arylene, C₁₀ arylene, or phenylene), or R^{23D}-substituted or unsubstituted heteroarylene (e.g., 5 to 10 membered heteroarylene, 5 to 9 membered heteroarylene, or 5 to 6 membered heteroarylene). In embodiments, L^{2D} is a bond, —O—, —S—, —NH—, —C(O)—, —C(O)O—, —C(O)NH—, R^{23D}-substituted or unsubstituted alkenylene (e.g., C₂-C₈ alkenylene, C₂-C₆ alkenylene, or C₂-C₄ alkenylene). In embodiments, L^{2D} is a substituted or unsubstituted ethylenyl.

In embodiments, L⁵-R⁵ is a hydrophilic moiety. In embodiments, L⁵ is a hydrophilic moiety. In embodiments, R⁵ is a hydrophilic moiety. In embodiments, R⁵ is a peptide which is a hydrophilic moiety. In embodiments, R⁵ is a peptide which is a hydrophilic moiety that contains an inflammatory protease (e.g., cardiovascular inflammatory protease, myocardial inflammatory protease, MMP-2, or MMP9) cleavable amino acid sequence.

In embodiments, L⁵ is a bond, —O—, —S—, —NH—, —C(O)—, —C(O)O—, —C(O)NH—, R¹⁷-substituted or unsubstituted alkylene (e.g., C₁-C₈ alkylene, C₁-C₆ alkylene, or C₁-C₄ alkylene), R¹⁷-substituted or unsubstituted heteroalkylene (e.g., 2 to 8 membered heteroalkylene, 2 to 6 membered heteroalkylene, or 2 to 4 membered heteroalkylene), R¹⁷-substituted or unsubstituted cycloalkylene (e.g., C₃-C₈ cycloalkylene, C₃-C₆ cycloalkylene, or C₅-C₆ cycloalkylene), R¹⁷-substituted or unsubstituted heterocycloalkylene (e.g., 3 to 8 membered heterocycloalkylene, 3 to 6 membered heterocycloalkylene, or 5 to 6 membered heterocycloalkylene), R¹⁷-substituted or unsubstituted arylene (e.g., C₆-C₁₀ arylene, C₁₀ arylene, or phenylene), or R¹⁷-substituted or unsubstituted heteroarylene (e.g., 5 to 10 membered heteroarylene, 5 to 9 membered heteroarylene, or 5 to 6 membered heteroarylene).

In embodiments, L⁵ is a bond, —O—, —S—, —NH—, —C(O)—, —C(O)O—, —C(O)NH—. In embodiments, L⁵ is —C(O)—, —C(O)O—, —C(O)NH—. In embodiments, L⁵ is —C(O)NH—.

In embodiments, R⁵ is an amino acid sequence comprising an MMP-9 or MMP-2 cleavable amino acid sequence. In embodiments, R⁵ is an amino acid sequence comprising an MMP-9 cleavable amino acid sequence. In embodiments, R⁵ is an amino acid sequence comprising an MMP-2 cleavable amino acid sequence.

In embodiments, (D(-L⁵-R⁵))_{z6} has the formula:

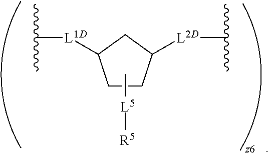

In embodiments, L^{1D} and L^{2D} are independently a bond, —O—, —S—, —NH—, —C(O)—, —C(O)O—, —C(O)NH—, substituted or unsubstituted alkylene, substituted or unsubstituted heteroalkylene.

In embodiments, (D(L⁵-R⁵))_{z6} has the formula:

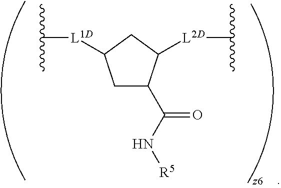

In embodiments, L^{1D} and L^{2D} are independently a bond, —O—, —S—, —NH—, —C(O)—, —C(O)O—, —C(O)NH—, substituted or unsubstituted alkylene, substituted or unsubstituted heteroalkylene.

In embodiments, L⁶ is a bond, —O—, —S—, —NH—, —C(O)—, —C(O)O—, —C(O)NH—, -L^{6A}-L^{6B}-L^{6C}-, R¹⁸-substituted or unsubstituted alkylene (e.g., C₁-C₈ alkylene, C₁-C₆ alkylene, or C₁-C₄ alkylene), R¹⁸-substituted or unsubstituted heteroalkylene (e.g., 2 to 8 membered heteroalkylene, 2 to 6 membered heteroalkylene, or 2 to 4 membered heteroalkylene), R¹⁸-substituted or unsubstituted cycloalkylene (e.g., C₃-C₈ cycloalkylene, C₃-C₆ cycloalkylene, or C₅-C₆ cycloalkylene), R¹⁸-substituted or unsubstituted heterocycloalkylene (e.g., 3 to 8 membered heterocycloalkylene, 3 to 6 membered heterocycloalkylene, or 5 to 6 membered heterocycloalkylene), R¹⁸-substituted or unsubstituted arylene (e.g., C₆-C₁₀ arylene, C₁₀ arylene, or phenylene), or R¹⁸-substituted or unsubstituted heteroarylene (e.g., 5 to 10 membered heteroarylene, 5 to 9 membered heteroarylene, or 5 to 6 membered heteroarylene). L^{6A}, L^{6B} and L^{6C} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L⁶ is R¹⁸-substituted or unsubstituted alkylene (e.g., C₁-C₈ alkylene, C₁-C₆ alkylene, or C₁-C₄ alkylene). In embodiments, L⁶ is R¹⁸-substituted alkylene (e.g., C₁-C₈ alkylene, C₁-C₆ alkylene, or C₁-C₄ alkylene). In embodiments, L⁶ is an unsubstituted alkylene (e.g., C₁-C₈ alkylene, C₁-C₆ alkylene, or C₁-C₄ alkylene).

In embodiments, L⁶ is R¹⁸-substituted or unsubstituted heteroalkylene (e.g., 2 to 8 membered heteroalkylene, 2 to 6 membered heteroalkylene, or 2 to 4 membered heteroalkylene). In embodiments, L⁶ is R¹⁸-substituted heteroalkylene (e.g., 2 to 8 membered heteroalkylene, 2 to 6 membered heteroalkylene, or 2 to 4 membered heteroalkylene). In embodiments, L⁶ is an unsubstituted heteroalkylene (e.g., 2 to 8 membered heteroalkylene, 2 to 6 membered heteroalkylene, or 2 to 4 membered heteroalkylene).

In embodiments, L⁶ is R¹⁸-substituted or unsubstituted cycloalkylene (e.g., C₃-C₈ cycloalkylene, C₃-C₆ cycloalkylene, or C₅-C₆ cycloalkylene). In embodiments, L⁶ is R¹⁸-substituted cycloalkylene (e.g., C₃-C₈ cycloalkylene, C₃-C₆ cycloalkylene, or C₅-C₆ cycloalkylene). In embodiments, L⁶ is an unsubstituted cycloalkylene (e.g., C₃-C₈ cycloalkylene, C₃-C₆ cycloalkylene, or C₅-C₆ cycloalkylene).

In embodiments, L⁶ is R¹⁸-substituted or unsubstituted heterocycloalkylene (e.g., 3 to 8 membered heterocycloalkylene, 3 to 6 membered heterocycloalkylene, or 5 to 6 membered heterocycloalkylene). In embodiments, L⁶ is R¹⁸-substituted heterocycloalkylene (e.g., 3 to 8 membered heterocycloalkylene, 3 to 6 membered heterocycloalkylene, or 5 to 6 membered heterocycloalkylene). In embodiments, L⁶ is an unsubstituted heterocycloalkylene (e.g., 3 to 8 membered heterocycloalkylene, 3 to 6 membered heterocycloalkylene, or 5 to 6 membered heterocycloalkylene).

In embodiments, L⁶ is R¹⁸-substituted or unsubstituted arylene (e.g., C₆-C₁₀ arylene, C₁₀ arylene, or phenylene). In embodiments, L⁶ is R¹⁸-substituted arylene (e.g., C₆-C₁₀ arylene, C₁₀ arylene, or phenylene). In embodiments, L⁶ is an unsubstituted arylene (e.g., C₆-C₁₀ arylene, C₁₀ arylene, or phenylene).

In embodiments, L⁶ is R¹⁸-substituted or unsubstituted heteroarylene (e.g., 5 to 10 membered heteroarylene, 5 to 9 membered heteroarylene, or 5 to 6 membered heteroarylene). In embodiments, L⁶ is R¹⁸-substituted heteroarylene (e.g., 5 to 10 membered heteroarylene, 5 to 9 membered heteroarylene, or 5 to 6 membered heteroarylene). In embodiments, L⁶ is an unsubstituted heteroarylene (e.g., 5 to 10 membered heteroarylene, 5 to 9 membered heteroarylene, or 5 to 6 membered heteroarylene).

R¹⁸ is independently oxo,

halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, R¹⁹-substituted or unsubstituted alkyl (e.g., C₁-C₈ alkyl, C₁-C₆ alkyl, or C₁-C₄ alkyl), R¹⁹-substituted or unsubstituted heteroalkyl (e.g., 2 to 8 membered heteroalkyl, 2 to 6 membered heteroalkyl, or 2 to 4 membered heteroalkyl), R¹⁹-substituted or unsubstituted cycloalkyl (e.g., C₃-C₈ cycloalkyl, C₃-C₆ cycloalkyl, or C₅-C₆ cycloalkyl), R¹⁹-substituted or unsubstituted heterocycloalkyl (e.g., 3 to 8 membered heterocycloalkyl, 3 to 6 membered heterocycloalkyl, or 5 to 6 membered heterocycloalkyl), R¹⁹-substituted or unsubstituted aryl (e.g., C₆-C₁₀ aryl, C₁₀ aryl, or phenyl), or R¹⁹-substituted or unsubstituted heteroaryl (e.g., 5 to 10 membered heteroaryl, 5 to 9 membered heteroaryl, or 5 to 6 membered heteroaryl).

In embodiments, L⁶ is -L^{6A}-L^{6B}-L^{6C}-. In embodiments, L^{6A}, L^{6B} and L^{6C} are independently a bond substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L^{6A} is R^{18A}-substituted or unsubstituted alkylene (e.g., C₁-C₈ alkylene, C₁-C₆ alkylene, or C₁-C₄ alkylene). In embodiments, L^{6A} is R^{18A}-substituted alkylene (e.g., C₁-C₈ alkylene, C₁-C₆ alkylene, or C₁-C₄ alkylene). In embodiments, L^{6A} is an unsubstituted alkylene (e.g., C₁-C₈ alkylene, C₁-C₆ alkylene, or C₁-C₄ alkylene). In embodiments, L^{6A} is R^{18A}-substituted or unsubstituted heteroalkylene (e.g., 2 to 8 membered heteroalkylene, 2 to 6 membered heteroalkylene, or 2 to 4 membered heteroalkylene). In embodiments, L^{6A} is R^{18A}-substituted heteroalkylene (e.g., 2 to 8 membered heteroalkylene, 2 to 6 membered heteroalkylene, or 2 to 4 membered heteroalkylene). In embodiments, L^{6A} is an unsubstituted heteroalkylene (e.g., 2 to 8 membered heteroalkylene, 2 to 6 membered heteroalkylene, or 2 to 4 membered heteroalkylene). In embodiments, L^{6A} is R^{18A}-substituted or unsubstituted cycloalkylene (e.g., C₃-C₈ cycloalkylene, C₃-C₆ cycloalkylene, or C₅-C₆ cycloalkylene). In embodiments, L^{6A} is R^{18A}-substituted cycloalkylene (e.g., C₃-C₈ cycloalkylene, C₃-C₆ cycloalkylene, or C₅-C₆ cycloalkylene). In embodiments, L^{6A} is an unsubstituted cycloalkylene (e.g., C₃-C₈ cycloalkylene, C₃-C₆ cycloalkylene, or C₅-C₆ cycloalkylene). In embodiments, L^{6A} is R^{18A}-substituted or unsubstituted heterocycloalkylene (e.g., 3 to 8 membered heterocycloalkylene, 3 to 6 membered heterocycloalkylene, or 5 to 6 membered heterocycloalkylene). In embodiments, L^{6A} is R^{18A}-substituted heterocycloalkylene (e.g., 3 to 8 membered heterocycloalkylene, 3 to 6 membered heterocycloalkylene, or 5 to 6 membered heterocycloalkylene). In embodiments, L^{6A} is an unsubstituted heterocycloalkylene (e.g., 3 to 8 membered heterocycloalkylene, 3 to 6 membered heterocycloalkylene, or 5 to 6 membered heterocycloalkylene). In embodiments, L^{6A} is R^{18A}-substituted or unsubstituted arylene (e.g., C₆-C₁₀ arylene, C₁₀ arylene, or phenylene). In embodiments, L^{6A} is R^{18A}-substituted arylene (e.g., C₆-C₁₀ arylene, C₁₀ arylene, or phenylene). In embodiments, L^{6A} is an unsubstituted arylene (e.g., C₆-C₁₀ arylene, C₁₀ arylene, or phenylene). In embodiments, L^{6A} is R^{18A}-substituted or unsubstituted heteroarylene (e.g., 5 to 10 membered heteroarylene, 5 to 9 membered heteroarylene, or 5 to 6 membered heteroarylene). In embodiments, L^{6A} is R^{18A}-substituted heteroarylene (e.g., 5 to 10 membered heteroarylene, 5 to 9 membered heteroarylene, or 5 to 6 membered heteroarylene). In embodiments, L^{6A} is an unsubstituted heteroarylene (e.g., 5 to 10 membered heteroarylene, 5 to 9 membered heteroarylene, or 5 to 6 membered heteroarylene).

In embodiments, L^{6B} is R^{18B}-substituted or unsubstituted alkylene (e.g., C₁-C₈ alkylene, C₁-C₆ alkylene, or C₁-C₄ alkylene). In embodiments, L^{6B} is R^{18B}-substituted alkylene (e.g., C₁-C₈ alkylene, C₁-C₆ alkylene, or C₁-C₄ alkylene). In embodiments, L^{6B} is an unsubstituted alkylene (e.g., C₁-C₈ alkylene, C₁-C₆ alkylene, or C₁-C₄ alkylene). In embodiments, L^{6B} is R^{18B}-substituted or unsubstituted heteroalkylene (e.g., 2 to 8 membered heteroalkylene, 2 to 6 membered heteroalkylene, or 2 to 4 membered heteroalkylene). In embodiments, L^{6B} is R^{18B}-substituted heteroalkylene (e.g., 2 to 8 membered heteroalkylene, 2 to 6 membered heteroalkylene, or 2 to 4 membered heteroalkylene). In embodiments, L^{6B} is an unsubstituted heteroalkylene (e.g., 2 to 8 membered heteroalkylene, 2 to 6 membered heteroalkylene, or 2 to 4 membered heteroalkylene). In embodiments, L^{6B} is R^{18B}-substituted or unsubstituted cycloalkylene (e.g., C₃-C₈ cycloalkylene, C₃-C₆ cycloalkylene, or C₅-C₆ cycloalkylene). In embodiments, L^{6B} is R^{18B}-substituted cycloalkylene (e.g., C₃-C₈ cycloalkylene, C₃-C₆ cycloalkylene, or C₅-C₆ cycloalkylene). In embodiments, L^{6B} is an unsubstituted cycloalkylene (e.g., C₃-C₈ cycloalkylene, C₃-C₆ cycloalkylene, or C₅-C₆ cycloalkylene). In embodiments, L⁶B is R^{18B}-substituted or unsubstituted heterocycloalkylene (e.g., 3 to 8 membered heterocycloalkylene, 3 to 6 membered heterocycloalkylene, or 5 to 6 membered heterocycloalkylene). In embodiments, L^{6B} is R^{18B}-substituted heterocycloalkylene (e.g., 3 to 8 membered heterocycloalkylene, 3 to 6 membered heterocycloalkylene, or 5 to 6 membered heterocycloalkylene). In embodiments, L^{6B} is an unsubstituted heterocycloalkylene (e.g., 3 to 8 membered heterocycloalkylene, 3 to 6 membered heterocycloalkylene, or 5 to 6 membered heterocycloalkylene). In embodiments, L^{6B} is R^{18B}-substituted or unsubstituted arylene (e.g., C₆-C₁₀ arylene, C₁₀ arylene, or phenylene). In embodiments, L^{6B} is R^{18B}-substituted arylene (e.g., C₆-C₁₀ arylene, C₁₀ arylene, or phenylene). In embodiments, L^{6B} is an unsubstituted arylene (e.g., C₆-C₁₀ arylene, C₁₀ arylene, or phenylene). In embodiments, L^{6B} is R^{18B}-substituted or unsubstituted heteroarylene (e.g., 5 to 10 membered heteroarylene, 5 to 9 membered heteroarylene, or 5 to 6 membered heteroarylene). In embodiments, L^{6B} is R^{18B}-substituted heteroarylene (e.g., 5 to 10 membered heteroarylene, 5 to 9 membered heteroarylene, or 5 to 6 membered heteroarylene). In embodiments, L^{6B} is an unsubstituted heteroarylene (e.g., 5 to 10 membered heteroarylene, 5 to 9 membered heteroarylene, or 5 to 6 membered heteroarylene).

In embodiments, L^{6C} is R^{18C}-substituted or unsubstituted alkylene (e.g., C₁-C₈ alkylene, C₁-C₆ alkylene, or C₁-C₄ alkylene). In embodiments, L^{6C} is R^{18C}-substituted alkylene (e.g., C₁-C₈ alkylene, C₁-C₆ alkylene, or C₁-C₄ alkylene). In embodiments, L^{6C} is an unsubstituted alkylene (e.g., C₁-C₈ alkylene, C₁-C₆ alkylene, or C₁-C₄ alkylene). In embodiments, L^{6C} is R^{18C}-substituted or unsubstituted heteroalkylene (e.g., 2 to 8 membered heteroalkylene, 2 to 6 membered heteroalkylene, or 2 to 4 membered heteroalkylene). In embodiments, L^{6C} is R^{18C}-substituted heteroalkylene (e.g., 2 to 8 membered heteroalkylene, 2 to 6 membered heteroalkylene, or 2 to 4 membered heteroalkylene). In embodiments, L^{6C} is an unsubstituted heteroalkylene (e.g., 2 to 8 membered heteroalkylene, 2 to 6 membered heteroalkylene, or 2 to 4 membered heteroalkylene). In embodiments, L^{6C} is R^{18C}-substituted or unsubstituted cycloalkylene (e.g., C₃-C₈ cycloalkylene, C₃-C₆ cycloalkylene, or C₅-C₆ cycloalkylene). In embodiments, L^{6C} is R^{18C}-substituted cycloalkylene (e.g., C₃-C₈ cycloalkylene, C₃-C₆ cycloalkylene, or C₅-C₆ cycloalkylene). In embodiments, L^{6C} is an unsubstituted cycloalkylene (e.g., C₃-C₈ cycloalkylene, C₃-C₆ cycloalkylene, or C₅-C₆ cycloalkylene). In embodiments, L^{6C} is R^{18C}-substituted or unsubstituted heterocycloalkylene (e.g., 3 to 8 membered heterocycloalkylene, 3 to 6 membered heterocycloalkylene, or 5 to 6 membered heterocycloalkylene). In embodiments, L^{6C} is R^{18C}-substituted heterocycloalkylene (e.g., 3 to 8 membered heterocycloalkylene, 3 to 6 membered heterocycloalkylene, or 5 to 6 membered heterocycloalkylene). In embodiments, L^{6C} is an unsubstituted heterocycloalkylene (e.g., 3 to 8 membered heterocycloalkylene, 3 to 6 membered heterocycloalkylene, or 5 to 6 membered heterocycloalkylene). In embodiments, L^{6C} is R^{18C}-substituted or unsubstituted arylene (e.g., C₆-C₁₀ arylene, C₁₀ arylene, or phenylene). In embodiments, L^{6C} is R^{18C}-substituted arylene (e.g., C₆-C₁₀ arylene, C₁₀ arylene, or phenylene). In embodiments, L^{6C} is an unsubstituted arylene (e.g., C₆-C₁₀ arylene, C₁₀ arylene, or phenylene). In embodiments, L^{6C} is R^{18C}-substituted or unsubstituted heteroarylene (e.g., 5 to 10 membered heteroarylene, 5 to 9 membered heteroarylene, or 5 to 6 membered heteroarylene). In embodiments, L^{6C} is R^{18C}-substituted heteroarylene (e.g., 5 to 10 membered heteroarylene, 5 to 9 membered heteroarylene, or 5 to 6 membered heteroarylene). In embodiments, L^{6C} is an unsubstituted heteroarylene (e.g., 5 to 10 membered heteroarylene, 5 to 9 membered heteroarylene, or 5 to 6 membered heteroarylene).

In embodiments, L^{6A} is R^{18A}-substituted or unsubstituted alkylene (e.g., C₁-C₈ alkylene, C₁-C₆ alkylene, or C₁-C₄ alkylene), or R^{18A}-substituted or unsubstituted heteroalkylene (e.g., 2 to 8 membered heteroalkylene, 2 to 6 membered heteroalkylene, or 2 to 4 membered heteroalkylene); L^{6B} is R^{18B}-substituted or unsubstituted cycloalkylene (e.g., C₃-C₈ cycloalkylene, C₃-C₆ cycloalkylene, or C₅-C₆ cycloalkylene), R^{18B}-substituted or unsubstituted heterocycloalkylene (e.g., 3 to 8 membered heterocycloalkylene, 3 to 6 membered heterocycloalkylene, or 5 to 6 membered heterocycloalkylene, R¹⁸B-substituted or unsubstituted arylene (e.g., C₆-C₁₀ arylene, C₁₀ arylene, or phenylene), or R^{18B}-substituted or unsubstituted heteroarylene (e.g., 5 to 10 membered heteroarylene, 5 to 9 membered heteroarylene, or 5 to 6 membered heteroarylene); and L⁶ is R^{18C}-substituted or unsubstituted alkylene (e.g., C₁-C₈ alkylene, C₁-C₆ alkylene, or C₁-C₄ alkylene), or R^{18C}-substituted or unsubstituted heteroalkylene (e.g., 2 to 8 membered heteroalkylene, 2 to 6 membered heteroalkylene, or 2 to 4 membered heteroalkylene).

In embodiments, L^{6A} is R^{18A}-substituted alkylene (e.g., C₁-C₈ alkylene, C₁-C₆ alkylene, or C₁-C₄ alkylene), or R^{18A}-substituted heteroalkylene (e.g., 2 to 8 membered heteroalkylene, 2 to 6 membered heteroalkylene, or 2 to 4 membered heteroalkylene); L^{6B} is R^{18B}-substituted cycloalkylene (e.g., C₃-C₈ cycloalkylene, C₃-C₆ cycloalkylene, or C₅-C₆ cycloalkylene), R^{18B}-substituted heterocycloalkylene (e.g., 3 to 8 membered heterocycloalkylene, 3 to 6 membered heterocycloalkylene, or 5 to 6 membered heterocycloalkylene, R^{18B}-substituted arylene (e.g., C₆-C₁₀ arylene, C₁₀ arylene, or phenylene), or R^{18B}-substituted heteroarylene (e.g., 5 to 10 membered heteroarylene, 5 to 9 membered heteroarylene, or 5 to 6 membered heteroarylene); and L⁶ is R^{18C}-substituted alkylene (e.g., C₁-C₈ alkylene, C₁-C₆ alkylene, or C₁-C₄ alkylene), or R^{18C}-substituted heteroalkylene (e.g., 2 to 8 membered heteroalkylene, 2 to 6 membered heteroalkylene, or 2 to 4 membered heteroalkylene).

In embodiments, L^{6A} is an unsubstituted alkylene (e.g., C₁-C₈ alkylene, C₁-C₆ alkylene, or C₁-C₄ alkylene), or an unsubstituted heteroalkylene (e.g., 2 to 8 membered heteroalkylene, 2 to 6 membered heteroalkylene, or 2 to 4 membered heteroalkylene); L^{6B} is an unsubstituted cycloalkylene (e.g., C₃-C₈ cycloalkylene, C₃-C₆ cycloalkylene, or C₅-C₆ cycloalkylene), unsubstituted heterocycloalkylene (e.g., 3 to 8 membered heterocycloalkylene, 3 to 6 membered heterocycloalkylene, or 5 to 6 membered heterocycloalkylene, unsubstituted arylene (e.g., C₆-C₁₀ arylene, C₁₀ arylene, or phenylene), or unsubstituted heteroarylene (e.g., 5 to 10 membered heteroarylene, 5 to 9 membered heteroarylene, or 5 to 6 membered heteroarylene); and L^{6C} is unsubstituted alkylene (e.g., C₁-C₈ alkylene, C₁-C₆ alkylene, or C₁-C₄ alkylene), or unsubstituted heteroalkylene (e.g., 2 to 8 membered heteroalkylene, 2 to 6 membered heteroalkylene, or 2 to 4 membered heteroalkylene).

In embodiments, L^{6A} is R^{18A}-substituted or unsubstituted alkylene (e.g., C₁-C₈ alkylene, C₁-C₆ alkylene, or C₁-C₄ alkylene); L^{6B} is R^{18B}-substituted or unsubstituted arylene (e.g., C₆-C₁₀ arylene, C₁₀ arylene, or phenylene); and L^{6C} is R^{18C}-substituted or unsubstituted heteroalkylene (e.g., 2 to 8 membered heteroalkylene, 2 to 6 membered heteroalkylene, or 2 to 4 membered heteroalkylene). In embodiments, L⁶ is

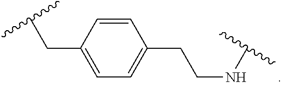 [SMILES: *Cc1ccc(CCN*)cc1]

In embodiments, L^{6A}-L^{6B}-L^{6C} is

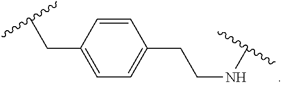 [SMILES: *Cc1ccc(CCN*)cc1]

In embodiments, R⁶ is hydrogen, —OH, —SH, —NH₂, —C(O)OH, —C(O)NH₂, R²⁰-substituted or unsubstituted alkyl (e.g., C₁-C₈ alkyl, C₁-C₆ alkyl, or C₁-C₄ alkyl), R²⁰-substituted or unsubstituted heteroalkyl (e.g., 2 to 8 membered heteroalkyl, 2 to 6 membered heteroalkyl, or 2 to 4 membered heteroalkyl), R²⁰-substituted or unsubstituted cycloalkyl (e.g., C₃-C₈ cycloalkyl, C₃-C₆ cycloalkyl, or C₅-C₆ cycloalkyl), R²⁰-substituted or unsubstituted heterocycloalkyl (e.g., 3 to 8 membered heterocycloalkyl, 3 to 6 membered heterocycloalkyl, or 5 to 6 membered heterocycloalkyl), RN-substituted or unsubstituted aryl (e.g., C₆-C₁₀ aryl, C₁₀ aryl, or phenyl), RN-substituted or unsubstituted heteroaryl (e.g., 5 to 10 membered heteroaryl, 5 to 9 membered heteroaryl, or 5 to 6 membered heteroaryl) a hydrophilic drug moiety, a targeting moiety or a detectable moiety. In embodiments, R⁶ is a hydrophilic drug moiety, a targeting moiety or a detectable moiety. In embodiments, R⁶ is a hydrophilic drug moiety. In embodiments, R⁶ is a detectable moiety. In embodiments, R⁶ is a targeting moiety.

In embodiments, R⁶ is R²⁰-substituted or unsubstituted alkyl (e.g., C₁-C₈ alkyl, C₁-C₆ alkyl, or C₁-C₄ alkyl). In embodiments, R⁶ is R²⁰-substituted alkyl (e.g., C₁-C₈ alkyl, C₁-C₆ alkyl, or C₁-C₄ alkyl). In embodiments, R⁶ is an unsubstituted alkyl (e.g., C₁-C₈ alkyl, C₁-C₆ alkyl, or C₁-C₄ alkyl).

In embodiments, R⁶ is R²⁰-substituted or unsubstituted heteroalkyl (e.g., 2 to 8 membered heteroalkyl, 2 to 6 membered heteroalkyl, or 2 to 4 membered heteroalkyl). In embodiments, R⁶ is R²⁰-substituted heteroalkyl (e.g., 2 to 8 membered heteroalkyl, 2 to 6 membered heteroalkyl, or 2 to 4 membered heteroalkyl). In embodiments, R⁶ is an unsubstituted heteroalkyl (e.g., 2 to 8 membered heteroalkyl, 2 to 6 membered heteroalkyl, or 2 to 4 membered heteroalkyl).

In embodiments, R⁶ is R²⁰-substituted or unsubstituted cycloalkyl (e.g., C₃-C₈ cycloalkyl, C₃-C₆ cycloalkyl, or C₅-C₆ cycloalkyl). In embodiments, R⁶ is R²⁰-substituted cycloalkyl (e.g., C₃-C₈ cycloalkyl, C₃-C₆ cycloalkyl, or C₅-C₆ cycloalkyl). In embodiments, R⁶ is an unsubstituted cycloalkyl (e.g., C₃-C₈ cycloalkyl, C₃-C₆ cycloalkyl, or C₅-C₆ cycloalkyl).

In embodiments, R⁶ is R²⁰-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁶ is R²⁰-substituted heterocycloalkyl (e.g., 3 to 8 membered heterocycloalkyl, 3 to 6 membered heterocycloalkyl, or 5 to 6 membered heterocycloalkyl). In embodiments, R⁶ is an unsubstituted heterocycloalkyl (e.g., 3 to 8 membered heterocycloalkyl, 3 to 6 membered heterocycloalkyl, or 5 to 6 membered heterocycloalkyl).

In embodiments, R⁶ is R²⁰-substituted or unsubstituted aryl (e.g., C₆-C₁₀ aryl, C₁₀ aryl, or phenyl). In embodiments, R⁶ is R²⁰-substituted aryl (e.g., C₆-C₁₀ aryl, C₁₀ aryl, or phenyl). In embodiments, R⁶ is an unsubstituted aryl (e.g., C₆-C₁₀ aryl, C₁₀ aryl, or phenyl).

In embodiments, R⁶ is R²⁰-substituted or unsubstituted heteroaryl (e.g., 5 to 10 membered heteroaryl, 5 to 9 membered heteroaryl, or 5 to 6 membered heteroaryl). In embodiments, R⁶ is R²⁰-substituted heteroaryl (e.g., 5 to 10 membered heteroaryl, 5 to 9 membered heteroaryl, or 5 to 6 membered heteroaryl). In embodiments, R⁶ is an unsubstituted heteroaryl (e.g., 5 to 10 membered heteroaryl, 5 to 9 membered heteroaryl, or 5 to 6 membered heteroaryl).

R²⁰ is independently oxo,

halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, R²¹-substituted or unsubstituted alkyl (e.g., C₁-C₈ alkyl, C₁-C₆ alkyl, or C₁-C₄ alkyl), R²¹-substituted or unsubstituted heteroalkyl (e.g., 2 to 8 membered heteroalkyl, 2 to 6 membered heteroalkyl, or 2 to 4 membered heteroalkyl), R²¹-substituted or unsubstituted cycloalkyl (e.g., C₃-C₈ cycloalkyl, C₃-C₆ cycloalkyl, or C₅-C₆ cycloalkyl), R²¹-substituted or unsubstituted heterocycloalkyl (e.g., 3 to 8 membered heterocycloalkyl, 3 to 6 membered heterocycloalkyl, or 5 to 6 membered heterocycloalkyl), R²¹-substituted or unsubstituted aryl (e.g., C₆-C₁₀ aryl, C₁₀ aryl, or phenyl), or R²¹-substituted or unsubstituted heteroaryl (e.g., 5 to 10 membered heteroaryl, 5 to 9 membered heteroaryl, or 5 to 6 membered heteroaryl).

R⁸, R¹⁰, R¹², R¹³, R¹⁴, R¹⁵, R¹⁶, R¹⁷, R^{18A}, R^{18B}, R^{18C}, R¹⁹, R²¹, R^{22A}, R^{23A}, R^{22B}, R^{23B}, R^{22C}, R^{23C}, R^{22D}, R^{23D}, R²⁶, R²⁸, R³⁰, and R³² are independently oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, L^{1A}, L^{2A}, L^{1B}, L^{2B}, L^{1C}, L^{2C}, L^{1D}, L^{2D} are independently a bond or a substituted or unsubstituted alkylene. In embodiments, L^{1A}, L^{2A}, L^{1B}, L^{2B}, L^{1C}, L^{2C}, L^{1D}, L² are independently a bond or a substituted or unsubstituted alkenylene. In embodiments, L^{1A}, L^{2A}, L^{1B}, L^{2B}, L^{1C}, L^{2C}, L^{1D}, L^{2D} are independently a substituted or unsubstituted alkenylene. In embodiments, L^{1A}, L^{2A}, L^{1B}, L^{2B}, L^{1C}, L^{2C}, L^{1D}, L^{2D} are independently unsubstituted alkenylene.

In embodiments, L^{1A}, L^{2A}, L^{1B}, L^{2B}, L^{1C}, L^{2C}, L^{1D}, L^{2D} are independently a bond or a substituted or unsubstituted alkylene (e.g., C₁-C₂₀ alkylene, C₁-C₁₈ alkylene, C₁-C₁₂ alkylene, C₁-C₁₀ alkylene, C₁-C₈ alkylene, C₁-C₆ alkylene, C₁-C₄ alkylene, or C₁-C₂ alkylene). In embodiments, L^{1A}, L^{2A}, L^{1B}, L^{2B}, L^{1C}, L^{1D}, L^{2D} are independently a bond or a substituted or unsubstituted alkenylene (e.g., C₂-C₂₀ alkenylene, C₂-C₁₈ alkenylene, C₂-C₁₂ alkenylene, C₂-C₁₀ alkenylene, C₂-C₈ alkenylene, C₂-C₆ alkenylene, C₂-C₄ alkenylene, or C₂ alkenylene). In embodiments, L^{1A}, L^{2A}, L^{1B}, L^{2B}, L^{1C}, L^{2C}, L^{1D}, L^{2D} are independently a substituted or unsubstituted alkenylene (e.g., C₂-C₂₀ alkenylene, C₂-C₁₈ alkenylene, C₂-C₁₂ alkenylene, C₂-C₁₀ alkenylene, C₂-C₈ alkenylene, C₂-C₆ alkenylene, C₂-C₄ alkenylene, or C₂ alkenylene). In embodiments, L^{1A}, L^{2A}, L^{1B}, L^{2B}, L^{1C}, L^{2C}, L^{1D}, L^{2D} are independently an unsubstituted alkenylene (e.g., C₂-C₂₀ alkenylene, C₂-C₁₈ alkenylene, C₂-C₁₂ alkenylene, C₂-C₁₀ alkenylene, C₂-C₈ alkenylene, C₂-C₆ alkenylene, C₂-C₄ alkenylene, or C₂ alkenylene).

In embodiments, z1 is an integer from 0 to 100. In embodiments, z1 is an integer from 0 to 50. In embodiments, z1 is an integer from 0 to 30. In embodiments, z1 is an integer from 0 to 20. In embodiments, z1 is an integer from 0 to 10. In embodiments, z1 is an integer from 0 to 5. In embodiments, z1 is an integer from 5 to 100. In embodiments, z1 is an integer from 10 to 100. In embodiments, z1 is an integer from 20 to 100. In embodiments, z1 is an integer from 5 to 50. In embodiments, z1 is an integer from 10 to 50. In embodiments, z1 is an integer from 20 to 50. In embodiments, z1 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In embodiments, z3 is an integer from 0 to 100. In embodiments, z3 is an integer from 0 to 50. In embodiments, z3 is an integer from 0 to 30. In embodiments, z3 is an integer from 0 to 20. In embodiments, z3 is an integer from 0 to 10. In embodiments, z3 is an integer from 0 to 5. In embodiments, z3 is an integer from 5 to 100. In embodiments, z3 is an integer from 10 to 100. In embodiments, z3 is an integer from 20 to 100. In embodiments, z3 is an integer from 5 to 50. In embodiments, z3 is an integer from 10 to 50. In embodiments, z3 is an integer from 20 to 50. In embodiments, z3 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In embodiments, z5 is an integer from 0 to 100. In embodiments, z5 is an integer from 0 to 50. In embodiments, z5 is an integer from 0 to 30. In embodiments, z5 is an integer from 0 to 20. In embodiments, z5 is an integer from 0 to 10. In embodiments, z5 is an integer from 0 to 5. In embodiments, z5 is an integer from 5 to 100. In embodiments, z5 is an integer from 10 to 100. In embodiments, z5 is an integer from 20 to 100. In embodiments, z5 is an integer from 5 to 50. In embodiments, z5 is an integer from 10 to 50. In embodiments, z5 is an integer from 20 to 50. In embodiments, z5 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In embodiments, z7 is an integer from 0 to 100. In embodiments, z7 is an integer from 0 to 50. In embodiments, z7 is an integer from 0 to 30. In embodiments, z7 is an integer from 0 to 20. In embodiments, z7 is an integer from 0 to 10. In embodiments, z7 is an integer from 0 to 5. In embodiments, z7 is an integer from 5 to 100. In embodiments, z7 is an integer from 10 to 100. In embodiments, z7 is an integer from 20 to 100. In embodiments, z7 is an integer from 5 to 50. In embodiments, z7 is an integer from 10 to 50. In embodiments, z7 is an integer from 20 to 50. In embodiments, z7 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In embodiments, z2 is an integer from 1 to 100. In embodiments, z2 is an integer from 1 to 50. In embodiments, z2 is an integer from 1 to 30. In embodiments, z2 is an integer from 1 to 20. In embodiments, z2 is an integer from 1 to 10. In embodiments, z2 is an integer from 1 to 5. In embodiments, z2 is an integer from 5 to 100. In embodiments, z2 is an integer from 10 to 100. In embodiments, z2 is an integer from 20 to 100. In embodiments, z2 is an integer from 5 to 50. In embodiments, z2 is an integer from 10 to 50. In embodiments, z2 is an integer from 20 to 50. In embodiments, z2 is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In embodiments, z4 is an integer from 1 to 100. In embodiments, z4 is an integer from 1 to 50. In embodiments, z4 is an integer from 1 to 30. In embodiments, z4 is an integer from 1 to 20. In embodiments, z4 is an integer from 1 to 10. In embodiments, z4 is an integer from 1 to 5. In embodiments, z4 is an integer from 5 to 100. In embodiments, z4 is an integer from 10 to 100. In embodiments, z4 is an integer from 20 to 100. In embodiments, z4 is an integer from 5 to 50. In embodiments, z4 is an integer from 10 to 50. In embodiments, z4 is an integer from 20 to 50. In embodiments, z4 is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. In embodiments, z4 is 1.

In embodiments, z6 is an integer from 1 to 100. In embodiments, z6 is an integer from 1 to 50. In embodiments, z6 is an integer from 1 to 30. In embodiments, z6 is an integer from 1 to 20. In embodiments, z6 is an integer from 1 to 10. In embodiments, z6 is an integer from 1 to 5. In embodiments, z6 is an integer from 5 to 100. In embodiments, z6 is an integer from 10 to 100. In embodiments, z6 is an integer from 20 to 100. In embodiments, z6 is an integer from 5 to 50. In embodiments, z6 is an integer from 10 to 50. In embodiments, z6 is an integer from 20 to 50. In embodiments, z6 is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In embodiments, z8 is an integer from 1 to 100. In embodiments, z8 is an integer from 1 to 50. In embodiments, z8 is an integer from 1 to 30. In embodiments, z8 is an integer from 1 to 20. In embodiments, z8 is an integer from 1 to 10. In embodiments, z8 is an integer from 1 to 5. In embodiments, z8 is an integer from 5 to 100. In embodiments, z8 is an integer from 10 to 100. In embodiments, z8 is an integer from 20 to 100. In embodiments, z8 is an integer from 5 to 50. In embodiments, z8 is an integer from 10 to 50. In embodiments, z8 is an integer from 20 to 50. In embodiments, z8 is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. In embodiments, z8 is 1.

In embodiments, z1 and z3 are 0. In embodiments, z5 and z7 are 0. In embodiments, z4 and z8 are 1. In embodiments, z2 is an integer from 10-35 and z6 is 2 or 4. In embodiments, z2 is an integer from 15-35 and z6 is 2 or 4. In embodiments, z2 is an integer from 10-30 and z6 is 2 or 4. In embodiments, z2 is an integer from 15-25 and z6 is 2 or 4. In embodiments, z2 is an integer from 10-35 and z6 is 2. In embodiments, z2 is an integer from 15-35 and z6 is 2. In embodiments, z2 is an integer from 10-30 and z6 is 2. In embodiments, z2 is an integer from 15-25 and z6 is 2. In embodiments, z2 is an integer from 10-35 and z6 is 4. In embodiments, z2 is an integer from 15-35 and z6 is 4. In embodiments, z2 is an integer from 10-30 and z6 is 4. In embodiments, z2 is an integer from 15-25 and z6 is 4. In embodiments, z2 is an integer from 10-35 and z6 is 3. In embodiments, z2 is an integer from 15-35 and z6 is 3. In embodiments, z2 is an integer from 10-30 and z6 is 3. In embodiments, z2 is an integer from 15-25 and z6 is 3. In embodiments, z2 is an integer from 10-35, z5 is an integer from 1 to 6, z6 is 3 or 4. In embodiments, z1 and z3 are 0 and z5 is not 0.

In embodiments, the polymer includes a single (i.e. a quantity of one) drug moiety. In embodiments, the copolymer includes a single drug moiety. In embodiments, the polymer includes a single (i.e. a quantity of one) targeting moiety. In embodiments, the copolymer includes a single (i.e. a quantity of one) targeting moiety. In embodiments, the polymer includes a single (i.e. a quantity of one) detectable moiety. In embodiments, the copolymer includes a single (i.e. a quantity of one) detectable moiety.

In embodiments, the polymer includes a single (i.e. one type) drug moiety. In embodiments, the copolymer includes a single drug moiety. In embodiments, the polymer includes a single (i.e. one type) targeting moiety. In embodiments, the copolymer includes a single (i.e. one type) targeting moiety. In embodiments, the polymer includes a single (i.e. one type) detectable moiety. In embodiments, the copolymer includes a single (i.e. one type) detectable moiety.

In an aspect is provided a polymeric micelle including a plurality of the block copolymer as described herein, the polymeric micelle including a hydrophobic core including the first block of hydrophobic polymerized monomers and a hydrophilic shell including the second block of hydrophilic polymerized monomers.

In embodiments, the inflammatory protease cleavable amino acid sequence is cleaved between the G (i.e. glycine) and L (i.e. leucine) amino acid residues of the inflammatory protease cleavable amino acid sequence. In embodiments, the inflammatory protease cleavable amino acid sequence comprises L-amino acid residues. In embodiments, the inflammatory protease cleavable amino acid sequence is GPLGLAGGWGERDGS (SEQ ID NO:1). In embodiments, the inflammatory protease cleavable amino acid sequence is PLGLAG (SEQ ID NO:2). In embodiments, the inflammatory protease cleavable amino acid sequence is LAGGWGERDGS (SEQ ID NO:3). In embodiments, the inflammatory protease cleavable amino acid sequence is GPLGLAGGERDG (SEQ ID NO:4). In embodiments, the inflammatory protease cleavable amino acid sequence contains GPLGLAGGWGERDGS (SEQ ID NO:1). In embodiments, the inflammatory protease cleavable amino acid sequence contains PLGLAG (SEQ ID NO:2). In embodiments, the inflammatory protease cleavable amino acid sequence contains LAGGWGERDGS (SEQ ID NO:3). In embodiments, the inflammatory protease cleavable amino acid sequence contains GPLGLAGGERDG (SEQ ID NO:4).

In embodiments, the cardiovascular inflammatory protease cleavable amino acid sequence is cleaved between the G (i.e. glycine) and L (i.e. leucine) amino acid residues of the cardiovascular inflammatory protease cleavable amino acid sequence. In embodiments, the cardiovascular inflammatory protease cleavable amino acid sequence comprises L-amino acid residues. In embodiments, the cardiovascular inflammatory protease cleavable amino acid sequence is GPLGLAGGWGERDGS (SEQ ID NO:1). In embodiments, the cardiovascular inflammatory protease cleavable amino acid sequence is PLGLAG (SEQ ID NO:2). In embodiments, the cardiovascular inflammatory protease cleavable amino acid sequence is LAGGWGERDGS (SEQ ID NO:3). In embodiments, the cardiovascular inflammatory protease cleavable amino acid sequence is GPLGLAGGERDG (SEQ ID NO:4). In embodiments, the cardiovascular inflammatory protease cleavable amino acid sequence contains GPLGLAGGWGERDGS (SEQ ID NO:1). In embodiments, the cardiovascular inflammatory protease cleavable amino acid sequence contains PLGLAG (SEQ ID NO:2). In embodiments, the cardiovascular inflammatory protease cleavable amino acid sequence contains LAGGWGERDGS (SEQ ID NO:3). In embodiments, the cardiovascular inflammatory protease cleavable amino acid sequence contains GPLGLAGGERDG (SEQ ID NO:4).

In embodiments, the myocardial inflammatory protease cleavable amino acid sequence is cleaved between the G (i.e. glycine) and L (i.e. leucine) amino acid residues of the myocardial inflammatory protease cleavable amino acid sequence. In embodiments, the myocardial inflammatory protease cleavable amino acid sequence comprises L-amino acid residues. In embodiments, the myocardial inflammatory protease cleavable amino acid sequence is GPLGLAGGWGERDGS (SEQ ID NO:1). In embodiments, the myocardial inflammatory protease cleavable amino acid sequence is PLGLAG (SEQ ID NO:2). In embodiments, the myocardial inflammatory protease cleavable amino acid sequence is LAGGWGERDGS (SEQ ID NO:3). In embodiments, the myocardial inflammatory protease cleavable amino acid sequence is GPLGLAGGERDG (SEQ ID NO:4). In embodiments, the myocardial inflammatory protease cleavable amino acid sequence contains GPLGLAGGWGERDGS (SEQ ID NO:1). In embodiments, the myocardial inflammatory protease cleavable amino acid sequence contains PLGLAG (SEQ ID NO:2). In embodiments, the myocardial inflammatory protease cleavable amino acid sequence contains LAGGWGERDGS (SEQ ID NO:3). In embodiments, the myocardial inflammatory protease cleavable amino acid sequence contains GPLGLAGGERDG (SEQ ID NO:4).

In embodiments, the MMP cleavable amino acid sequence is an MMP-2 or MMP-9 cleavable amino acid sequence. In embodiments, the MMP cleavable amino acid sequence is analogous sequence known to be a substrate of a matrix metalloproteinase (MMP). In embodiments, the MMP cleavable amino acid sequence is an MMP-9 cleavable amino acid sequence. In embodiments, the MMP cleavable amino acid sequence is an MMP-2 cleavable amino acid sequence. In embodiments, the MMP cleavable amino acid sequence is an MMP-9 cleavable amino acid sequence. In embodiments, the MMP cleavable amino acid sequence is cleaved between the G (i.e. glycine) and L (i.e. leucine) amino acid residues of the MMP cleavable amino acid sequence. In embodiments, the MMP-2 cleavable amino acid sequence is cleaved between the G (i.e. glycine) and L (i.e. leucine) amino acid residues of the MMP-2 cleavable amino acid sequence. In embodiments, the MMP-9 cleavable amino acid sequence is cleaved between the G (i.e. glycine) and L (i.e. leucine) amino acid residues of the MMP-9 cleavable amino acid sequence.

In embodiments, the MMP cleavable amino acid sequence comprises L-amino acid residues. In embodiments, the MMP-2 cleavable amino acid sequence comprises L-amino acid residues. In embodiments, the MMP-2 cleavable amino acid sequence comprises L-amino acid residues. In embodiments, the MMP cleavable amino acid sequence is GPLGLAGGWGERDGS (SEQ ID NO:1). In embodiments, the MMP cleavable amino acid sequence is PLGLAG (SEQ ID NO:2). In embodiments, the MMP cleavable amino acid sequence is LAGGWGERDGS (SEQ ID NO:3). In embodiments, the MMP cleavable amino acid sequence is GPLGLAGGERDG (SEQ ID NO:4). In embodiments, the MMP cleavable amino acid sequence contains GPLGLAGGWGERDGS (SEQ ID NO:1). In embodiments, the MMP cleavable amino acid sequence contains PLGLAG (SEQ ID NO:2). In embodiments, the MMP cleavable amino acid sequence contains LAGGWGERDGS (SEQ ID NO:3). In embodiments, the MMP cleavable amino acid sequence contains GPLGLAGGERDG (SEQ ID NO:4).

In embodiments, the MMP-2 cleavable amino acid sequence is GPLGLAGGWGERDGS (SEQ ID NO:1). In embodiments, the MMP-2 cleavable amino acid sequence is PLGLAG (SEQ ID NO:2). In embodiments, the MMP-2 cleavable amino acid sequence is LAGGWGERDGS (SEQ ID NO:3). In embodiments, the MMP-2 cleavable amino acid sequence is GPLGLAGGERDG (SEQ ID NO:4). In embodiments, the MMP-9 cleavable amino acid sequence is GPLGLAGGWGERDGS (SEQ ID NO:1). In embodiments, the MMP-9 cleavable amino acid sequence is PLGLAG (SEQ ID NO:2). In embodiments, the MMP-9 cleavable amino acid sequence is LAGGWGERDGS (SEQ ID NO:3). In embodiments, the MMP-9 cleavable amino acid sequence is GPLGLAGGERDG (SEQ ID NO:4).

In embodiments, the MMP-2 cleavable amino acid sequence contains GPLGLAGGWGERDGS (SEQ ID NO:1). In embodiments, the MMP-2 cleavable amino acid sequence contains PLGLAG (SEQ ID NO:2). In embodiments, the MMP-2 cleavable amino acid sequence contains LAGGWGERDGS (SEQ ID NO:3). In embodiments, the MMP-2 cleavable amino acid sequence contains GPLGLAGGERDG (SEQ ID NO:4). In embodiments, the MMP-9 cleavable amino acid sequence contains GPLGLAGGWGERDGS (SEQ ID NO:1). In embodiments, the MMP-9 cleavable amino acid sequence contains PLGLAG (SEQ ID NO:2). In embodiments, the MMP-9 cleavable amino acid sequence contains LAGGWGERDGS (SEQ ID NO:3). In embodiments, the MMP-9 cleavable amino acid sequence contains GPLGLAGGERDG (SEQ ID NO:4).

In embodiments, the targeting moiety is an antibody, peptide sequence (e.g. peptide sequence capable of recognizing receptors such as an integrin), nucleic acid, or a small molecule (e.g., small molecule capable of binding a folic acid receptor). In embodiments, the targeting moiety is targets biochemical markers (e.g., proteins) of myocardial necrosis (e.g., cardiac troponins). In embodiments, the targeting moiety is targets biochemical markers (e.g., proteins) of myocardial infarction.

In embodiments, the drug moiety is an antiplatelet agent (e.g., aspirin, clopidogrel, ticagrelor), antithrombotic agent (heparin, enoxaparin), beta-adrenergic blocker (e.g., metoprolol, atenolol, esmolol), thrombolytic (e.g., alteplase, tenecteplase), or analgesic (e.g., morphine). In embodiments, the drug moiety is an antiplatelet agent (e.g., aspirin, clopidogrel, ticagrelor). In embodiments, the drug moiety is an antithrombotic agent (heparin, enoxaparin). In embodiments, the drug moiety is a beta-adrenergic blocker (e.g., metoprolol, atenolol, esmolol). In embodiments, the drug moiety is a thrombolytic (e.g., alteplase, tenecteplase). In embodiments, the drug moiety is an analgesic (e.g., morphine). In embodiments, the drug moiety is nitroglycerine. In embodiments, the drug moiety is hydrophobic. In embodiments, the drug moiety is hydrophilic. In embodiments, the drug moiety is not hydrophobic.

In embodiments, the drug moiety is a peptide, small molecule, or nucleic acid. In embodiments, the drug moiety is an MMP inhibitor (e.g., inhibits MMP activity). In embodiments, the drug moiety is an anti-inflammatory. In embodiments, the drug moiety promotes myocardial salvage, increases cardiomyocyte survival, increases neovascularization, modulates (e.g., reduces relative to the absence of the drug) the inflammatory response, modulates (e.g., reduces relative to the absence of the drug) cardiomyocyte metabolism, reduces infarct size, reduces fibrosis (e.g., cardiac fibrosis), reduces or inhibits negative LV remodeling, reduces LV volume (e.g. diastolic and/or systolic), increases infarct wall thickness, inhibits MMP activity, or prevents extracellular matrix degradation. In embodiments, the drug moiety is an inhibitor of proteolytic enzymes (e.g., TIMP1, TIMP2, TIMP3, TIMP4, calpastatin, serpin, lipocalin).

### III. Pharmaceutical Compositions

In another aspect is an aqueous pharmaceutical composition including the block copolymer as described herein and a pharmaceutically acceptable excipient. In embodiments, the pharmaceutical composition is a parental dosage form. In embodiments, the pharmaceutical composition is an intravenous dosage form.

In another aspect is an aqueous pharmaceutical composition including the polymeric micelle as described herein and a pharmaceutically acceptable excipient. In embodiments, the pharmaceutical composition is a parental dosage form. In embodiments, the pharmaceutical composition is an intravenous dosage form. In embodiments, the pharmaceutical composition is an intramyocardial dosage form.

In embodiments, the pharmaceutical composition is isotonic. In embodiments, the pharmaceutical composition is isotonic to human blood. The pharmaceutical composition may have a pH from about 3.5 to about 6.2. In embodiments, the pharmaceutical composition is isotonic. In embodiments, the pharmaceutical composition is isotonic and has a pH from about 3.5 to about 6.2. In embodiments, the pharmaceutical composition is isotonic and has a pH from about 4.0 to about 6.2. In embodiments, the pharmaceutical composition is isotonic and has a pH from about 4.5 to about 6.2. In embodiments, the pharmaceutical composition is isotonic and has a pH from about 5.0 to about 6.2. In embodiments, the pharmaceutical composition is isotonic and has a pH from about 5.5 to about 6.2.

In embodiments, the pharmaceutical composition has a pH from about 3.5 to about 6.2. In embodiments, the pharmaceutical composition has a pH from about 4.0 to about 6.2. In embodiments, the pharmaceutical composition has a pH from about 4.5 to about 6.2. In embodiments, the pharmaceutical composition has a pH from about 5.0 to about 6.2. In embodiments, the pharmaceutical composition has a pH from about 5.5 to about 6.2.

In embodiments, the pharmaceutical composition has a pH from about 3.5 to about 8.2. In embodiments, the pharmaceutical composition has a pH from about 4.0 to about 8.2. In embodiments, the pharmaceutical composition has a pH from about 4.5 to about 8.2. In embodiments, the pharmaceutical composition has a pH from about 5.0 to about 8.2. In embodiments, the pharmaceutical composition has a pH from about 5.5 to about 8.2. In embodiments, the pharmaceutical composition has a pH from about 6.0 to about 8.2. In embodiments, the pharmaceutical composition has a pH from about 6.5 to about 8.2. In embodiments, the pharmaceutical composition has a pH from about 7.0 to about 8.2. In embodiments, the pharmaceutical composition has a pH from about 7.5 to about 8.2. In embodiments, the pharmaceutical composition has a pH from about 7.0 to about 8.0. In embodiments, the pharmaceutical composition has a pH about 7.8.

### IV. Methods of Use

In an aspect is provided a method of treating a myocardial infarction in a subject in need thereof, the method including administering to the subject a therapeutically effective amount of the block copolymer, the polymeric micelle, or the pharmaceutical composition as described herein.

In an aspect is provided a method of treating a myocardial infarction in a subject in need thereof, the method including administering to the subject a therapeutically effective amount of the block copolymer as described herein or the pharmaceutical composition as described herein.

In embodiments, the method improves cardiac function (e.g., promotes myocardial salvage), increases cardiomyocyte survival, increases neovascularization, modulates (e.g., reduces relative to the absence of the block copolymer or pharmaceutical composition) the inflammatory response, modulates (e.g., reduces relative to the absence of the block copolymer or pharmaceutical composition) cardiomyocyte metabolism, reduces infarct size, reduces fibrosis (e.g., cardiac fibrosis), reduces or inhibits negative LV remodeling, reduces LV volume (e.g. diastolic and/or systolic), increases infarct wall thickness, inhibits MMP activity, or prevents extracellular matrix degradation following administration.

In embodiments, the method improves cardiac function (e.g., promotes myocardial salvage). In embodiments, the method increases cardiomyocyte survival. In embodiments, the method increases neovascularization. In embodiments, the method modulates (e.g., reduces relative to the absence of the block copolymer or pharmaceutical composition) the inflammatory response. In embodiments, the method modulates (e.g., reduces relative to the absence of the block copolymer or pharmaceutical composition) cardiomyocyte metabolism. In embodiments, the method reduces infarct size. In embodiments, the method reduces fibrosis (e.g., cardiac fibrosis). In embodiments, the method reduces or inhibits negative LV remodeling. In embodiments, the method reduces LV volume (e.g. diastolic and/or systolic). In embodiments, the method increases infarct wall thickness. In embodiments, the method inhibits MMP activity. In embodiments, the method prevents extracellular matrix degradation.

In an aspect is provided a method of treating a myocardial infarction in a subject in need thereof, the method including administering to the subject a therapeutically effective amount of the polymeric micelle as described herein or the pharmaceutical composition as described herein. In embodiments, the method improves cardiac function following administration.

In an aspect is provided a method of treating a disease (e.g., myocardial infarction, cardiomyopathy, heart failure) in a subject in need thereof, the method including administering to the subject a therapeutically effective amount of the polymeric micelle as described herein or the pharmaceutical composition as described herein. In embodiments, the method improves cardiac function following administration.

In another aspect, provided herein is a method of treating heart failure in a subject in need thereof, the method including administering to the subject a therapeutically effective amount of the block copolymer, the polymeric micelle, or the pharmaceutical composition as described herein.

In embodiments, the method inhibits negative left ventricular (LV) remodeling relative to negative LV remodeling in the absence of the compositions (e.g., block copolymers or polymeric micelles) described herein. In embodiments, the method reduces negative left ventricular (LV) remodeling relative to negative LV remodeling in the absence of the compositions (e.g., block copolymers or polymeric micelles) described herein. In embodiments, the method slows negative left ventricular (LV) remodeling relative to negative LV remodeling in the absence of the compositions (e.g., block copolymers or polymeric micelles) described herein.

In embodiments, the block copolymer or the pharmaceutical composition is administered intravenously. In embodiments, the block copolymer or the pharmaceutical composition is administered via intramyocardial delivery. In embodiments, the block copolymer or the pharmaceutical composition is administered via intracoronary delivery. In embodiments, the block copolymer or the pharmaceutical composition is administered via intracoronary infusion, retrograde injection, or injection into adventitial space. In embodiments, the block copolymer or the pharmaceutical composition is administered following angioplasty or following stent placement.

In embodiments, the polymeric micelle or the pharmaceutical composition is administered intravenously. In embodiments, the polymeric micelle or the pharmaceutical composition is administered via intramyocardial delivery. In embodiments, the polymeric micelle or the pharmaceutical composition is administered via intracoronary delivery.

In another aspect is provided a method of forming a polymeric aggregate, the method including i) contacting the block copolymer as described herein with an inflammatory protease; and ii) allowing the inflammatory protease to cleave the inflammatory protease cleavable amino acid sequence, thereby forming a polymeric aggregate.

In another aspect is provided a method of forming a polymeric aggregate, the method including i) contacting the polymeric micelle as described herein with an inflammatory protease;

and ii) allowing the inflammatory protease to cleave the inflammatory protease cleavable amino acid sequence, thereby forming a polymeric aggregate. In embodiments, the polymeric aggregate forms immediately following contact with an inflammatory protease enzyme. In embodiments, the polymeric aggregate forms within 1 minute following contact with an inflammatory protease enzyme. In embodiments, the polymeric aggregate forms within 5 minutes following contact with an inflammatory protease enzyme. In embodiments, the polymeric aggregate forms within 10 minutes following contact with an inflammatory protease enzyme. In embodiments, the polymeric aggregate forms within 30 minutes following contact with an inflammatory protease enzyme. In embodiments, the polymeric aggregate forms within 1 hour following contact with an inflammatory protease enzyme. In embodiments, the polymeric aggregate forms within 2 hours following contact with an inflammatory protease enzyme. In embodiments, the polymeric aggregate forms within 1 day following contact with an inflammatory protease enzyme. In embodiments, the polymeric aggregate forms within 2 days following contact with an inflammatory protease enzyme. In embodiments, the polymeric aggregate forms within 3 days following contact with an inflammatory protease enzyme. In embodiments, the polymeric aggregate forms within 7 days following contact with an inflammatory protease enzyme. In embodiments, the polymeric aggregate forms within 8 days following contact with an inflammatory protease enzyme. In embodiments, the polymeric aggregate forms within 9 days following contact with an inflammatory protease enzyme. In embodiments, the polymeric aggregate forms within 10 days following contact with an inflammatory protease enzyme. In embodiments, the polymeric aggregate forms within 2 weeks following contact with an inflammatory protease enzyme. In embodiments, the inflammatory protease is a cardiovascular inflammatory protease. In embodiments, the inflammatory protease is a myocardial inflammatory protease.

In another aspect is provided a method of forming a polymeric aggregate, the method including i) contacting the block copolymer as described herein with an MMP-2 or MMP-9; and ii) allowing the MMP-2 or MMP-9 to cleave the MMP-2 or MMP-9 cleavable amino acid sequence, respectively, thereby forming a polymeric aggregate. In embodiments, forming is when two or more cleaved block copolymers contact and create an aggregate.

In another aspect is provided a method of forming a polymeric aggregate, the method including i) contacting the polymeric micelle as described herein with an MMP-2 or MMP-9; and ii) allowing the MMP-2 or MMP-9 to cleave the MMP-2 or MMP-9 cleavable amino acid sequence, respectively, thereby forming a polymeric aggregate. In embodiments, the polymeric aggregate forms immediately following contact with an MMP-2 or MMP-9 enzyme. In embodiments, the polymeric aggregate forms within 1 minute following contact with an MMP-2 or MMP-9 enzyme. In embodiments, the polymeric aggregate forms within 5 minutes following contact with an MMP-2 or MMP-9 enzyme. In embodiments, the polymeric aggregate forms within 10 minutes following contact with an MMP-2 or MMP-9 enzyme. In embodiments, the polymeric aggregate forms within 30 minutes following contact with an MMP-2 or MMP-9 enzyme. In embodiments, the polymeric aggregate forms within 1 hour following contact with an MMP-2 or MMP-9 enzyme. In embodiments, the polymeric aggregate forms within 2 hours following contact with an MMP-2 or MMP-9 enzyme. In embodiments, the polymeric aggregate forms within 1 day following contact with an MMP-2 or MMP-9 enzyme. In embodiments, the polymeric aggregate forms within 2 days following contact with an MMP-2 or MMP-9 enzyme. In embodiments, the polymeric aggregate forms within 3 days following contact with an MMP-2 or MMP-9 enzyme. In embodiments, the polymeric aggregate forms within 7 days following contact with an MMP-2 or MMP-9 enzyme. In embodiments, the polymeric aggregate forms within 8 days following contact with an MMP-2 or MMP-9 enzyme. In embodiments, the polymeric aggregate forms within 9 days following contact with an MMP-2 or MMP-9 enzyme. In embodiments, the polymeric aggregate forms within 10 days following contact with an MMP-2 or MMP-9 enzyme. In embodiments, the polymeric aggregate forms within 2 weeks following contact with an MMP-2 or MMP-9 enzyme.

In embodiments, the polymeric micelles (e.g. nanoparticles) provided herein are delivered to a patient's heart (e.g., via intravenous, intracoronary, or intramyocardial delivery) within the first hour post-MI. In embodiments, the polymeric micelles (e.g. nanoparticles) provided herein are delivered to a patient's heart (e.g., via intravenous, intracoronary, or intramyocardial delivery) within the first two hours post-MI. In embodiments, the polymeric micelles (e.g. nanoparticles) provided herein are delivered to a patient's heart (e.g., via intravenous, intracoronary, or intramyocardial delivery) within three hours post-MI. In embodiments, the polymeric micelles (e.g. nanoparticles) provided herein are delivered to a patient's heart (e.g., via intravenous, intracoronary, or intramyocardial delivery) within four hours post-MI. In embodiments, the polymeric micelles (e.g. nanoparticles) provided herein are delivered to a patient's heart (e.g., via intravenous, intracoronary, or intramyocardial delivery) within six hours post-MI. In embodiments, the polymeric micelles (e.g. nanoparticles) provided herein are delivered to a patient's heart (e.g., via intravenous, intracoronary, or intramyocardial delivery) within eight hours post-MI. In embodiments, the polymeric micelles (e.g. nanoparticles) provided herein are delivered to a patient's heart (e.g., via intravenous, intracoronary, or intramyocardial delivery) within 12 hours post-MI. In embodiments, the polymeric micelles (e.g. nanoparticles) provided herein are delivered to a patient's heart (e.g., via intravenous, intracoronary, or intramyocardial delivery) within 18 hours post-MI. In embodiments, the polymeric micelles (e.g. nanoparticles) provided herein are delivered to a patient's heart (e.g., via intravenous, intracoronary, or intramyocardial delivery) within 24 hours post-MI. In embodiments, the polymeric micelles (e.g. nanoparticles) provided herein are delivered to a patient's heart (e.g., via intravenous, intracoronary, or intramyocardial delivery) within 48 hours post-MI. In embodiments, the polymeric micelles (e.g. nanoparticles) provided herein are delivered to a patient's heart (e.g., via intravenous, intracoronary, or intramyocardial delivery) within 72 hours post-MI. In embodiments, the polymeric micelles (e.g. nanoparticles) provided herein are delivered to a patient's heart (e.g., via intravenous, intracoronary, or intramyocardial delivery) within 96 hours post-MI.

In embodiments, the polymeric aggregate (e.g., scaffold, network, assembly) remains within the infarct for 1, 2, 3, 4, 5, 6, 7, 8, 9, 10, 11, 12, 13, 14, 15, 16, 17, 18, 19, 20, 21, 22, 23, 24, 25, 26, 27, or 28 days. In embodiments, the polymeric aggregate (e.g., scaffold, network, assembly) remaining within the infarct for up to 3 days. In embodiments, the polymeric aggregate (e.g., scaffold, network, assembly) remaining within the infarct for up to 5 days. In embodiments, the polymeric aggregate (e.g., scaffold, network, assembly) remaining within the infarct for up to 7 days. In embodiments, the polymeric aggregate (e.g., scaffold, network, assembly) remaining within the infarct for up to 14 days. In embodiments, the polymeric aggregate (e.g., scaffold, network, assembly) remaining within the infarct for up to 21 days. In embodiments, the polymeric aggregate (e.g., scaffold, network, assembly) remaining within the infarct for up to 28 days.

The polymeric micelles (e.g. nanoparticles) may possess retention times on the order of one week to months. In embodiments, the polymeric micelles (e.g. nanoparticles) are retained in the subject for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days following administration. In embodiments, the polymeric micelles (e.g. nanoparticles) are retained in the subject for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, 50, 51, or 52 weeks following administration.

In another aspect, provided herein is a method of performing cardiovascular surgery in a subject in need thereof, the method including performing a surgical procedure; and administering to the subject a therapeutically effective amount of the block copolymer, the polymeric micelle, or the pharmaceutical composition as described herein. In embodiments, the surgical procedure is an angioplasty or stent placement. In embodiments, the surgical procedure is stent placement. In embodiments, the surgical procedure is an angioplasty.

In embodiments, the polymeric aggregates do not form in the blood vessels. In embodiments, the polymeric aggregates preferentially form at or around the site of inflammation (e.g., site of infarction). In embodiments, the polymeric aggregates preferentially form at or around the heart. In embodiments, the polymeric aggregates preferentially form at or around the heart experiencing heart failure. In embodiments, the polymeric aggregates preferentially form at or around the heart following myocardial infarction.

In embodiments, the polymeric aggregates preferentially form at or around the site of inflammation (e.g., site of infarction) relative to satellite organs (e.g., kidney, liver, spleen, lungs). In embodiments, the polymeric aggregates preferentially form at or around the heart relative to satellite organs (e.g., kidney, liver, spleen, lungs). In embodiments, the polymeric aggregates preferentially form at or around the heart experiencing heart failure relative to satellite organs (e.g., kidney, liver, spleen, lungs). In embodiments, the polymeric aggregates preferentially form at or around the heart following myocardial infarction relative to satellite organs (e.g., kidney, liver, spleen, lungs).

In embodiments, the polymeric aggregates form at 1.1 fold, 1.2 fold, 1.3 fold, 1.4 fold, 1.5 fold, 1.6 fold, 1.7 fold, 1.8 fold, 1.9 fold, 2.0 fold, 5 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 500 fold, 1000 fold, or 10000 fold or around the site of inflammation (e.g., site of infarction) relative to satellite organs (e.g., kidney, liver, spleen, lungs). In embodiments, the polymeric aggregates form at 1.1 fold, 1.2 fold, 1.3 fold, 1.4 fold, 1.5 fold, 1.6 fold, 1.7 fold, 1.8 fold, 1.9 fold, 2.0 fold, 5 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 500 fold, 1000 fold, or 10000 fold or around the heart relative to satellite organs (e.g., kidney, liver, spleen, lungs). In embodiments, the polymeric aggregates form at 1.1 fold, 1.2 fold, 1.3 fold, 1.4 fold, 1.5 fold, 1.6 fold, 1.7 fold, 1.8 fold, 1.9 fold, 2.0 fold, 5 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 500 fold, 1000 fold, or 10000 fold or around the heart experiencing heart failure relative to satellite organs (e.g., kidney, liver, spleen, lungs). In embodiments, the polymeric aggregates preferentially form at or around the heart following myocardial infarction relative to satellite organs (e.g., kidney, liver, spleen, lungs). In embodiments, the polymeric aggregates form at 1.1 fold, 1.2 fold, 1.3 fold, 1.4 fold, 1.5 fold, 1.6 fold, 1.7 fold, 1.8 fold, 1.9 fold, 2.0 fold, 5 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 500 fold, 1000 fold, or 10000 fold at or around the heart following myocardial infarction relative to satellite organs (e.g., kidney, liver, spleen, lungs).

### EXAMPLES

The following examples illustrate certain specific embodiments of the invention and are not meant to limit the scope of the invention. Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1. Enzyme-Responsive Nanoparticles for Targeted Accumulation and Prolonged Scaffold Retention in Heart Tissue after Myocardial Infarction

In this paper, we describe a method for targeting and retaining intravenous (IV) injected nanoparticles to the site of acute myocardial infarction (MI) in a rat model. Enzyme-responsive peptide-polymer amphiphiles (PPAs) were prepared and assembled into spherical micellar nanoparticles. The resulting nanoparticles respond to matrix metalloproteinases (MMP-2 and MMP-9) that are unregulated in heart tissue post-myocardial infarction. The nanoparticles undergo a morphological transition from spherical-shaped, discrete materials to network-like assemblies when acted upon by MMPs. We show that a 15-20 nm, responsive nanoparticle can be injected intravenously (IV), and undergoes reaction with MMPs in the heart after MI, with the resulting assemblies remaining within the infarct for up to 28 days. The initial studies reported here set the stage for the development of targeting systems for therapeutic delivery for acute MI. Critically, with this development, injection of materials is possible via the IV route, resulting in targeted accumulation and long term retention at the site of MI.

Heart failure following a myocardial infarction (MI) continues to be one of the leading causes of death.[1] Immediately after MI, there is an initial inflammatory response with cardiomyocyte death and degradation of the extracellular matrix. [2,3] This results in negative left ventricular (LV) remodeling that leads to wall thinning, LV dilation, and depressed cardiac function. [4] Several experimental approaches have been examined to inhibit this negative remodeling process; one promising approach is the use of injectable biomaterials,[5] which can be used as stand-alone scaffolds to encourage endogenous repair or for delivering therapeutics such as cells, growth factors, or small molecules. [5,6,7] Ideally a therapeutic should be delivered in a minimally invasive procedure as acutely as possible to prevent continued cardiomyocyte apoptosis and initiation of negative LV remodeling. Indeed, local intramyocardial delivery of biomaterials to the injured myocardium can be performed minimally invasively via catheter when the material is designed appropriately. [7] However, needle-based injection during the acute phase post-MI is unlikely to translate to the clinic given safety concerns with the weakened acute MI wall and unstable patient population.[7,8]

Nanoparticles are attractive for minimally invasive delivery because they may be administered via intravenous (IV) injection and target the heart through the enhanced permeability and retention (EPR) effect that is present in the acute stages of MI due to leaky vasculature. [9] Nanoparticle systems are unique in that they may be prepared with relatively small diameters ranging from 6-200 nm, making them ideal for systemic transport.[10,11] However, spherical nanoparticles at this length scale are often cleared from the body within 24 hours.[11,12] Targeted nanoparticle systems are likely to increase retention within the myocardium, but many of these studies lack long-term evaluation.[11,13,14] Both active targeting to the infarcted myocardium and retention of the material for periods longer than 1 week are current and significant challenges in developing nanoparticle delivery strategies to treat MI.

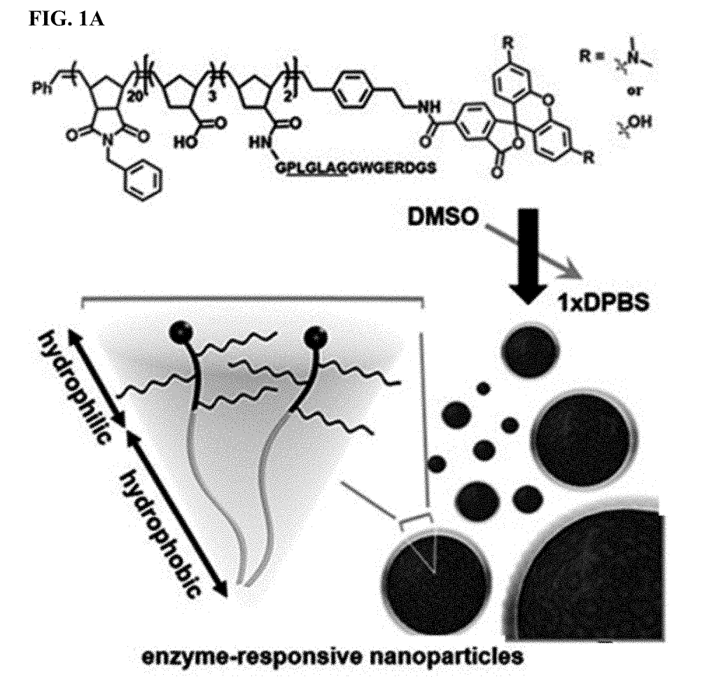

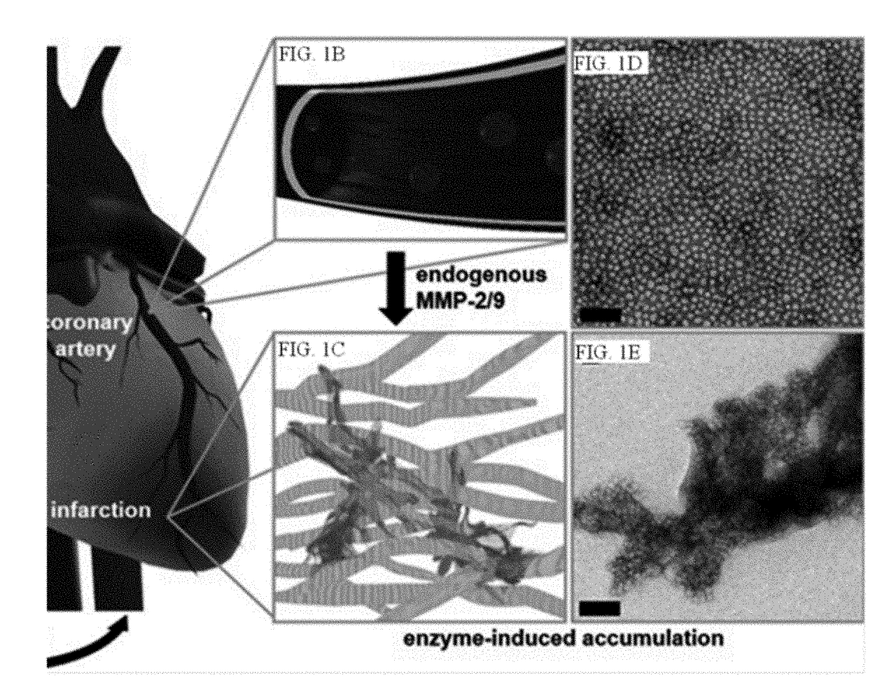

Provided herein are embodiments including a novel approach for targeting the MI as well for achieving prolonged retention of a material in an acute MI via IV injection. Nanoparticles (i.e. spherical micelles) were designed to respond to enzymatic stimuli (matrix metalloproteinases, MMPs) present in the acute MI resulting in a morphological transition from discrete micellular nanoparticles into network-like scaffolds (FIGS. 1A-1E). The fluorescent nanoparticle is composed of brush peptide-polymer amphiphiles (PPAs)[15] based on a polynorbornene backbone with peptide sequences specific for recognition of MMP-2 and MMP-9 (Methods and characterization provided following). IV delivery allows the enzyme-responsive nanoparticles to freely circulate in the bloodstream until reaching the infarct through the leaky post-MI vasculature,[9] where it assembles and remains within the injured site for up to 28 days post-injection. Previously, similar enzyme-responsive nanoparticles were shown to successfully accumulate after IV delivery in murine tumors that chronically overexpressed MMP-2 and MMP-9.[16] In this study, we demonstrate that enzyme-responsive nanoparticles target, assemble, and are retained in an acute MI, thereby providing a promising approach for delivery of therapeutics immediately post-MI and obviating the need for risky intramyocardial injections.

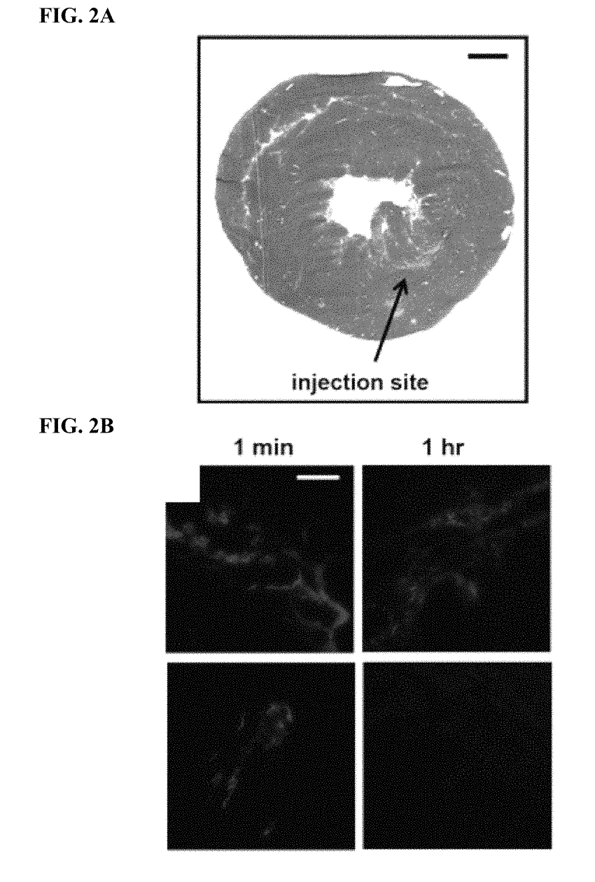

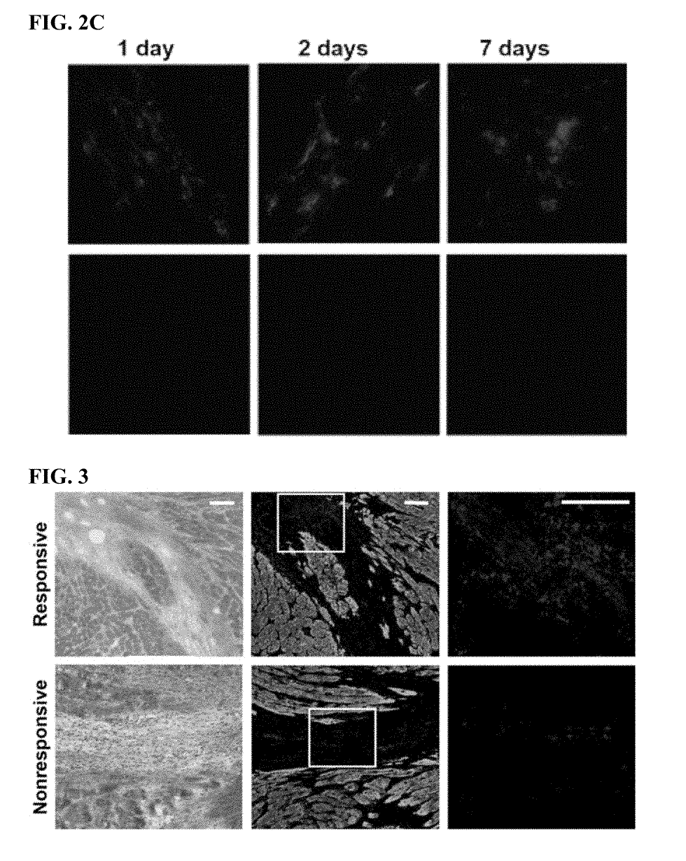

In embodiments, the nanoparticles respond to the upregulation of MMP-2 and MMP-9 that occurs in an acute MI,[17] creating a scaffold that would be retained in the tissue. To first evaluate whether the aggregated particles would remain in the infarct over time, we pretreated responsive (containing L-amino acids) and nonresponsive (containing D-amino acids) particles with MMP-9 for 24 hours to cause pre-aggregation of the responsive particles in vitro, and then injected the resulting material into healthy rat myocardium (see Supplemental Information). The results indicated that pretreated, responsive particles remain in the tissue up to 7 days post-injection, while nonresponsive particles were cleared after 1 hour (FIGS. 2A-2C). These data indicate that responsive particles are activated by MMP-9 to induce a morphological change to form an aggregate-like scaffold and are retained at the injection site. By contrast, nonresponsive particles remain inert in the presence of MMP-9 and are cleared quickly. Next, we evaluated our system in a rat MI model to determine whether upregulated MMPs in the infarct were sufficient to cause particle aggregation. Responsive and nonresponsive particles were delivered via intramyocardial injection 7 days post-MI, and rats were euthanized 6 days post-injection. Aggregation was observed in hearts that received responsive particles compared to minimal accumulation of the nonresponsive particles (FIG. 3), demonstrating that the increased expression of MMPs post-MI activates the morphological transition of our responsive particles. The responsive particles were observed up to 6 days post-injection, suggesting that the network-like scaffold remains in the infarct zone over time, compared to the minimal aggregation observed for nonresponsive particles.

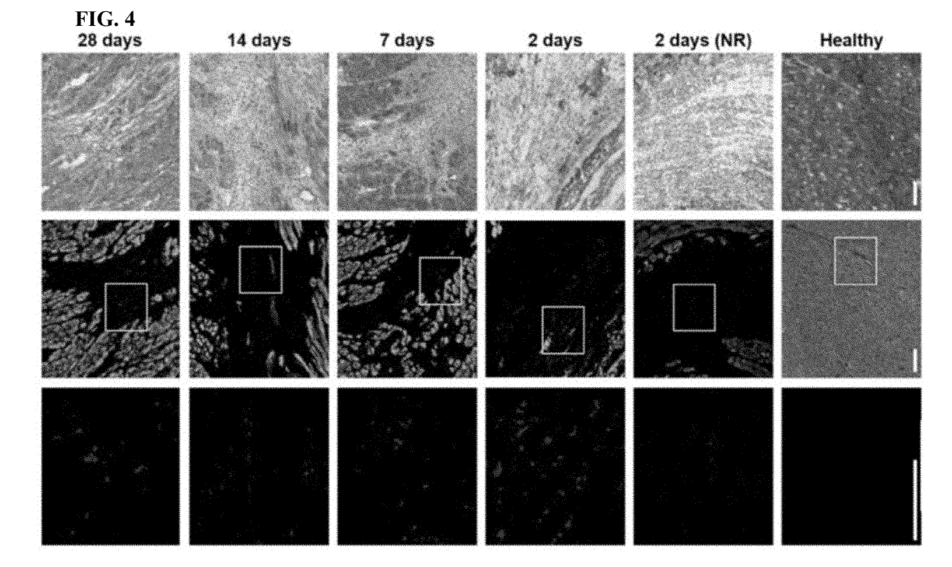

Finally, we tested whether the nanoparticle system could be delivered by IV and accumulate in the infarct. Responsive and nonresponsive nanoparticles (300 nmol) were injected into the tail vein of rats 24 hours post-MI (Methods provided in Supplemental Information). After 2 days, greater accumulation was observed in the hearts of animals that received the responsive particles, compared to the nonresponsive particles (FIG. 4). Particles were found in the infarct and adjacent borderzone, but not in the remote, viable myocardium. This pattern of accumulation continued to be observed in the tissue at 7, 14, and 28 days post-injection, demonstrating that this unique targeting approach provides for long-term retention in the tissue.[18] To test whether the leaky post-MI vasculature was necessary for particle accumulation in the infarct, we injected responsive particles (IV route) 30 days post-MI. The EPR effect, which occurs acutely post-MI,[3,19] is less prevalent over time.[9] For example, in other studies, less accumulation of liposomes has been observed when injected IV 7 days post-MI compared to both 1 and 4 days post-MI.[13] In our system, minimal accumulation was observed when delivered in the presence of a chronic MI (FIG. 11), suggesting that the EPR effect is necessary for particles to initially enter the tissue. Other nanoparticle systems have exploited the EPR effect for passive targeting including polymeric micelles [20], liposome based nanoparticles [21], and vascular endothelial growth factor (VEGF)-encapsulated liposomes.[22] These are limited by their lack of long-term retention. The MMP responsive nanoparticles reported here likewise use the EPR effect for initial passive targeting. However, by contrast, these particles undergo a morphological switch in response to upregulated MMPs, due to incorporation of the MMP-cleavable peptide sequence, which results in long-term retention (up to 28 days) at the site of infarction.

A therapeutic material should ideally remain in the infarcted tissue for a prolonged period of time (>1 week to several weeks) to adequately prevent negative LV remodeling. This places a substantial burden on nanoparticle design and delivery strategies. Only a limited number of studies have looked at long-term retention of nanoparticles, but of those that have, nanoparticles relying solely on passive targeting mechanisms (i.e. via the EPR effect) are rapidly cleared from target tissue. [23,24] Nanoparticles relying on active targeting mechanisms involving receptor recognition, such as MMP-2 and MMP-9 targeting peptides,[25] have demonstrated efficient targeting in ischemic zones and shown somewhat improved retention times from 24 hr to 7 days.[13,24] However, a receptor binding mechanism dependent on a dissociation constant of a targeting ligand for that receptor is not sufficient for prolonged retention in the infarct post-MI. Our results demonstrate that a localized morphology change from nanoscale spherical micelles to micron-scale network-like materials drastically enhances retention within the infarct as compared with non-responsive particles, providing a unique tactic for overcoming rapid tissue clearance. Unlike other systems, embodiments provided herein utilize an active targeting mechanism reliant on the second order rate constant of the enzyme, rather than a binding event. Furthermore, the morphological switch demonstrated by our system provides an assembly that may be maintained in the infarct zone. [25]

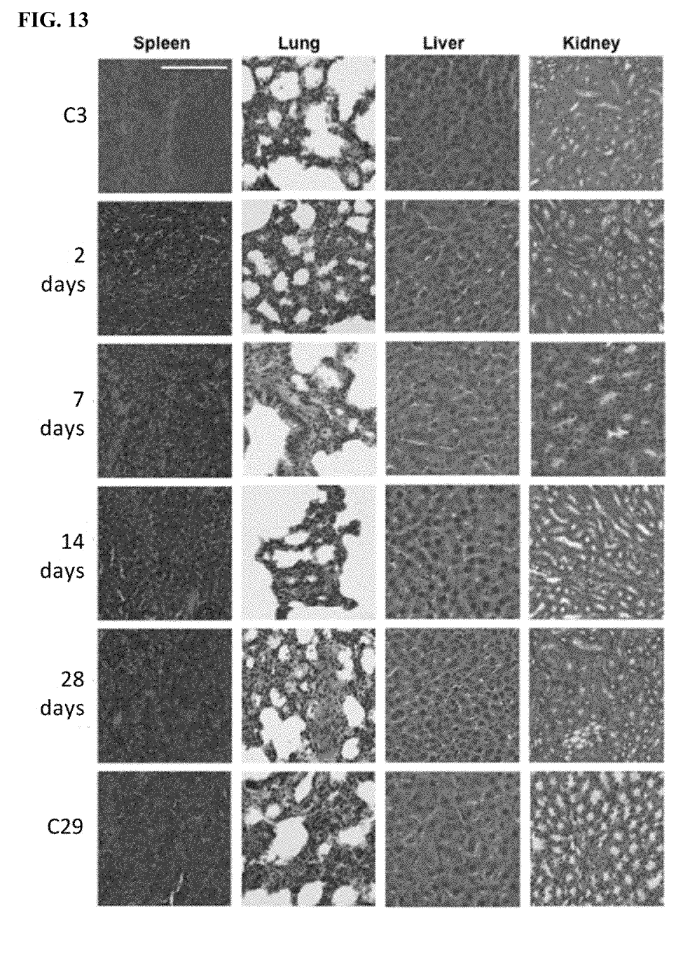

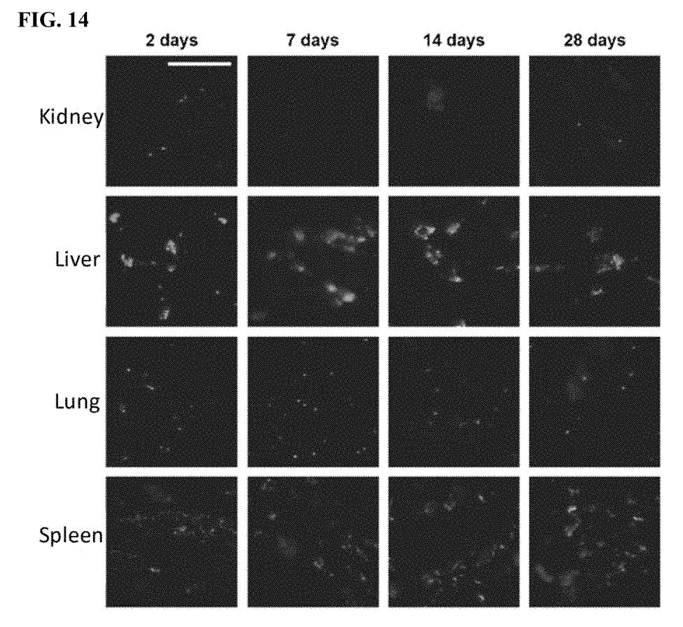

In addition to infarct targeting, off-site accumulation and safety are important to assess with any nanoparticle therapy. Biocompatibility of the responsive nanoparticles was investigated by an immunohistochemistry stain of CD68 (FIG. 12). As expected, CD68-positive cells were observed in the infarct only controls at 3 days post-infarction due to the inflammation response after injury. [26] We did not observe significant differences in the number of CD-68 positive cells in hearts that received either responsive or nonresponsive hearts, compared to the MI-only controls. We also evaluated the biodistribution of responsive particles to satellite organs. Particles were observed in the liver, spleen, and lung, with minimal presence seen in the kidneys (FIG. 13).[21,27] The biodistribution of nanoparticles when delivered IV has been described for a number of different systems, and it is generally accepted that larger particles are internalized by the reticuloendothelelial system (RES), while smaller particles are more widely distributed in the body.[10,28-30] The toxicity and biodistribution of nanoparticles when delivered IV is has been studied.[29,31] Similarly sized nanoparticles composed of silver, gold, and poly-ethylene glycol have shown different distribution trends. [28,30] Among polymeric nanoparticles, toxicity has been evidenced by acute liver inflammation and apoptosis.[28]

To assess the potential toxicity of our responsive particles, satellite organs from rats, 2 and 28 days post-IV injection (3 and 29 days post-MI, respectively) were evaluated and compared to those from both healthy and infarcted rats that received no injections (see FIG. 13). Histopathologic evaluation of the satellite organs was found to be normal. In addition, no signs of weight loss or changes in behavior were observed within both groups for up to 5 days post-injection and upon euthanasia 28 days post-injection. These results indicate that there were no signs of toxicity from the nanoparticles

Early intervention of MI has the potential to slow or inhibit the progression of negative LV remodeling. Current therapies to target the infarct include intramyocardial biomaterial injections[8], although translation to acute MI patients is unlikely given the increased risk of ventricular rupture immediately post-MI. [7.8] One promising, minimally invasive strategy is the systemic injection of nanoparticles. However, many of the investigated systems lack long-term retention within the MI[13,20,21] The enzyme-responsive nanoparticles described here, provide an efficient template for targeting to the acute MI and remain in the infarct for up to 28 days post-injection. We have shown that the responsive nanoparticles enzymatically respond and accumulate due to upregulation of MMPs after MI and are deliverable through both intramyocardial and IV injection. In summary, this unique approach includes a minimally-invasive method for the delivery of a material scaffold to infarcted myocardium, providing a promising approach for therapeutic delivery.

### Example 2. Materials and Methods

All reagents were purchased from Sigma-Aldrich and used without further purification. (N-Benzyl)-5-norbornene-exo-2,3-dicarboximide (1) was prepared as described previously.[1] 1-{[(2S)-bicyclo[2.2.1]hept-5-en-2-ylcarbonyl]oxy}-2,5-pyrrolidinedione (2) was prepared as described by Pontrello et al.[2] Dye-termination agents (3) were prepared as described previously. [3] (IMesH₂)(C₅H₅N)₂(Cl)₂Ru═CHPh was prepared as described by Sanford et al.[4] Polymerizations were performed under dry nitrogen atmospheres with anhydrous N,N-dimethylformamide (DMF). MMP-9 (human catalytic domain) (BML-SE360) was acquired from Enzo Life Sciences, as a 0.5 mg/mL solution at 24 U/μg in 50 mM TRIS (pH 7.5), 1 mM CaCl₂, 300 mM NaCl, 5 μM ZnCl, 0.1% BRIJ-35, and 15% glycerol.

Peptides were synthesized on an AAPPTec Focus XC peptide synthesizer. Analytical HPLC analysis of peptides was performed on a Jupiter 4u Proteo 90A Phenomenex column (150×4.60 mm) using a Hitachi-Elite LaChrom L-2130 pump equipped with UV-Vis detector (Hitachi-Elite LaChrom L-2420). Peptides were purified on a Armen Glider CPC preparatory HPLC. The solvent system for both HPLC instruments consist of (A) 0.1% TFA in water and (B) 0.1% TFA in acetonitrile. Mass spectra were obtained at the UCSD Chemistry and Biochemistry Molecular Mass Spectrometry Facility using a Micromass Quattro Ultima Electrospray Ionization (ESI) mass spectrometer. Polymer polydispersities and molecular weights were determined by size-exclusion chromatography on a Phenomenex Phenogel 5u, 1K-75K, 300×7.80 mm in series with a Phenomex Phenogel 5u, 10K-1000K, 300×7.80 mm (0.05 M LiBr in DMF) using a Shimatzu pump equipped with a Wyatt Technology DAWN-HELIOS multi-angle light scattering detector and a Hitachi L-2490 refractive index detector. Detectors were normalized with a 30,000 MW polystyrene standard. The hydrodynamic diameter (Dₕ) of particles was measured by DLS using a Wyatt Dynapro NanoStar. Zeta potential was measured with a Zetasizer Nano ZS90. TEM images were acquired on an FEI Tecnai G2 Sphera at 200 KV. Particle concentration measurements were conducted on an EnSpire Multimode Plate Reader.

Preparation of Peptide Substrates.

Peptides containing the amino acid sequence, GPLGLAGGWGERDGS, were synthesized, containing an MMP-2 and -9 recognition sequence (underlined). Peptides were synthesized on rink amide 4-methyl benzylhydrylamine (MBHA) resin via standard Fmoc-based solid phase peptide synthesis. Fmoc deprotection was performed by agitating resin in 20% 4-methylpiperidine in DMF for 5, draining, and repeating this procedure for another 15 min. Amino acid couplings were carried out for 45 min per amino acid using N,N,N,N′,N′-Tetramethyl-O-(1H-benzotriazol-1-yl)uranium hexafluorophosphate (HBTU) and N,N-diisopropylethylamine (DIPEA) (resin/amino acid/HATU/DIPEA 1:3:3:6). Final peptides were cleaved from resin by treatment with trifluoracetic acid (TFA), triisopropyl silane (TIPS), dithiothreitol (DTT), and water (TFA/TIPS/DTT/H₂O 88% v/v:2% v/v:5% w/v:5% w/w) for 2 hr. Peptides were then precipitated in cold ethyl ether and centrifuged at 4500 rpm for 15 min; this procedure was repeated twice. The precipitated peptide products were evaporated in vacuo to give an off white crude solid. Peptides were purified by reverse phase prep HPLC (0-15% Buffer B over 35 min, retention time=25 min), evaluated by negative-mode ESI MS (mass calculated [M−H]⁻=1425.69 m/z; mass observed [M−H]⁻=1425.58 m/z) and lyophilized to afford a pure white solid. L-amino acid responsive peptides and D-amino acid non-responsive controls were both synthesized by this method.

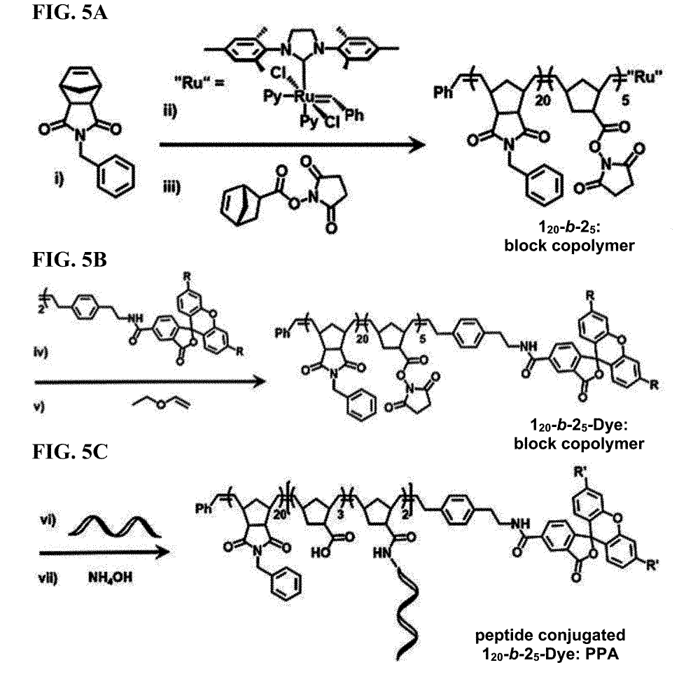

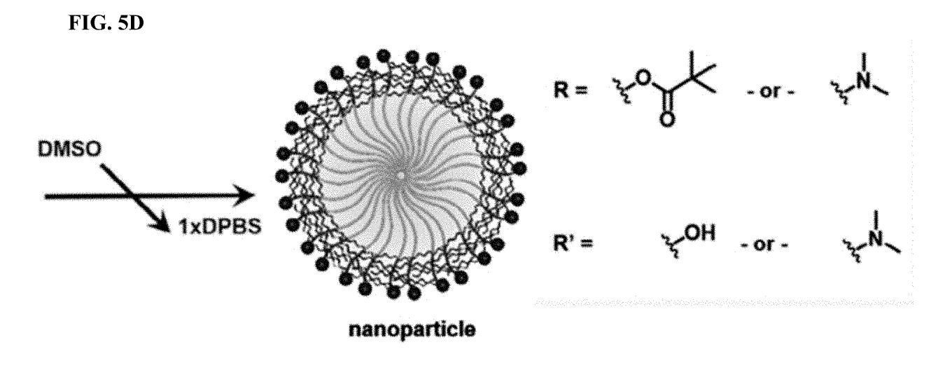

Nanoparticle formulation and synthesis schemes can be found in FIGS. 5A-5D. In brief, block copolymers were synthesized with a hydrophobic phenyl-moiety and a conjugatable N-hydroxy succinimide-ester prepared via ring-opening metathesis polymerization. [8] The living polymer was modified either with a fluorescein or rhodamine-labeled termination agent and further modified with a peptide sequence consisting of either L-amino acids as a cleavable substrate or D-amino acids as a nonresponsive substrate. Peptide polymer amphiphiles (PPA) were combined in a 1:1 molar ratio and dialyzed into 1×DPBS to create micellar nanoparticles.

Copolymer (1₂₀-b-2₅-Dye).

A block copolymer is also referred to herein as 1₂₀-b-2₅. To a stirred solution of i) (100 mg, 394.3 μmol) in dry DMF (2.0 mL) was added a solution of ii) the catalyst ((IMesH₂)(C₅H₅N)₂(Cl)₂Ru═CHPh) (14.3 mg, 19.7 μmol) in dry DMF (2 mL) under N₂ atmosphere. The reaction was left to stir under nitrogen for 45 min, after which an aliquot (20 μL) was removed and quenched with v) ethyl vinyl ether. After 15 min the quenched polymer was precipitated in anhydrous ethyl ether to give the homopolymer 120 as a solid. To the remaining reaction mixture, a solution of iii) (46.5 mg, 197.2 μmol) in dry DMF (2 mL) was added immediately following aliquot removal. The mixture was left to stir under nitrogen for 45 min, after which an aliquot (20 μL) was removed and quenched with v) ethyl vinyl ether. After 15 min the polymer was precipitated in anhydrous ethyl ether to give the block copolymer 120-b-2₅ as an off-white solid. The remaining bulk reaction mixture was split into two portions and a solution of iv) dye termination agent (16.3 mg Dye₁ or 13.6 mg Dye₂, 11.8 μmole) in dry DMF (1.0 mL) was added. The mixtures were left to stir under nitrogen for 2 hrs then v) ethyl vinyl ether (30 μL) was added to quench the catalyst. After 15 min the solutions were precipitated by dropwise addition to cold anhydrous ethyl ether to give the two copolymers, 1₂₀-b-2₅-Dye₁ and 1₂₀-b-2₅-Dye₂.

Synthesis of PPA-F and PPA-R.

To a stirred solution of copolymer 1₂₀-b-2₅-Dye₁ or 1₂₀-b-2₅-Dye₂ (18.7 mg, 2.896 μmol) dissolved in a dry mixture of 4:1 DMF:DMSO (370 μL) at 50 mg/mL was added L-amino acid peptide and DIPEA (copolymer:peptide:DIPEA 1:4:16). The reaction was stirred at room temperature for 27 hrs. For 1₂₀-b-2₅-Dye₁, NH₄OH (460 μL, 160 μL/μmol polymer) was added and stirred for 45 min at room temperature to deprotect the fluorescein pivalate groups, followed by evaporative removal of NH₄OH. Solutions were then precipitated in cold anhydrous ether and washed with cold methanol (2×4 mL) to obtain the peptide conjugate of the 1₂₀-b-2₅-Dye systems. Precipitate was collected by centrifugation at 13,000 rpm for 3 min followed by decanting. The precipitated product was then dried to afford the peptide conjugated PPA-F and PPA-R (Dye₁ or Dye₂ terminated polymers of 1₂₀-b-2₅). Block copolymers, also referred to herein as PPA-F or PPA-R, are a plurality of Dye₁ or Dye₂ terminated polymers of 1₂₀-b-2₅. This same procedure was followed for D-amino acid analogues.

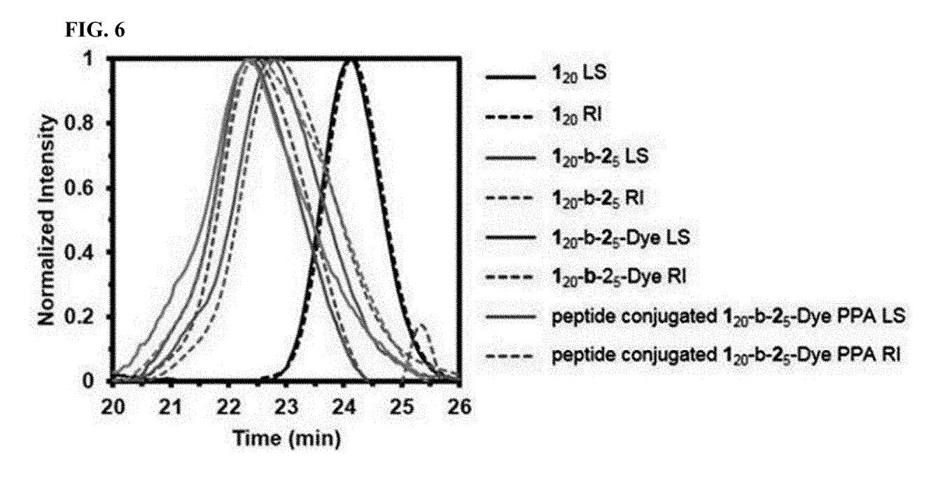

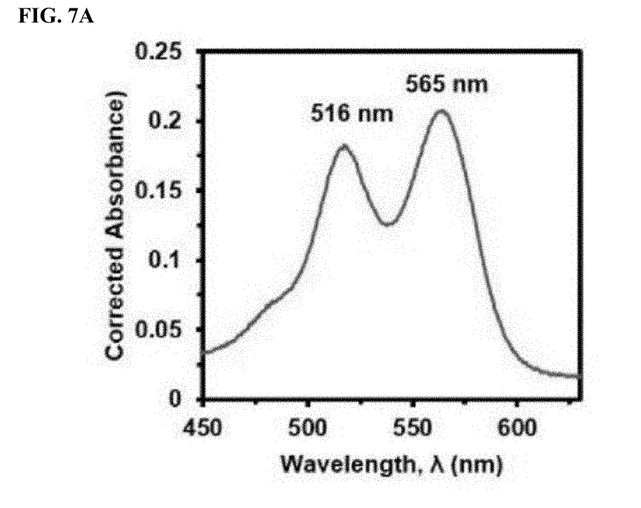

FIG. 6 depicts SEC-MALS intensity plot of polymers 1₂₀, 1₂₀-b-2₅, 1₂₀-b-2₅-Dye, and peptide conjugated 1₂₀-b-2₅-Dye PPA. Light scattering (LS) and differential refractive index (RI) traces are show as solid and dashed lines, respectively.

Polymer Characterization.

Representative SEC-MALS data for the 1₂₀ homopolymer, 1₂₀-b-2₅ copolymer, 1₂₀-b-2₅-Dye₁ copolymer, and 1₂₀-b-2₅-Dye₁ PPA were collected (FIG. 6). The do/dc for these polymers is 0.179. Calculations of polymer sizes, their PDI, and block size or conjugation number, are provided in Table 1 following. Calculations for 1₂₀-b-2₅-Dye₂ are not provided due to interference of the dye at λ=657 nm when measuring the differential refractive index (dRI). The degree of peptide conjugation for 1₂₀-b-2₅-Dye₂ is assumed to be the identical to that of 1₂₀-b-2₅-Dye₁.

TABLE 1
Polymer molecular masses (g/mol) with respect to number (Mₙ) and
weight (M_{w}), and polydispersity indices ± standard deviation,
as measured by SEC-MALS. Calculated block sizes or peptide
conjugation numbers are shown.

| | | | | peptide conjugated |
|---|---|---|---|---|
| | 1₂₀ | 1₂₀-b-2₅ | 1₂₀-b-2₅-Dye | 1₂₀-b-2₅-Dye₁ |
| Mₙ | 5253 (±1.512%) | 6461 (±1.829%) | 7192 (±1.833%) | 8663 (±1.919%) |
| M_{w} | 5310 (±1.447%) | 6873 (±1.543%) | 7465 (±1.833%) | 9583 (±1.726%) |
| PDI | 1.011 (±2.093%) | 1.064 (±2.393%) | 1.038 (±2.533%) | 1.106 (±2.581%) |
| size or number | 20 | 5 | n/a | 2 |

Spherical Micelle Formulation.

Polymeric micelles, also referred to herein as responsive particles, were prepared using equal parts PPA-F and PPA-R containing L-amino acid peptides whereas nonresponsive particles were prepared using equal parts PPA-F and PPA-R containing D-amino acid peptides. In other words, a polymeric micelle includes a plurality of the block copolymers as described herein. PPA-F (25 mg, 2.89 μmol) and PPA-R (25 mg each, 2.89 μmol each) were dissolved in DMSO (50 mL, 1 mg/mL) and stirred as H₂O was slowly added via syringe pump at 0.75 mL/hr until the solution was 30% v/v aqueous (critical water concentration) and left to stir for 2 days. H₂O was then added at 5 mL/hr until reaching 50% v/v aqueous concentration. This solution was transferred to 3,500 MWCO SnakeSkin dialysis tubing and placed in water (pH 8.0). The buffer was changed three times per day for 2 days. The solvent was then switched to 1×DPBS using the same buffer exchange conditions listed above.

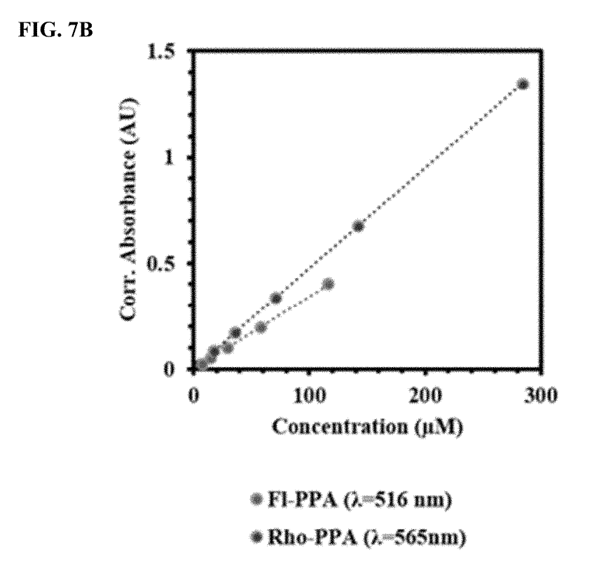

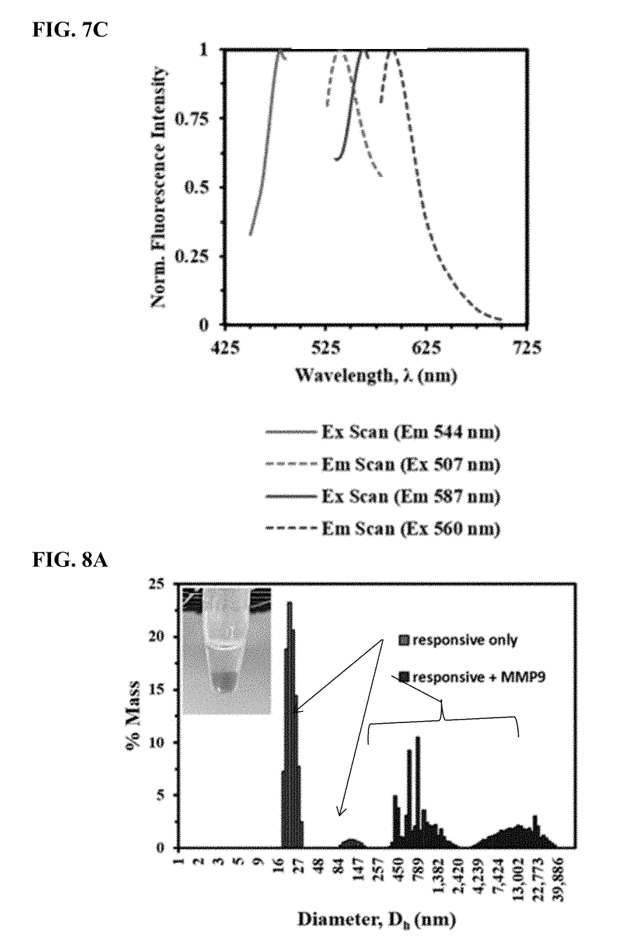

Stock Solution Preparation and Concentration Characterization (FIGS. 7A-7C).

Particles were concentrated using EMD Millipore Amiccon Ultra-15 centrifugal filter units (10K Nominal Molecular Weight Limit, NMWL). Filters were centrifuged at 3000 rcf for 8 min at 21° C. Final stock solutions were sterilized through 0.2 μm EMD Millipore Steriflip sterile disposable vacuum filter units. Stock solution concentrations were determined by disrupting and diluting nanoparticles 7-fold in DMSO and recording absorbance values for fluorescein and rhodamine. Plate reader measurements for standards and stock solutions were performed with 150 uL of sample in clear bottom 96-well plates. Nanoparticle concentrations are reported in this paper with respect to calculated total dye concentration.

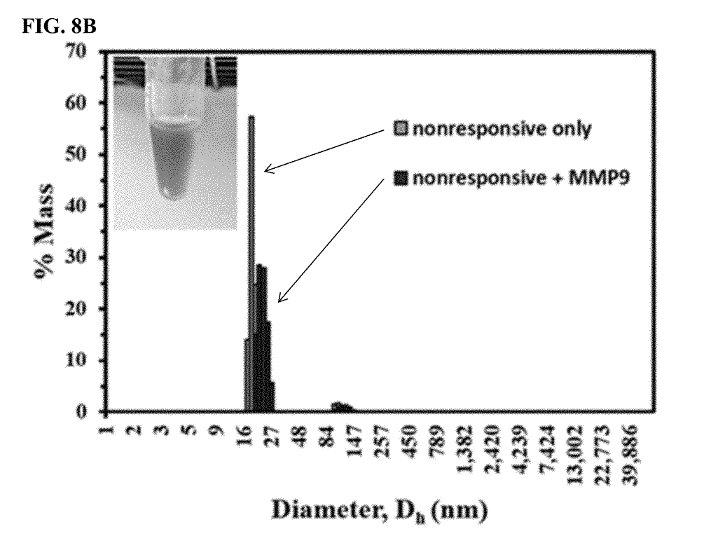

Dynamic Light Scattering (DLS) and Zeta Potential (FIGS. 8A-8B, and Table 2).

The hydrodynamic diameter (Dₕ) of responsive (containing L-amino acid peptides) and nonresponsive (containing D-amino acid peptides) particles with and without MMP-9 treatment was calculated by DLS (FIGS. 8A-8B). Only responsive particles exhibit a morphology switch from monodisperse micellar nanoparticles (15-20 nm) to micron-scale (0.7-14 μm) scaffolded aggregates. Visible aggregation as a result of 24 hr MMP-9 treatment for responsive particles can be seen with the eye in the inset picture in the figure herein. In contrast, nonresponsive particles remain intact by DLS and fully dispersed in solution. Table 2 below provides quantitative data on particle diameter with respect to calculated % mass and % intensity, as well as the measured % polydispersity (Pd). Zeta potential of 30 mM solutions of nanoparticles was measured in H₂O (pH 7.4) at 37° C., and found to be −56±2 mV (n=12 samples).

TABLE 2
DLS measurements of particle diameter (nm) before
and after incubation with MMP-9. Provided are % mass,
% intensity, and % polydispersity of particle sizes. Two
major populations exist for responsive polymeric micelles
treated with MMP-9.

| | Diameter (nm) | % Mass | % Intensity | % Pd |
|---|---|---|---|---|
| Responsive | 23.0 | 94.6 | 26.4 | 12.1 |
| polymeric micelles | 127.0 | 5.4 | 73.6 | 17.9 |
| Responsive | 615.0 | 58.9 | 75.0 | n/a |
| polymeric micelles + | 14075.7 | 40.3 | 5.5 | n/a |
| MMP-9 | | | | |
| Nonresponsive polymeric | 18.5 | 96.1 | 23.3 | 5.0 |
| micelles | 104.0 | 3.9 | 77.7 | 6.1 |
| Nonresponsive polymeric | 22.8 | 97.9 | 24.8 | 9.1 |
| micelles + MMP-9 | 117.7 | 2.1 | 75.2 | 10.6 |

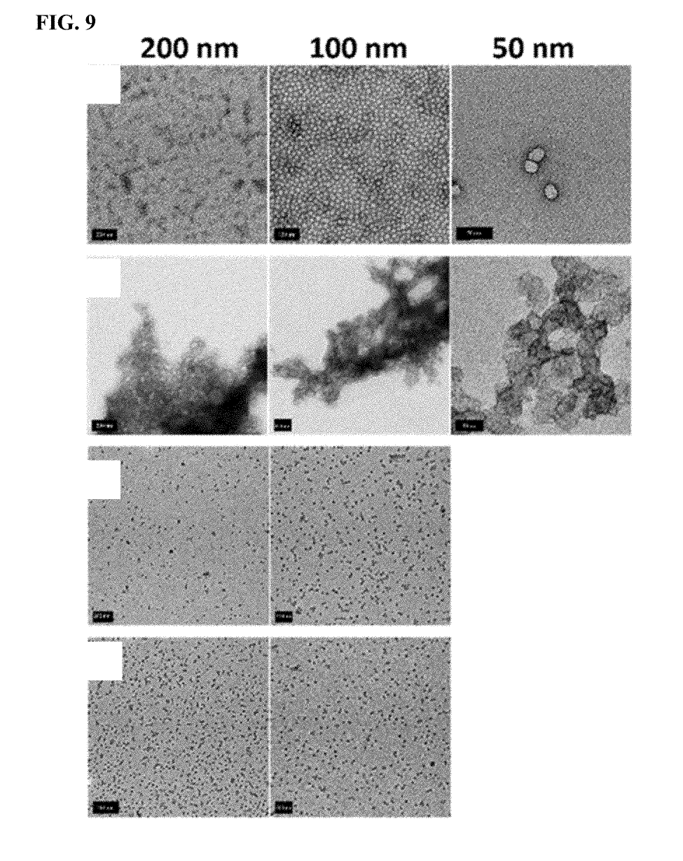

Transmission Electron Microscopy (TEM) (FIG. 9).

Small aliquots (3.5 μl) of sample were utilized for dry state TEM via standard procedures. Briefly, the sample was applied onto 400 mesh carbon grids (Ted Pella, INC.) that had previously been glow discharged using an Emitech K350 glow discharge unit and plasma-cleaned for 90 s in an E.A. Fischione 1020 unit. After 3-5 min, samples were rinsed with water and stained with 1% (w/v) uranyl acetate briefly and wicked dry. The sample grid was then transferred into a grid holder in the FEI Sphera microscope. Micrographs were recorded on a 2K×2K Gatan CCD camera. Only responsive nanoparticles that were treated with MMP-9 exhibited a morphology switch from discrete micellar nanoparticles to micron-scale scaffold aggregates.

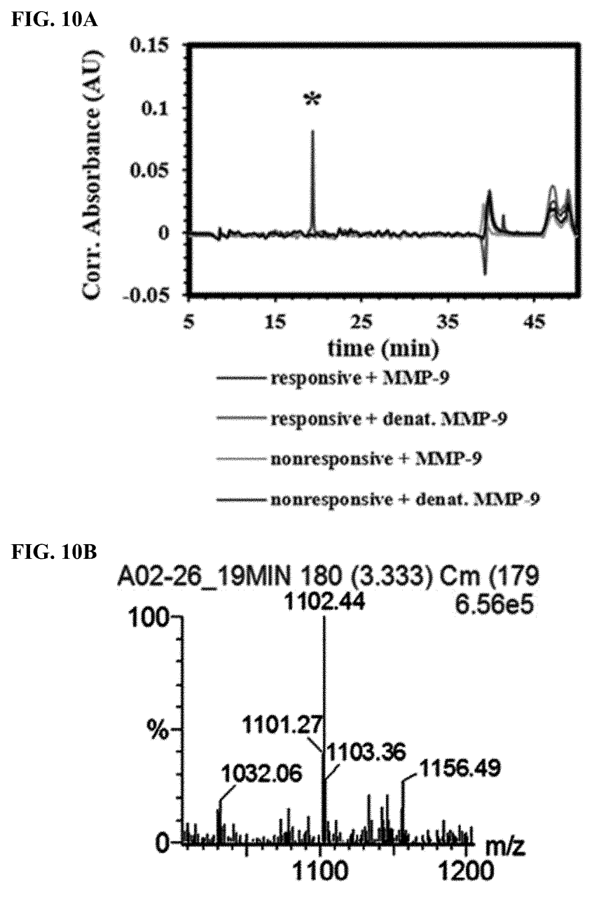

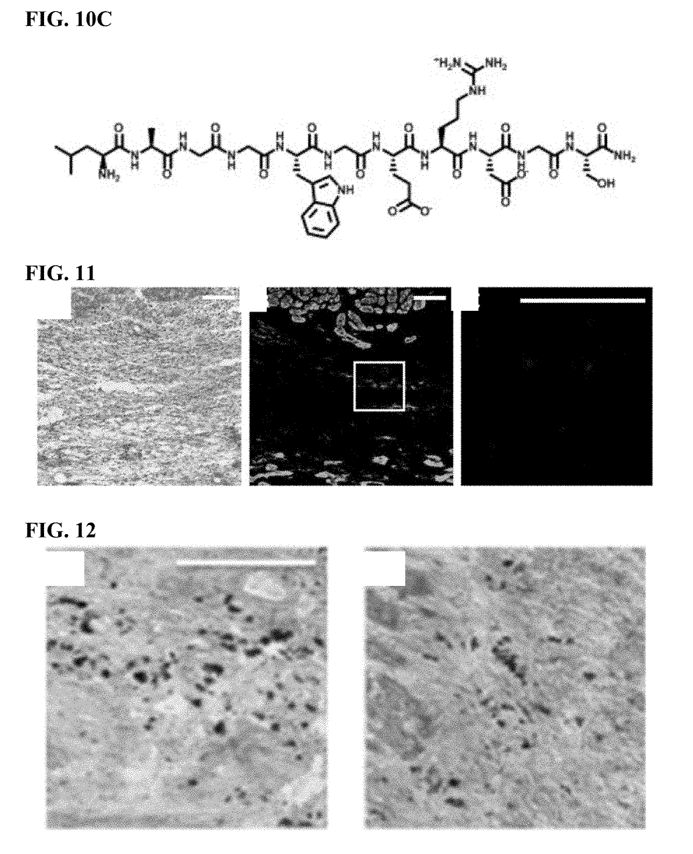

MMP-9 Cleavage Assay—In Vitro (FIGS. 10A-10C).

Micellar nanoparticles (100 μM, with respect to total dye) were treated with MMP-9 (1 μM) for 24 hr at 37° C. in cleavage buffer (200 mM NaCl, 50 mM TRIS, 5 mM CaCl₂, and 1 mM ZnCl₂, pH 7.5). Denatured enzyme controls were prepared by heating the enzyme solution to 65° C. for 30 min before adding to the substrate. Analytical HPLC was used to monitor enzymatic cleavage of peptide substrate within nanoparticles. In FIG. 10A, the expected cleavage product at Rₜ=19 min (red trace) is produced when responsive nanoparticles are treated with MMP-9. The cleavage product mass-to-charge ratio was measured by ESI in negative ion mode to be [M−H]⁻ 1102.44 m/z (calculated 1102.15 m/z) (FIG. 10B) which corresponds to the predicted peptide sequence when cleaved between the G and L amino acid residues of our substrate, observed in FIG. 10C.

Animal Studies.

All animal experiments were conducted in accordance with the guidelines established by the Institutional Animal Care and Use Committee at the University of California, San Diego and the American Association for Accreditation of Laboratory Animal Care and approved by the Institutional Animal Care and Use Committee at UCSD (A3033-01). Female Sprague Dawley rats (225-250 g) were used in all studies.

Intramyocardial Injection Surgery.

An intramyocardial injection was performed under 5% isofluorane, using a previously described procedure. [5,6] To investigate the clearance of responsive and nonresponsive particles (i.e. the polymeric micelles comprised of block copolymers), the particles (2.8 nmol, 70 μl) were pre-treated with MMP-9 catalytic domain (1 nmol) for 24 hours at 37° C. prior injection. Aggregated responsive particles and unchanged nonresponsive particles were injected into the left ventricular free wall of female Sprague Dawley rats (225 to 250 g) with a 27 G needle. Animals were euthanized with an overdose of pentobarbital (200 mg/kg) 1 minute, 1 hour, 1 day, or 7 days post-injection (n=3 per group).

Myocardial Infarction (MI) and Intramyocardial Injections.

MI was performed via 25-min ischemia-reperfusion, as previous described.[5,7] The intramyocardial injections were performed 7 days post-MI, as described above. The particles were prepared in sterile saline (75 nmol, 75 μl) and injected to the myocardium with a 27 G needle. Animals were euthanized with an overdose of pentobarbital (200 mg/kg), and the heart was collected for histological analysis.

Myocardial Infarction (MI) and Tail Vein Injections.

MI was performed via 25-min ischemia-reperfusion, as previous described.[5] Tail vein injections were performed 24 hours post-MI, and animals were anesthetized with 5% isoflurane and maintained at 2.5% isoflurane during the procedure. Animals received 1 mL of responsive or nonresponsive nanoparticles (n=3 per group, 300 μmol) and were monitored daily over at least 5 days. All animals were visually examined and weighed prior intravenous injection and euthanization. Animals were euthanized with an overdose of pentobarbital (200 mg/kg), and the heart, lung, liver, spleen, and kidneys were collected for histological analysis. H&E stained slides of the heart, liver, lung, kidney, and spleen were examined by an experienced histopathologist who was blinded to the groups. Healthy, untreated organs were used as a positive control.

Histology and Immunohistochemistry.

After euthanasia, hearts were resected and fresh frozen in OCT. Using a cryostat, short axis cross-sections were taken every 250 μm from the apex to the base throughout each heart. Sections within each area were stained with hematoxylin and eosin (H&E) to identify the infarct region or mounted with FluoroMount for fluorescent imaging using Axiovision software. Tissue slides stained via immunohistochemistry using anti-α-actinin (Sigma-Aldrich) were imaged using fluorescent scans captured with a Leica Ariol Platform. Tissue slides stained for CD68 positive cells were prepared using a mouse anti rat antibody (AbD Serotec) with 1:100 dilution. Samples were then incubated with 3% H₂O₂ for 30 min, horseradish peroxidase (HRP) conjugated goat anti-mouse IgG at 1:100 dilution for 30 min, visualized with diaminobenzidine (DAB) (Vector Laboratories), and then examined under a light microscope.

Satellite Organ Preparation.

The organs were embedded in formalin for 48 hours and transferred into 70% ethanol prior to paraffin embedding. Organs oriented both laterally and longitudinally. Using a microtome, short axis cross-sections were taken every 500 μm. Sections within each area were stained with hematoxylin and eosin (H&E) upon deparraffization or mounted with FluoroMount for fluorescent imaging using Axiovision software.

### Example 3. Therapeutic Enzyme-Responsive Nanoparticles for Targeted Delivery and Accumulation in Tumors

In this example, the applicants describe an enzyme-responsive, paclitaxel-loaded nanoparticle and assess its safety and efficacy in vivo in a human fibrosarcoma murine xenograft. The material was generated via graft-through block copolymerization of norbornene monomers with hydrophilic targeting peptides together with hydrophobic paclitaxel prodrugs. This work represents a study demonstrating the utility of enzyme-responsive nanoscale drug carriers capable of targeted accumulation and retention in tumor tissue in response to overexpressed endogenous enzymes. Critically, we observed low systemic toxicity in healthy mice following intravenous administration, with the maximum tolerated dose (MTD) exceeding 240 mg/kg with respect to paclitaxel. Furthermore, we observed efficacy against tumorigenesis paralleling that of paclitaxel at equivalent intravenous dosing, and near complete tumor growth suppression when administered intratumorally. This work represents a significant departure from traditional targeted drug delivery systems and presents a new avenue of exploration for nanomedicine.

One goal of nanomedicine is to treat disease through selective accumulation of therapeutics in diseased tissue. Nanoparticles offer the potential to package large quantities of drug cargo per carrier entity, to be decorated with targeting moieties in a multivalent fashion, and to have the potential to decrease off-target effects associated with conventional treatment regimes, while simultaneously increasing efficacy.[1] With respect to cancer therapy, the enhanced permeability and retention (EPR) effect has been implicated, albeit somewhat controversially,[2] as a mechanism by which nanomaterials accumulate in the fenestrated vasculature of tumor tissue. However, among other factors, this effect is limited to diseases that undergo rapid angiogenesis in their pathology. [3] Furthermore, the EPR effect is a passive mechanism of accumulation. To achieve active targeting, nanoparticle drug carriers have utilized receptor-mediated endocytosis[4] and hence, rely on the overexpression of surface receptors on disease-associated cells. Therefore, researchers have focused on a recurring set of ligand-receptor combinations, including RGD with integrin αᵥβ₃,[5] NGR with aminopeptidase N,[6] and folic acid with the folate receptor. [7]

We envisioned a different targeting method, wherein an enzymatic signal endogenous to tumor tissue directs a build-up of material selectively at the tumor site. [8, 9-11] Specifically, we aimed to utilize matrix metalloproteinases (MMPs), overexpressed in an array of cancer types and present as catalytic, extracellular or membrane-bound tumor markers.[12] In this strategy, nanoparticles have shells decorated with peptides containing a substrate for MMPs. Upon exposure to the enzyme, the materials undergo a nano- to microscale change in size, coupled with a change in morphology.[10] In this way, the tumor guides the accumulation process through MMP expression patterns resulting in active accumulation through catalytic amplification. To date, we have demonstrated this targeting method for the accumulation of fluorescent probes with the aim of developing approaches for guided surgery[13] and for diagnostic purposes.[9-11]

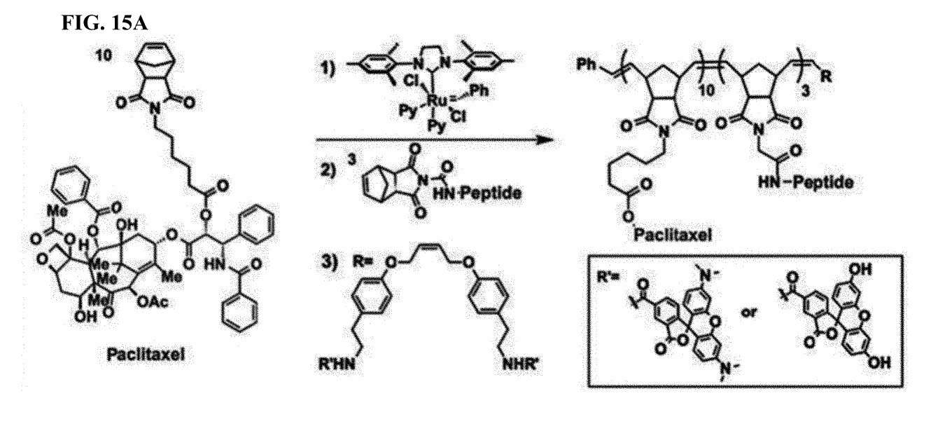

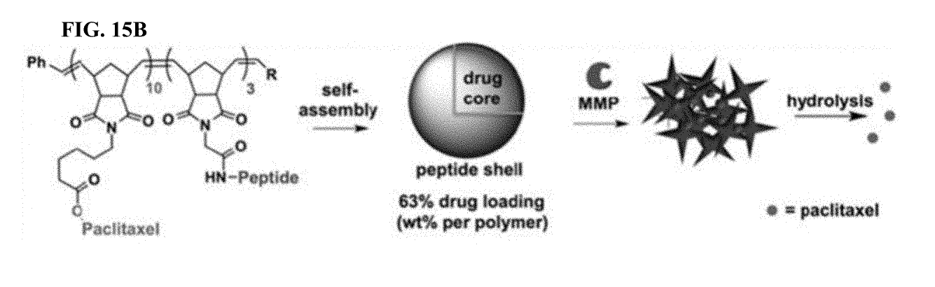

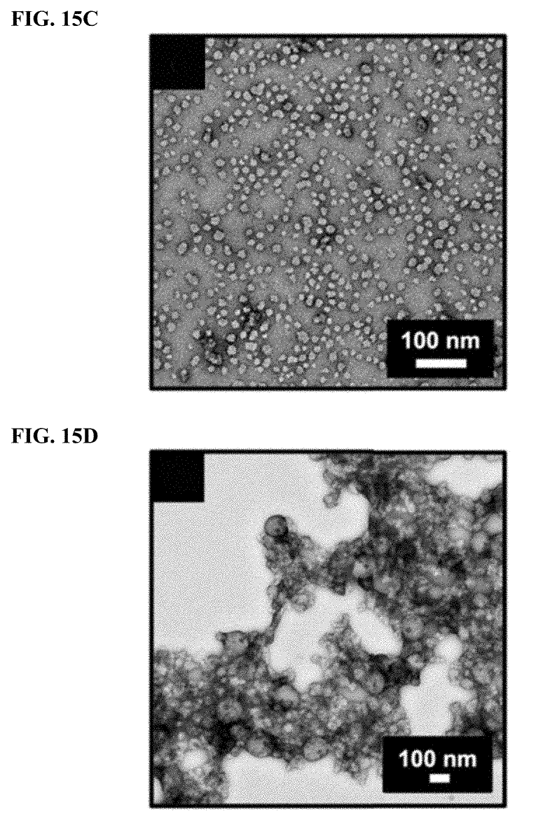

Given our experience with targeting fluorescent probes, we developed an MMP-targeted nanoparticle platform that could be employed as a tool for the delivery of chemotherapeutics (FIGS. 15A-15C). Towards this end, we generated micellar nanoparticles through the direct diblock copolymerization of a novel paclitaxel conjugate with a MMP substrate (FIG. 15A). Both functional monomers were synthesized as norbornene analogues amenable to ring opening metathesis polymerization (ROMP),[14, 15, 16] utilizing a highly functional group tolerant Ru-based initiator[15, 17, 18] capable of producing polymers with low dispersity in a highly reproducible manner. [18] The resulting block copolymers assemble into micellar nanoparticles with a surface comprised of shell of MMP-substrates and a hydrophobic paclitaxel core (FIG. 15B). Notably, the drug is polymerized directly and is covalently bound via a biodegradable ester linkage. Upon exposure to MMP, the peptide shell is cleaved and the nanoparticles undergo a drastic change in morphology from discrete, spherical micelles 20 nm in diameter to form micron-scale assemblies visualized by transmission electron microscopy (FIGS. 15C-15D).[10, 11] This transition amounts to a tumor-guided implantation of the polymer-bound drug conjugate via intravenous (IV) injection.

We utilized paclitaxel (PTX) in the hydrophobic block of the copolymer and as the therapeutic moiety in this motif, as it is a potent microtubule-stabilizing agent[19] and standard component of chemotherapy regimes for many malignant and metastatic cancers. The free 2′-hydroxyl group of PTX is absolutely required for its antitumor activity[20], but is available for conjugation via a biodegradable ester formed with a carboxylic acid-functionalized norbornene (PTX-norbornenyl ester). This ensures PTX is completely inactivated, and thus is delivered as a prodrug prior to hydrolysis from the polymer scaffold. The peptide sequence GPLGLAGGERDG (SEQ ID NO:4) was employed as the hydrophilic moiety and MMP recognition sequence. The sequence was amenable to graft-through polymerization affording precise control of the polymer chemistry and subsequent enzymatic response.[16, 21]

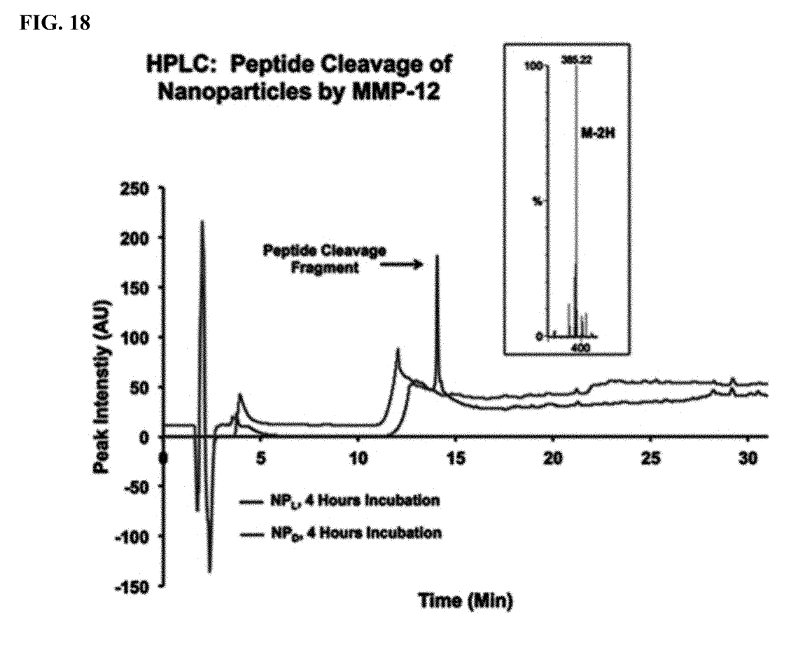

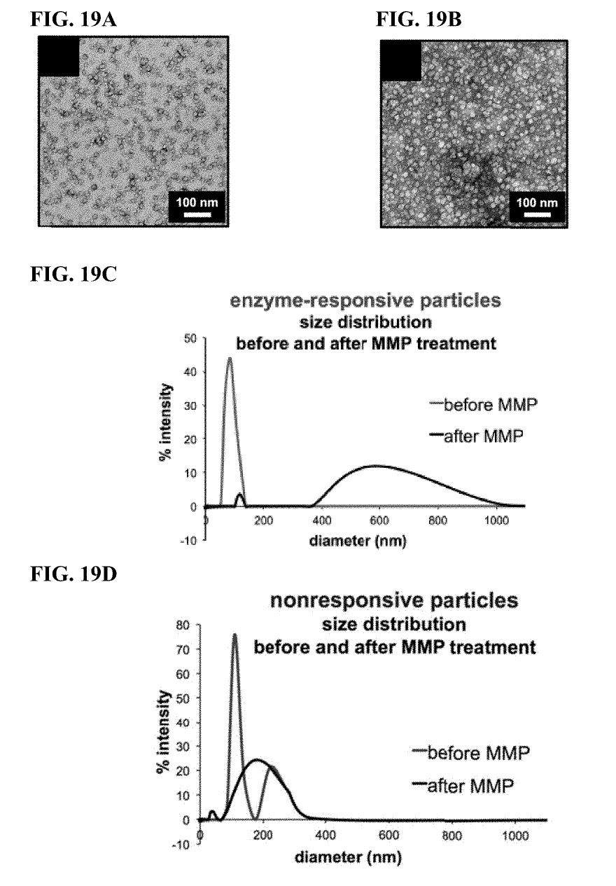

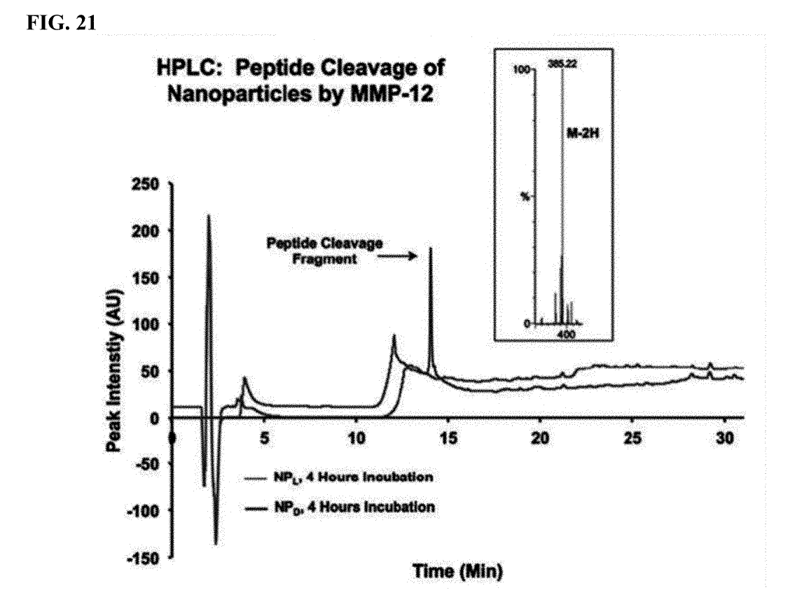

Uniform nanoparticles with high drug loading (63% by weight per polymer) spontaneously assembled upon dialysis of the copolymers initially dissolved in DMSO against aqueous solution. Two analogous systems whose hydrophilic blocks were comprised of either all L- or all D-amino acid peptides, were generated to afford enzyme-responsive or nonresponsive, negative control nanoparticles respectively. Additionally, both systems were split into two batches during polymerization of the second block, and terminated with either fluorescein or rhodamine, which form a FRET pair when formulated into a single particle[9, 10] to enable tracking of these materials in vivo. With both responsive (NPL) and nonresponsive (NPD) nanoparticles in hand, we confirmed the ability of these materials to respond to MMP and aggregate in vitro (FIG. 18, FIG. 19, and FIG. 21) In summary, catalytic exposure of NPL to MMP-12 for 4 hours led to the aggregated material. Conversely, NPD showed no change in structure when exposed to the same conditions. On the basis of these observations, we hypothesized that NPL would collect within tumor tissue upon IV injection, or be retained following intratumoral (IT) injection. This would lead, in turn, to release of PTX within the tumor tissue achieving a measurable therapeutic dose via hydrolysis induced by the tumor microenvironment. By contrast, NPD would not be retained, but rather clear from the tumor tissue, before PTX hydrolysis and release could lead to a therapeutic dose.

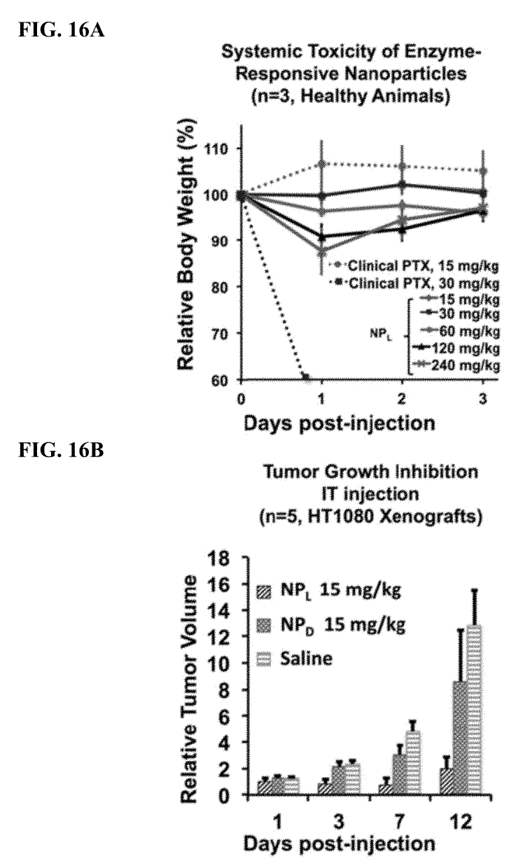

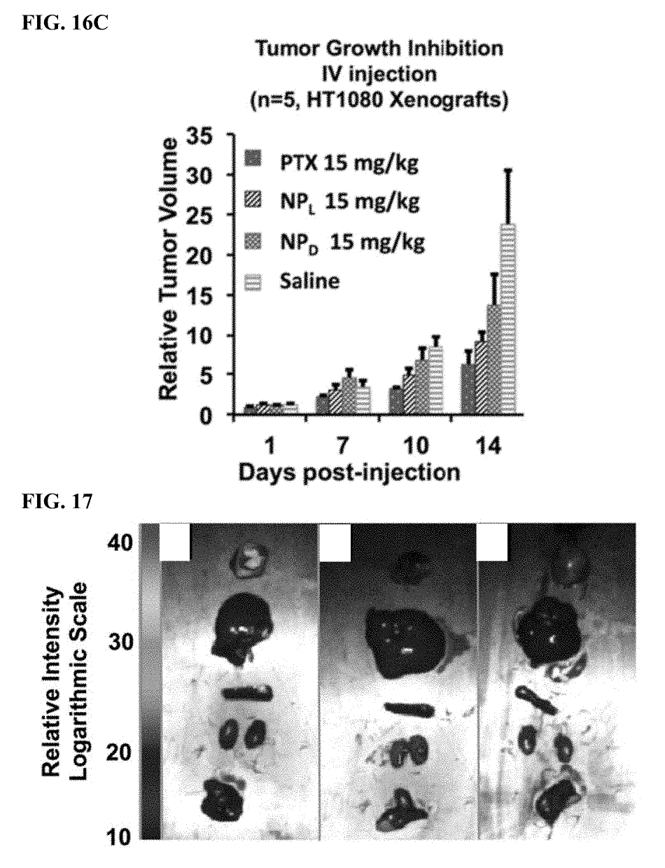

We examined the safety and efficacy of PTX-loaded NPs in three proof-of-concept in vivo studies (FIGS. 16A-16C); 1) maximum tolerated dose (MTD) following IV administration 2) efficacy post-IT injection and 3) efficacy post-IV injection. For these studies, employed an HT-1080 fibrosarcoma xenograft cancer model known to overexpress MMPs[22] and to rapidly proliferate in a predictable manner after subcutaneous implantation. All animal procedures were approved by the University of California, San Diego's institutional animal care and use committee (IACUC).

To examine the safety of our system, an MTD was determined in healthy nu/nu mice. In animal models, toxicity was secondarily measured as a function of animal body weight,[23] with lethality and/or weight loss of greater than 20% suggestive of severe adverse reactions. The MTD in mice of clinically formulated PTX as a suspension in 1:1 Cremophor EL (polyoxyethylated castor oil) to ethanol has been previously established as being in a range between 10-30 mg/kg. [24] In our hands the clinical formulation had a MTD of 15 mg/kg when administered via single tail-vein IV injection. Conversely, we were able to administer NPL via tail vein IV at a dose of 240 mg/kg. Therefore, NPL exhibited a MTD 16 times greater than PTX without overt clinical toxicity, except for a 10% weight loss at 1 day with slow recovery over the next 3 days (FIG. 16A). This suggests our enzyme-responsive materials are safely administered, even at exceptionally high doses. To examine efficacy, NPL was tested against NPD, clinically formulated PTX, and saline in a series of IV and IT studies, with all injection concentrations standardized to the equivalent of a 15 mg/kg dose of PTX. In brief, tumor xenografts of HT-1080 were established by inoculating each mouse subcutaneously with ˜10⁶ cells. Drug treatments were initiated once the tumors reached approximately 50 mm3 in size. Tumor growth was assessed by daily measurement of tumor diameter through B-Mode Ultrasound (US) [25].

To confirm that morphology change is necessary to retain our materials and further, to determine whether this accumulation event leads to a release of drug cargo at the tumor site, we conducted an efficacy study in which the effect on tumor growth of NP_{L} was compared to that of both NP_{D} and saline (negative control) following IT injection. Live-animal fluorescence imaging was used to monitor the retention of our materials post-injection as a function of FRET (Förster Resonance Energy Transfer) signal with the eXplore Optix preclinical scanner (λₑₓ=470 nm and λₑₘ=590 nm). Briefly, PTX-nanoparticles contained both fluorescein- and rhodamine-labeled polymers, as these molecules form a FRET pair. We monitored the presence of a viable FRET signal by exciting the donor at 470 nm and monitoring the emission of the acceptor at 590 nm. FRET is only manifest when donor and acceptor molecules are within the Förster radius, as they are in these materials. The use of a FRET signal, rather than a single-dye system, eliminates much of the background signal caused by autofluorescence. As shown in FIG. 17, FRET is observable up to 5 days following IV injection of NP_{L}, suggesting that these materials are accumulating and being retained over a long time-scale. Importantly, FRET is only observed for the first 5 hours following IV injection of NP_{D}, indicative of rapid clearance of the material, presumably due to the lack of MMP-induced morphology change. Excitingly, we observed superior tumor growth suppression by NP_{L} up to 12 days post-injection, and in fact, one animal in the cohort experienced complete remission beyond two months post-treatment (FIG. 16B). Conversely, there is no observable difference between NP_{D} and saline throughout the duration of the study. These results provide evidence that morphology change is required for the function of these materials.

Further evidence of efficacy was elucidated through a preliminary IV study. The effect on tumorigenesis of NP_{L} was compared to that of clinically formulated PTX (positive control) and of saline (negative control), following a single tail vein IV injection (FIG. 16C). In the literature, it is accepted that in vivo tumor growth follows an exponential curve until it reaches a lethal tumor volume of 10¹⁸ cells (1 cubic centimeter).[26] After 10 days post-injection, mice in the saline cohort experienced rapid proliferation until reaching nearly lethal tumor volume within 14 days. By contrast, NP_{L} successfully suppressed tumor growth for up to two weeks post-treatment, and in fact, paralleled that of PTX, within standard error, throughout the duration of the study. This, coupled with the MTD data, suggests that at equivalent doses, enzyme-responsive nanoparticle scaffolds have potentially very low toxicity for equivalent efficacy.

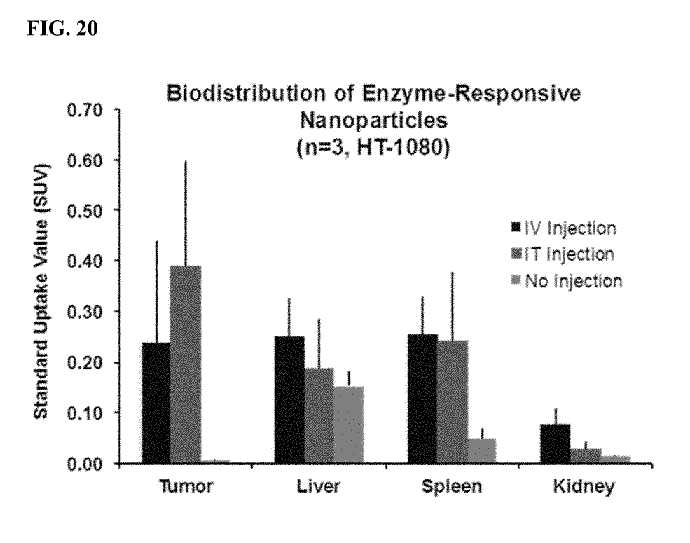

In conjunction with the IV efficacy study, the targeting capabilities of our materials following IV injection were analyzed via live animal fluorescence imaging to monitor for FRET signal generation at the tumor site. Indeed, a FRET signal is generated at the tumor within 3 hours post-injection, and remains observable for up to 3 days. Furthermore, ex vivo tissue analysis of animals sacrificed at 14 days post-injection shows the highest fluorescence signal intensity in the excised tumors, with fluorescence observed to a lesser extent in the liver, spleen, and kidneys (FIG. 17, FIG. 20). This suggests that a mode of clearance of our system is through the reticuloendothelial system (RES). [27] However, the limited toxicity established in the MTD study suggests that although RES-associated organs may sequester these materials, they are not being processed to release their payloads at off-target sites at a rate high enough to achieve toxic doses in the animals. Full pharmacokinetic analysis is underway to further elucidate the underlying mechanisms of these findings.

Together, the foregoing results demonstrate that this novel, innovative class of nanoscale carrier is capable of transporting small molecule chemotherapeutics specifically and selectively to the disease site while limiting off-target toxicity. A distinct advantage of this system is that therapeutic moieties are incorporated into the nanoparticle scaffold via labile covalent bonds enabling high drug-loading, highly reproducible synthesis, and no observable release of material until accumulation occurs at the tumor site. Furthermore, these systems are potentially generalizable, as any therapeutic capable of conjugation to a norbornene handle can be incorporated into the center of the nanoparticle scaffold. Future studies will center on the optimization of this system, and include investigation of higher PTX doses, exploration of the effect of surface chemistry on RES uptake, and tuning the biodegradation of the drug-to-polymer bond via incorporation of linkages sensitive to other stimuli present in tumor tissue, such as lowered pH and oxidative stress. Finally, we note the promising effects observed for IT administration. Although IV administration is certainly the gold standard for development of chemotherapeutics, there are several instances in which IT administration is used clinically, and is highly efficacious against primary and metastatic disease,[28] thus this route may prove a powerful translational tool. In summary, the system introduced here constitutes a new paradigm in the design of drug-carrying nanomaterials: the use of switchable morphology to guide in vivo accumulation for enhanced safety and efficacy.

### Example 4. In Vitro Studies: Additional Materials and Methods

All reagents were obtained from Sigma Aldrich or Fisher Scientific and used without further purification. Polymerizations were performed in a dry, nitrogen atmosphere with anhydrous solvents. MMP-2, -9, and -12 were obtained from Calbiochem as a solution in 200 mM NaCl, 50 mM tris-HCl, 5 mM CaCl₂, 1 uM ZnCl₂, 0.05% BRIJ® 35 Detergent, 0.05% NaN₃, at pH 7.0. HPLC analyses of all products and peptides were performed on a Jupiter 4u Proteo 90A Phenomenex column (150×4.60 mm) with a binary gradient, using a Hitachi-Elite LaChrom 2130 pump that was equipped with a Hitachi-Elite LaChrom L-2420 UV-Vis detector. Separation was achieved with a flow rate of 1 mL min⁻¹ and the following mobile phase: 0.1% trifluoroacetic acid in H₂O (A) and 0.1% trifluoroacetic acid in ACN (B). Starting with 100% A and 0% B, a linear gradient was run for 30 min to a final solvent mixture of 33% A and 67% B, which was held for 5 min before ramping up to 0% A and 100% B over the course of 2 min and holding at this level for an additional 4 minutes, before ramping back down to 100% A and 0% B, with constant holding at this level for 4 additional minutes. Mass spectrometry (MS) of all synthesized compounds and peptides was performed at the Molecular Mass Spectrometry Facility (MMSF) in the Department of Chemistry and Biochemistry at the University of California, San Diego. Polymer dispersities and molecular weights were determined by size-exclusion chromatography (Phenomenex Phenogel 5u 10, 1K-75K, 300×7.80 mm in series with a Phenomenex Phenogel 5u 10, 10K-100K, 300×7.80 mm) in 0.05 M LiBr in DMF, using a Shimatzu pump that was equipped with a multi-angle light scattering detector (DAWN-HELIOS, Wyatt Technology) and a refractive index detectors (Wyatt Optilab T-rEX) normalized to a 30,000 MW polystyrene standard. Hydrodynamic radius (Rh) was determined by DLS, through a Wyatt Dynapro NanoStar. Transmission Electron Microscopy was performed on an FEI Tecnai G2 Sphera at 200 KV. TEM grids were prepared with a 1% uranyl acetate stain on carbon grids from Ted Pella, Inc. In vitro fluorescence measurements were taken on a PTI QuantaMaster Spectrofluorometer. Chemical shifts (¹H) are reported in δ (ppm), relative to the residual proton peak of CDCl₃ (7.27 ppm). Chemical shifts (¹³C) are reported in δ (ppm), relative to the carbon peak of CDCl₃ (77.00 ppm).

Paclitaxel (PTX) Monomer Synthesis:

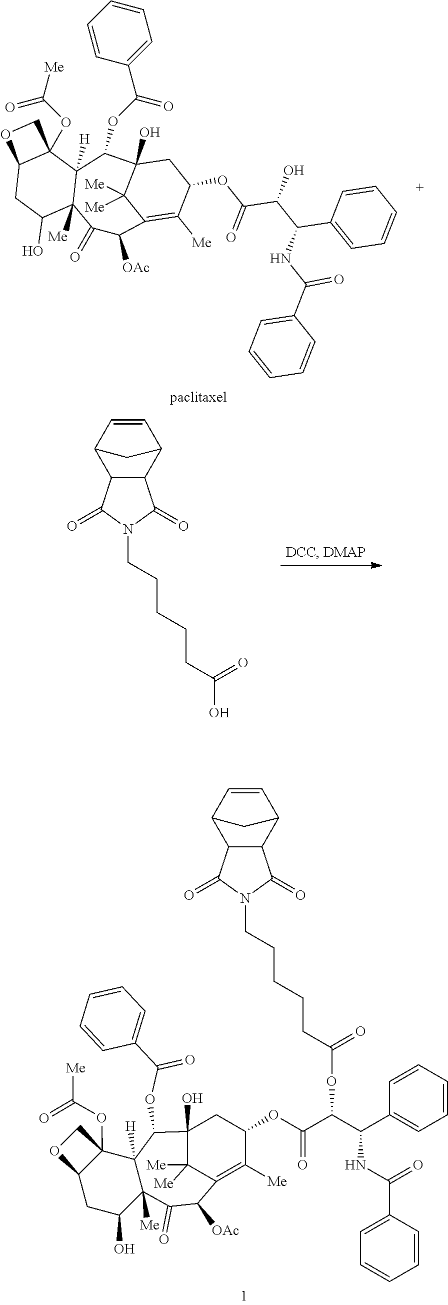 [SMILES: CC(=O)O[C@H]1C(=O)[C@]2(C)C(O)C[C@H]3OC[C@@]3(OC(C)=O)[C@H]2[C@H](OC(=O)c2ccccc2)[C@]2(O)C[C@H](OC(=O)[C@H](O)[C@@H](NC(=O)c3ccccc3)c3ccccc3)C(C)=C1C2(C)C ; O=C(O)CCCCCN1C(=O)C2C3C=CC(C3)C2C1=O ; CC(=O)O[C@H]1C(=O)[C@@]2(C)[C@H]([C@H](OC(=O)c3ccccc3)[C@]3(O)C[C@H](OC(=O)[C@H](OC(=O)CCCCCN4C(=O)C5C6C=CC(C6)C5C4=O)[C@@H](NC(=O)c4ccccc4)c4ccccc4)C(C)=C1C3(C)C)[C@]1(OC(C)=O)CO[C@@H]1C[C@@H]2O]

PTX-Norbornyl Ester, as Depicted Above (1).

To a solution of paclitaxel (2.34×10⁻⁵ mol, 1.0 equiv) with N-(hexanoic acid)-cis-5-norbornene-exo-dicarboximide (2.34×10⁻⁴ mol, 1.0 equiv, prepared from published protocol) in 50 mL dry DMF in a 100 mL round bottom flask under N₂, was added 4-(Dimethylamino)pyridine (2.34×10⁻⁶ mol, 0.1 equiv). After stirring for 5 minutes at 0° C. in an ice bath, N,N′-Dicyclohexylcarbodiimide (2.58×10⁻⁶ mol, 1.1 equiv) was dripped into the reaction mixture and allowed to stir for 7 hours. Reaction progress was monitored via TLC (1:1 hexane: ethyl acetate, R_{f}=0.3). Precipitated urea was removed via suction filtration, and the filter cake washed with DCM. The solvent was removed via rotary evaporation and the resulting crude product was dissolved in 30 mL CHCl₃. Purification was achieved through extraction with water (1×30 mL), followed by 0.5M HCl (3×10 mL), and finally saturated NaHCO₃ (3×10 mL). The organic phase was dried over MgSO₄ and solvent removed via rotary evaporation to afford the purified product in 90% yield.

¹H NMR (400 MHz, CDCl₃): δ (ppm) 1.14-1.33 (m, 8H, 2×CH₃. 1×CH₂) 1.53 (m, 2H, CH₂) 1.58 (m, 2H, CH₂) 1.68 (m, 3H, CH₃) 1.81 (s, 3H, CH₃) 1.88 (m, 2H, CH₂) 1.93 (t, 2H, CH₂) 2.02 (s, 1H, OH) 2.22 (s, 3H, CH₃) 2.34-2.39 (m, 5H, CH₃, CH₂) 2.47 (s, 1H, OH) 3.21 (m, 2H, CH) 3.37-3.48 (m, 2H, CH), 3.80 (d, 1H, CH) 4.19-4.45 (m, 3H, CH₂, CH) 4.96 (t, 1H, CH) 5.51 (d, 1H, CH) 5.68 (d, 1H, CH) 5.98 (t, 1H, CH) 6.22-6.30 (m, 4H, CH) 7.12-7.33 (d, 1H, NH) 7.34-7.75 (m, 18H, 3×Ar). ¹³C NMR (400 MHz, DMSO): 10.2, 14.3, 23.1, 24.3, 26.02, 27.2, 30.83, 32.15, 33.81, 36.23, 38.00, 43.40, 44.91, 47.67, 54.43, 57.83, 70.86, 71.28, 74.95, 77.17, 80.71, 84.07, 125.75, 127.84, 128.77, 129.34, 230.03, 134.64, 137.77, 138.06, 139.82, 154.02, 157.07, 162.74, 165.66, 166.79, 169.18, 170.09, 172.75, 178.03, 202.81. ESI-MS(+): m/z 1136.40 [M+Na]⁺.

Peptide Synthesis:

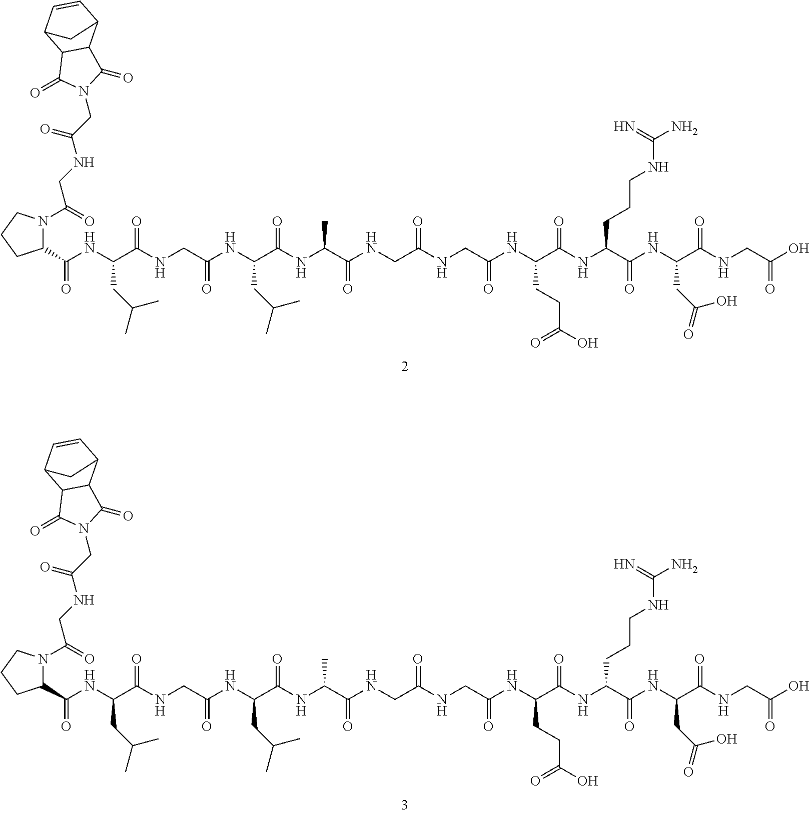

Peptides were synthesized using an AAPPTEC Focus XC automated synthesizer. Both L- and D-amino acids were purchased from AAPPTEC and NovaBiochem. N-(glycine)-cis-5-norbornene-exo-dicarboximide (NorGly) was prepared as described above. Peptide monomers were synthesized via standard FMOC-based peptide synthesis using Rink Amide MBHA resin (AAPTEC) in a standardized fashion. FMOC was deprotected using a solution of 20% 4-methylpiperidine in DMF. Amino acid couplings were carried out using HBTU and DIPEA (resin/amino acid/HBTU/DIPEA 1:3.5:3.4:4). The final peptide monomers were cleaved from the resin using a mixture of TFA/H₂O/TIPS (95:2.5:2.5) for 90 minutes. The peptides were precipitated and washed with cold ether. For purification and analysis, the peptides were dissolved in a solution of 0.1% TFA in water and analyzed via RP-HPLC and purified via preparative HPLC. Peptide identities and purities were confirmed using ESI-MS and RP-HPLC monitoring at I_{abs}=214 nm. Peptide monomer sequence: NorGly-Gly-Pro-Leu-Gly-Leu-Ala-Gly-Gly-Glu-Arg-Asp-Gly. L-amino acids were used exclusively for the preparation of 2, and D-amino acids were used exclusively for the preparation of 3. RP-HPLC retention time was 13 minutes (linear gradient of 0-67% B over 30 minutes). Preparative HPLC retention time was 33 minutes (linear gradient of 20-40% over 60 minutes). ESI-MS(+): 1300.54 [M+H]⁺.

Additional Polymer Synthesis—General Methods: (Compound 1 to Produce Compounds 4L, 4D)

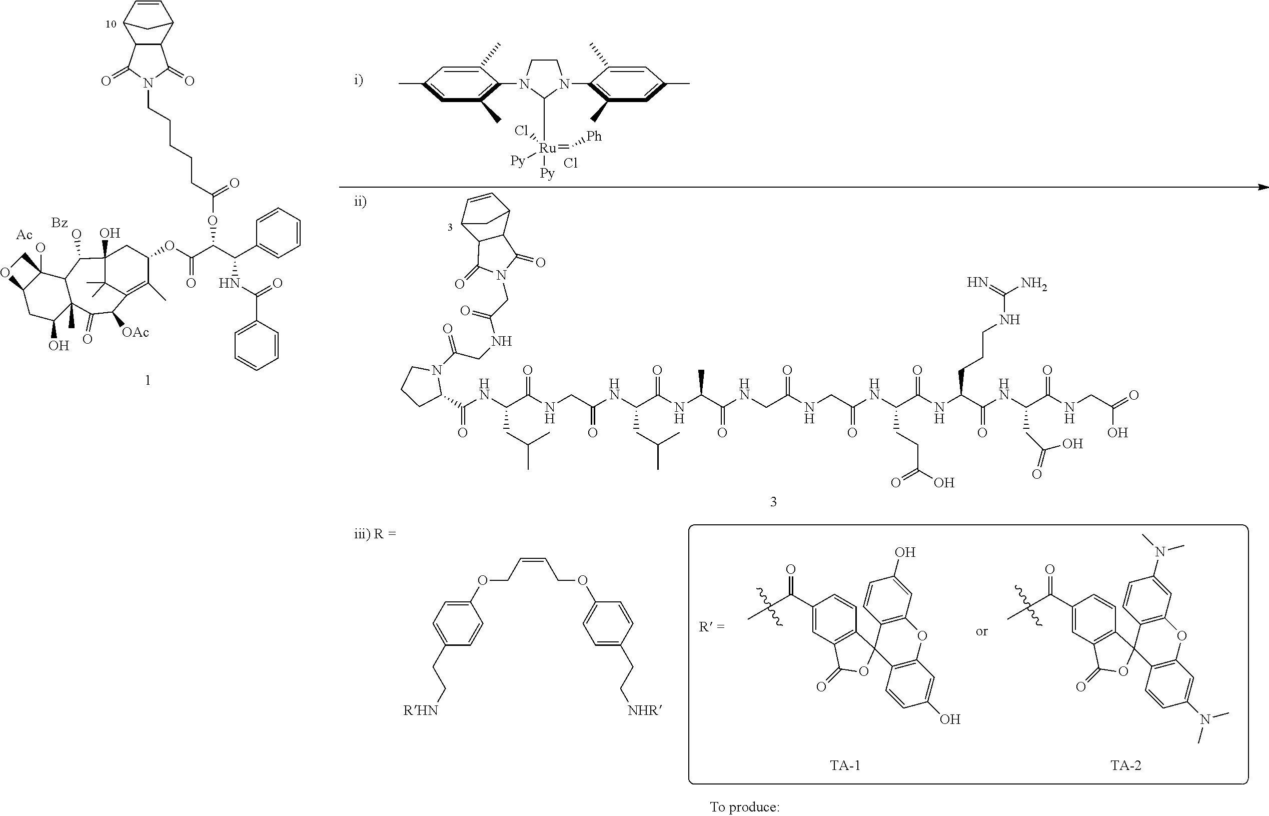

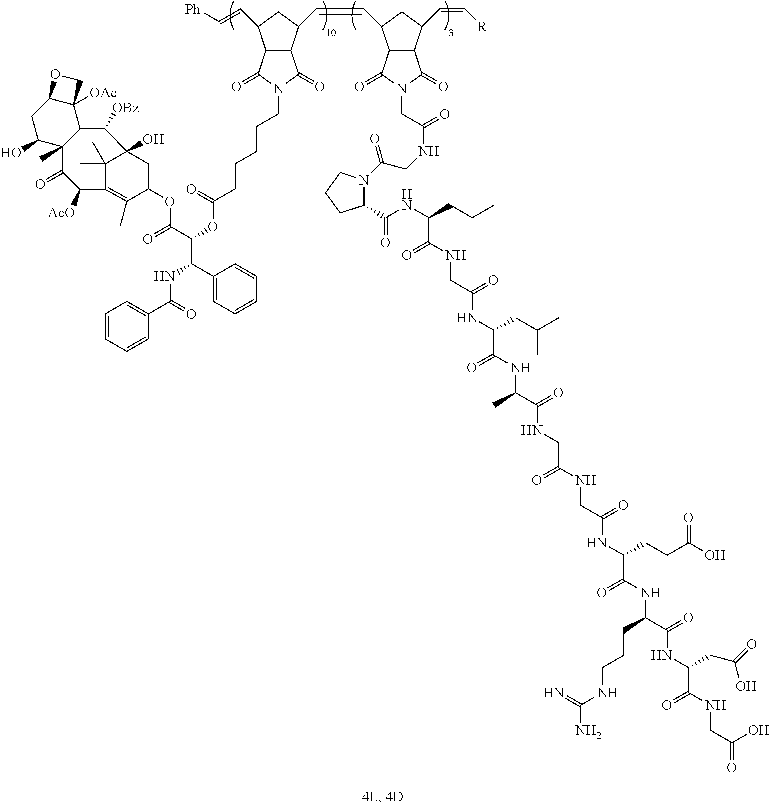

Fluorescein-Terminated Diblock Copolymers (4L, 4D).

To a Stirred Solution of 1 (shown above in the scheme) (143 mg, 1.3×10′ mol, 10 equiv) in dry DMF (1600 μL) was added a solution of the catalyst (shown above in the scheme) ((IMesH₂)(C₅H₅N₂)(Cl)₂Ru═CHPh) (9.22 mg, 1.3×10⁻⁵ mol, 1.0 equiv) in dry DMF (230 μL). The reaction was allowed to stir under N₂ for 2 hours, after which an aliquot (30 μL) was removed and quenched with ethyl vinyl ether for SLS analysis. The remaining solution of 1+catalyst (1800 μL) was split into two separate reaction vessels. To one reaction vessel was added a solution of 2 (50 mg, 3.8×10⁻⁵ mol, 3 equiv) in 800 μL dry DMF (to ultimately afford 4 L). To the second vessel was added a solution of 3 (referred to in step ii in the scheme above) (50 mg, 3.8×10′ mol, 3 equiv) in 800 μL dry DMF (to ultimately afford 4D). After three additional hours, a small aliquot was removed from each reaction vessel (30 μL each) and terminated with ethyl vinyl ether for SLS analysis. To each of the remaining solutions, TA-1 (prepared via previously published protocol) was added (4.5 mg, 6.3×10⁻⁶ mol, 1.2 equiv) and stirred for 1 hour. Afterwards, 10 μL ethyl vinyl ether was added to ensure the polymerizations were fully terminated. 30 μL NH₄OH was then added to both solutions and allowed to stir for an additional 20 minutes to deprotect TA-1. The fully terminated and deprotected polymers were precipitated with a cold 1:1 ether:methanol solution to afford the block copolymers as dark yellow solids (4L, 4D).

Rhodamine-Terminated Diblock Copolymers (5L, 5D).

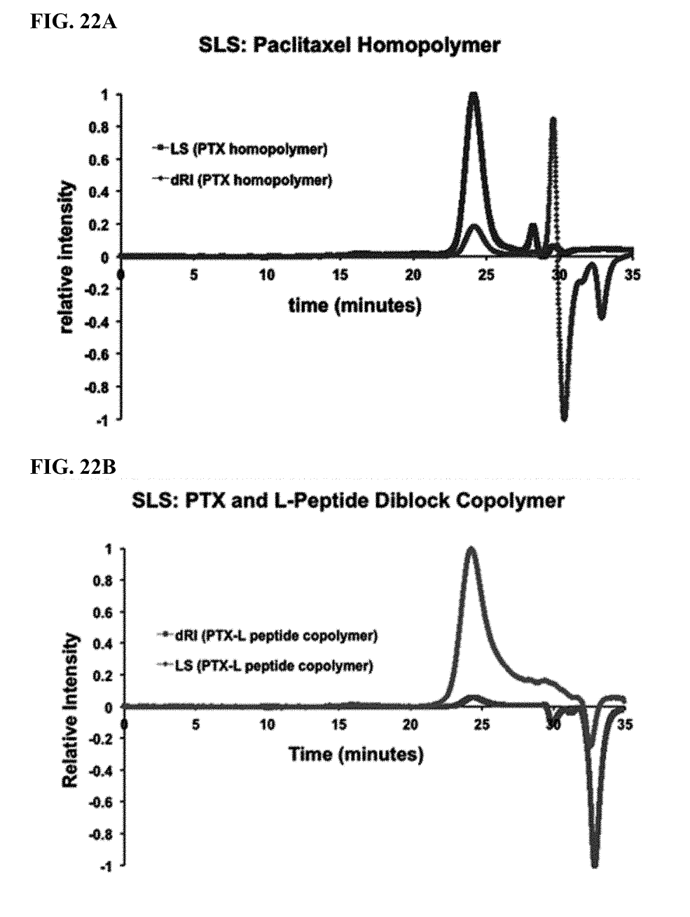

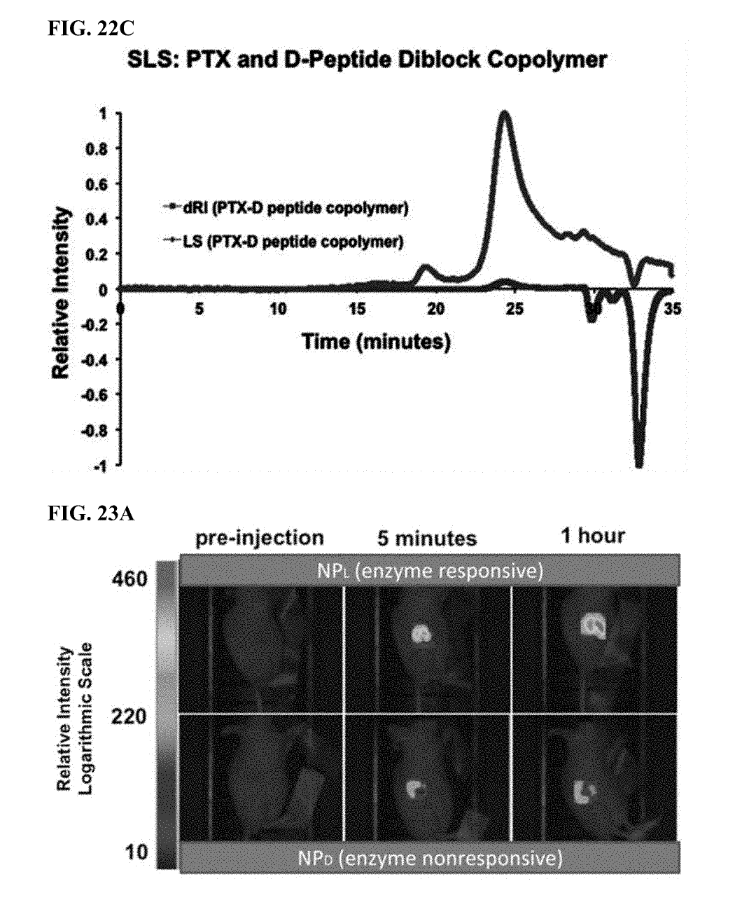

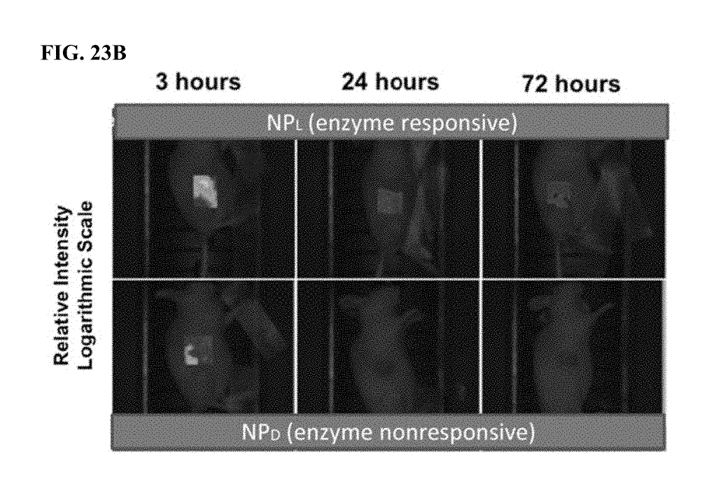

To a stirred solution of 1 (143 mg, 1.3×10⁻⁴ mol, 10 equiv) in dry DMF (1600 μL) was added a solution of the catalyst ((IMesH₂)(C₅H₅N₂)(Cl)₂Ru═CHPh) (9.22 mg, 1.3×10′ mol, 1.0 equiv) in dry DMF (230 μL). The reaction was allowed to stir under N₂ for 2 hours, after which an aliquot (30 μL) was removed and quenched with ethyl vinyl ether for SLS analysis. The remaining solution of 1+catalyst (1800 μL) was split into two separate reaction vessels. To one reaction vessel was added a solution of 2 (50 mg, 3.8×10⁻⁵ mol, 3 equiv) in 800 μL dry DMF (To ultimately afford 5 L). To the second vessel was added a solution of 3 (50 mg, 3.8×10′ mol, 3 equiv) in 800 μL dry DMF (To ultimately afford 5D). After three additional hours, a small aliquot was removed from each reaction vessel (30 μL each) and terminated with ethyl vinyl ether for SLS analysis. To each of the remaining solutions, TA-2 (prepared via previously published protocol) was added (4.5 mg, 6.3×10⁻⁶ mol, 1.2 equiv) and stirred for 1 hour. Afterwards, 10 μL ethyl vinyl ether was added to ensure the polymerizations were fully terminated. The fully terminated polymers were then precipitated with a cold 1:1 ether:methanol solution to afford the block copolymers as deep magenta solids (5L, 5D). See FIGS. 22A-22C for SLS characterization of homo-(A) and diblock-(B,C) copolymers.

Homopolymer of 4L and 4D: Mₙ=13330, PDI=1.016, hydrophobic block length=12. Copolymer of 4 L: Mₙ=17240, PDI=1.035, hydrophilic block length=3. Copolymer of 4D: Mₙ=18830, PDI=1.069, hydrophilic block length=4. Homopolymer of 5L and 5D: Mₙ=8345, PDI=1.024, hydrophobic block length=8. Copolymer of 5L: 10015, PDI=1.027, hydrophilic block length=2. Copolymer of 5D: Mₙ=13030, PDI=1.117, hydrophilic block length=4.

Nanoparticle Preparation (NP_{L} and NP_{D}).

10 mg of polymer was dissolved in 1 mL of DMSO and an additional 1 mL of 1×DPBS (Dulbecco's Phosphate Buffered Saline, no Ca, no Mg) was added over the course of 2 hours. This solution was transferred to a 3500 MWCO snakeskin dialysis tubing, and dialyzed against 1 L of 1×DPBS at pH 7.4 over 2 days with 2 buffer changes. The resulting solution was analyzed by DLS and TEM.

In Vitro Nanoparticle Degradation Via MMP-12.

500 uL NP_{L} (concentration with respect to peptide) NP_{D}, or PTX were incubated with MMP-12 (100 nU) at 37 degrees Celsius. After 4 hours, the assay was quenched by inactivation of MMP-12 at 65° C. for 20 minutes. Aliquots of these quenched samples were removed and analyzed via RP-HPLC (absorbance=214 nmAFIN) to monitor for the presence of peptide cleavage fragment, whose sequence is LAGGERDG (FIG. 18). ESI-MS was conducted on the peak eluted at 14 minutes on the spectrum to determine fragment MW. Only in NP_{L} does this fragment appear, which confirms that enzymatic degradation of the peptide shell will only occur if the sequence is composed of L-amino acids. Analogous experiments were run using MMP-2 and MMP-9, whose results are the same. The elution time for paclitaxel is 26 minutes, confirmed by both HPLC of PTX incubated with MMP under the same conditions and by ESI-MS (FIG. 21). Samples were also analyzed by DLS and negative stain TEM to monitor for aggregation following MMP cleavage. This aggregation event only occurs in the NP_{L} system, as evidenced by TEM and DLS (FIG. 19).

In Vivo Studies.

Paclitaxel Injection USP (Hospira, Inc.) was graciously donated by UCSD Moores Cancer Center (3855 Health Sciences Drive, La Jolla, Calif.). Tumors grown from HT-1080 fibrosarcoma cells (ATCC) were used for the model system, as this cell line overexpress MMPs. Nu/nu mice were obtained through the UCSD in-house colony. Animals were inoculated with ˜10⁶ cells as a subcutaneous bolus, and treatments began once tumor mass reached ˜50 mm³. Animals were sacrificed at 14 days post-treatment, or when their tumor burdens exceeded 1500 mm³. B-mode ultrasound (US) (Visualsonics Inc, Vevo 770-120) was used to record tumor volume daily over the course of the study. Absolute tumor volume was approximated with the formula V=length (mm)×width (mm)×depth (mm), as determined from US images. Relative tumor volume was determined by the formula: Vᵣₑₗₐₜᵢᵥₑ=(v/vᵢ)*100.

In the above formula v is the absolute tumor volume on the day of measurement and vᵢ is the absolute tumor volume on the first day of treatment. Live-animal imaging was taken on a GE Art Optix instrument.

For optical imaging, animals were anesthetized with isofluorane with an induction dose of 3% and a maintenance dose of 1.5% in an oxygen gas stream. After injection, animals were imaged at given timepoints using a GE ART eXplore Optix Instrument (λₑₓ=470 nm and λₑₘ=590 nm). Animals were sacrificed after experiments. Organs (liver, spleen, lung, kidney, heart, and lung) and tumor were harvested and frozen for tissue slice preparation and analysis. To examine chronic in vivo toxicity, histological examination was conducted on sections of liver and kidney, 14 days post-IV injection of NP_{L} and NP_{D}. Organs were removed and frozen using cryoprotection and OCT. The tissue was then sectioned with a cryostat at 5 μm thickness, and stained with haemoatoxylin and eosin.

Maximum Tolerated Dose (MTD) of NP_{L}.

15 healthy nu/nu female mice were separated into 5 groups (3 mice per cohort) and treated with NP_{L} at the dosage equivalent of 15, 30, 60, 120, or 240 mg/kg with respect to PTX as a single, tail vein IV injection. Mouse weight was recorded once daily until all animals in the cohort returned to, or surpassed, their weight on the day of injection. Adverse toxicity is measured as a function of lethality and/or weight loss, with >20% weight loss suggestive of severe adverse events. No animals in any cohort given NP_{L} died. Two additional cohorts (6 mice, 3 per group) were treated with 15 mg/kg and 30 mg/kg of clinically formulated paclitaxel (a 6 mg/mL suspension in 1:1 ethanol:Cremophor EL®, diluted with 1×DPBS prior to injection). Animals in the 15 mg/kg PTX cohort experienced similar weight-loss as those in the 15 mg/kg PTX-NP cohort (see FIG. 16A), but with serious reactions as a result of the method of injection (tails necrosed below the injection site and subsequently fell off). In the 30 mg/kg group, 1 out of the 3 animals died within 30 minutes post-injection. An additional animal was given 30 mg/kg and also died within an hour of injection.

Intravenous Efficacy.

20 tumor-bearing nu/nu female mice were randomly sorted into 4 groups (5 mice per cohort) and treated with NP_{L}, NP_{D}, PTX, or saline at the dosage equivalent of 15 mg/kg of PTX as a single, tail-vein IV injection. Mouse weight and tumor volume were recorded once daily over the course of the 14-day study. Animals were imaged at 0, 1, 3, 5, 24, 48, 72, and 126 hours post-injection via live-animal optical imaging. To assess efficacy, relative tumor volume (see equation (1) above) was calculated for each data point. The average relative tumor volume of each cohort at each time point was then calculated, along with standard deviation and standard error. Animals were sacrificed at 14 days post-injection. Tumor, liver, spleen, kidneys, heart, and lungs were excised from each animal and treated as in above protocol.

Intratumoral Efficacy.

15 tumor-bearing nu/nu female mice were randomly sorted into 3 groups (5 mice per cohort) and treated with NP_{L}, NP_{D}, or saline at the dosage equivalent of 15 mg/kg of PTX as a single intratumoral injection. Mouse weight and tumor volume were recorded once daily over the course of the 12-day study. Animals were imaged at 0, 1, 3, 24, 48, and 72 hours post-injection via live-animal optical imaging. To assess efficacy, relative tumor volume (see equation above) was calculated for each data point. The average relative tumor volume of each cohort at each time point was then calculated, along with standard deviation and standard error. Animals were sacrificed at 12 days post-injection. Tumor, liver, spleen, kidneys, heart, and lungs were excised from each animal and treated as in above protocol. An additional tumor-bearing mouse was administered 15 mg/kg of PTX, also as a single intratumoral injection. Following treatment, the animal experienced severe ulceration at the tumor site within two days of treatment, and thus had to be sacrificed and excluded from the study.

Biodistribution by Standardized Uptake Values.

6 tumor-bearing nu/nu female mice were sorted randomly into 2 groups (3 mice per cohort) and treated with NP_{L} at the dosage equivalent of 15 mg/kg of PTX, as either an IT or IV injection. An additional cohort of 3 tumor-bearing mice was given no treatment, to serve as a handle for baseline tissue fluorescence. At 48 hours post-injection, all animals were sacrificed. Tumor, liver, spleen, and kidneys of all animals were harvested, weighed, and transferred to individual 15 mL conical tubes. Lysis buffer (0.25 mg/mL Proteinase K, 0.1 mg/mL DNAse, 150 mM NaCl, 10 mM tris pH 8, 0.2% SDS) was added to each conical tube as a ratio of 9 μL buffer per 1 mg tissue. All tissues were then cut into small pieces and homogenized for 30 seconds using an ultrasonicator, transferred to 1.5 mL eppendorf tubes, and incubated overnight at 55° C.

Calibration curves for each tissue type (liver, spleen, kidney, tumor) were generated via the following protocol: 90 μL of homogenated tissue from animals treated with saline were pipetted into a 96-well plate and fluorescence measured to obtain background. 10 μL of NP_{L} was added at varying polymer concentrations and the fluorescence measured (λ_{excitation}=545 nm, λ_{excitation}=580 nm), to generate a plot of fluorescence counts vs. polymer concentration. The data obtained from each curve was fitted linearly to use for measuring polymer concentration in experimental tissues. The SUV calibration curve for the tumor provided a linear fit equation with an R² of 0.9595, and the equation: y=4×10⁹x−159.31. The SUV calibration curve for the liver provided a linear fit equation with an R² of 0.93846, and the equation: y=5×10⁹x−1545.5. The SUV calibration curve for the spleen provided a linear fit equation with an R² of 0.98514, and the equation: y=3×10⁹x−495.19. The SUV calibration curve for the kidney provided a linear fit equation with an R² of 0.97125, and the equation: y=4×10⁹x−128.59.

To assess the effect of injection method on the biodistribution of NP_{L}, the fluorescence count of NP_{L} in tumor, liver, spleen, and kidneys after either IT or IV injection was measured. 100 μL of each homogenated tissue (see details above) was added to an individual well of a 96-well plate. Fluorescent counts of each well λ_{excitation}=545 nm, λ_{excitation}=580 nm) were converted to polymer concentrations using tissue-specific calibration curves generated as described above, and were then normalized by the injected dose and animal weight to calculate standardized uptake values (SUV=(moles of polymer in tissue/mass of tissue)/(mols of polymer injected/weight of animal).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

### References from Example 1

[1] D. Mozaffarian, E. J. Benjamin, A. S. Go, D. K. Arnett, M. J. Blaha, M. Cushman, S. de Ferranti, J. P. Despres, H. J. Fullerton, V. J. Howard, M. D. Huffman, S. E. Judd, B. M. Kissela, D. T. Lackland, J. H. Lichtman, L. D. Lisabeth, S. Liu, R. H. Mackey, D. B. Matchar, D. K. McGuire, E. R. Mohler, 3rd, C. S. Moy, P. Muntner, M. E. Mussolino, K. Nasir, R. W. Neumar, G. Nichol, L. Palaniappan, D. K. Pandey, M. J. Reeves, C. J. Rodriguez, P. D. Sorlie, J. Stein, A. Towfighi, T. N. Turan, S. S. Virani, J. Z. Willey, D. Woo, R. W. Yeh, M. B. Turner, C. American Heart Association Statistics, S. Stroke Statistics, Circulation 2015, 131, e29; [2] M. Dobaczewski, C. Gonzalez-Quesada, N. G. Frangogiannis, in J. Mol. Cell. Cardiol., Vol. 48, 2010, 504; [3] N. G. Frangogiannis, C. W. Smith, M. L. Entman, Cardiovascular research 2002, 53, 31; [4] Y. Sun, K. T. Weber, Cardiovasc Res 2000, 46, 250; D. L. Mann, Circulation 1999, 100, 999; F. Yang, Y. H. Liu, X. P. Yang, J. Xu, A. Kapke, O. A. Carretero, Experimental physiology 2002, 87, 547; [5] A. A. Rane, K. L. Christman, J Am Coll Cardiol 2011, 58, 2615; [6] E. Tous, B. Purcell, J. L. Ifkovits, J. A. Burdick, Journal of cardiovascular translational research 2011, 4, 528; D. M. Nelson, Z. W. Ma, K. L. Fujimoto, R. Hashizume, W. R. Wagner, Acta Biomaterialia 2011, 7, 1; [7] T. D. Johnson, K. L. Christman, Expert Opin Drug Del 2013, 10, 59; [8] J. L. Ungerleider, K. L. Christman, Stem cells translational medicine 2014, 3, 1090; [9] L. D. Horwitz, D. Kaufman, M. W. Keller, Y. O. Kong, Circulation 1994, 90, 2439; [10] F. Alexis, E. Pridgen, L. K. Molnar, O. C. Farokhzad, in Mol. Pharmaceutics, Vol. 5, 2008, 505; [11] S. Suarez, A. Almutairi, K. L. Christman, Biomaterials Science 2015, 3, 564; [12] L. E. Paulis, T. Geelen, M. T. Kuhlmann, B. F. Coolen, M. Schaefers, K. Nicolay, G. J. Strijkers, Journal of Controlled Release 2012, 162, 276; M.-Y. Chang, Y.-J. Yang, C.-H. Chang, A. C. L. Tang, W.-Y. Liao, F.-Y. Cheng, C.-S. Yeh, J. J. Lai, P. S. Stayton, P. C. H. Hsieh, J. Control Release 2013, 170, 287; [13] T. Dvir, M. Bauer, A. Schroeder, J. H. Tsui, D. G. Anderson, R. Langer, R. Liao, D. S. Kohane, Nano Letters 2011, 11, 4411; [14] Y. W. Won, A. N. McGinn, M. Lee, D. A. Bull, S. W. Kim, Molecular Pharmaceutics 2013, 10, 378; S. Kanki, D. E. Jaalouk, S. Lee, A. Y. Yu, J. Gannon, R. T. Lee, Journal of molecular and cellular cardiology 2011, 50, 841; [15] M.-P. Chien, M. P. Thompson, C. V. Barback, T.-H. Ku, D. J. Hall, N. C. Gianneschi, in Adv. Mater. Weinheim, Vol. 25, 2013, 3599; [16] M.-P. Chien, M. P. Thompson, C. V. Barback, T.-H. Ku, D. J. Hall, N. C. Gianneschi, Advanced Materials 2013, 25, 3599; [17] F. G. Spinale, in Physiol. Rev., Vol. 87, American Physiological Society, 2007, 1285; [18] D. M. Nelson, Z. Ma, K. L. Fujimoto, R. Hashizume, W. R. Wagner, Acta Biomaterialia 2011, 7, 1; [19] A. Galaup, E. Gomez, R. Souktani, M. Durand, A. Cazes, C. Monnot, J. Teillon, S. Le Jan, C. Bouleti, G. Briois, J. Philippe, S. Pons, V. Martin, R. Assaly, P. Bonnin, P. Ratajczak, A. Janin, G. Thurston, D. M. Valenzuela, A. J. Murphy, G. D. Yancopoulos, R. Tissier, A. Berdeaux, B. Ghaleh, S. Germain, Circulation 2012, 125, 140; [20] A. N. Lukyanov, W. C. Hartner, V. P. Torchilin, Journal of Controlled Release 2004, 94, 187; [21] T. Geelen, L. E. Paulis, B. F. Coolen, K. Nicolay, G. J. Strijkers, in Contrast Media Mol Imaging, Vol. 8, 2013, 117; [22] R. C. Scott, J. M. Rosano, Z. Ivanov, B. Wang, P. L.-G. Chong, A. C. Issekutz, D. L. Crabbe, M. F. Kiani, in FASEB J., Vol. 23, 2009, 3361; [23] S. Mizrahy, M. Goldsmith, S. Leviatan-Ben-Arye, E. Kisin-Finfer, O. Redy, S. Srinivasan, D. Shabat, B. Godin, D. Peer, Nanoscale 2014, 6, 3742; Y. Zhong, F. Meng, C. Deng, Z. Zhong, Biomacromolecules 2014, 15, 1955; [24] C. Zhang, C. Li, Y. Liu, J. Zhang, C. Bao, S. Liang, Q. Wang, Y. Yang, H. Fu, K. Wang, D. Cui, Adv. Funct. Mater. 2015, 25, 1314; M. Y. Chang, Y. J. Yang, C. H. Chang, A. C. L. Tang, W. Y. Liao, F. Y. Cheng, C. S. Yeh, J. J. Lai, P. S. Stayton, P. C. H. Hsieh, J Control Release 2013, 170, 287; [25] J. Nguyen, R. Sievers, J. P. Motion, S. Kivimae, Q. Fang, R. J. Lee, Molecular pharmaceutics 2015, 12, 1150; [26] J. M. Lambert, E. F. Lopez, M. L. Lindsey, Int J Cardiol 2008, 130, 147; [27] L. E. Paulis, T. Geelen, M. T. Kuhlmann, B. F. Coolen, M. Schafers, K. Nicolay, G. J. Strijkers, Journal of Controlled Release 2012, 162, 276; [28] W.-S. Cho, M. Cho, J. Jeong, M. Choi, H.-Y. Cho, B. S. Han, S. H. Kim, H. O. Kim, Y. T. Lim, B. H. Chung, J. Jeong, Toxicology and Applied Pharmacology 2009, 236, 16; [29] H. J. Johnston, G. Hutchison, F. M. Christensen, S. Peters, S. Hankin, V. Stone, Critical Reviews in Toxicology 2010, 40, 328; [30] D. P. K. Lankveld, A. G. Oomen, P. Krystek, A. Neigh, A. Troost-de Jong, C. W. Noorlander, J. C. H. Van Eijkeren, R. E. Geertsma, W. H. De Jong, Biomaterials 2010, 31, 8350; G. Sonavane, K. Tomoda, K. Makino, Colloid Surface B 2008, 66, 274; [31] H. C. Fischer, W. C. W. Chan, Current Opinion in Biotechnology 2007, 18, 565.

### References from Example 2

[1] T.-H. Ku, M.-P. Chien, M. P. Thompson, R. S. Sinkovits, N. H. Olson, T. S. Baker, N. C. Gianneschi, Journal of the American Chemical Society 2011, 133, 8392; [2] J. K. Pontrello, M. J. Allen, E. S. Underbakke, L. L. Kiessling, Journal of the American Chemical Society 2005, 127, 14536; [3] M. P. Thompson, L. M. Randolph, C. R. James, A. N. Davalos, M. E. Hahn, N. C. Gianneschi, Polymer Chemistry 2014, 5, 1954; [4] M. S. Sanford, J. A. Love, R. H. Grubbs, Organometallics 2001, 20, 5314; [5] S. B. Seif-Naraghi, D. Horn, P. J. Schup-Magoffin, K. L. Christman, Acta Biomaterialia 2012, 8, 3695; [6] T. D. Johnson, R. L. Braden, K. L. Christman, Methods in molecular biology 2014, 1181, 109; [7] J. M. Singelyn, P. Sundaramurthy, T. D. Johnson, P. J. Schup-Magoffin, D. P. Hu, D. M. Faulk, J. Wang, K. M. Mayle, K. Bartels, M. Salvatore, A. M. Kinsey, A. N. DeMaria, N. Dib, K. L. Christman, J Am Coll Cardiol 2012, 59, 751; [8] C. W. Bielawski, R. H. Grubbs, Progress in Polymer Science 2007, 32, 1.

### References from Example 3

[1] a) M. E. Davis, Z. Chen, D. M. Shin, Nat. Rev. Drug Discov. 2008, 7, 771; b) A. P. Blum, J. K. Kammeyer, A. M. Rush, C. E. Callmann, M. E. Hahn, N. C. Gianneschi, J. Am. Chem. Soc. 2015, 137, 2140. [2] a) Y. H. Bae, K. Park, J. Controlled Release 2011, 153, 198; b) H. Kobayashi, R. Watanabe, P. L. Choyke, Theranostics 2014, 4, 81; c) R. K. Jain, T. Stylianopoulos, Nat. Rev. Clin. Oncol. 2010, 7, 653. [3] H. Maeda, J. Wu, T. Sawa, Y. Matsumura, K. Hori, J. Controlled Release 2000, 65, 271. [4] F. Danhier, O. Feron, V. Préat, J J. Controlled Release 2010, 148, 135. [5] a) W. Arap, R. Pasqualini, E. Ruoslahti, Science 1998, 279, 377; b) M. A. Horton, Int. J. Biochem. Cell Biol. 1997, 29, 721; c) H. Jin, J. Varner, Br. J. Cancer 2004, 90, 561; d) K. Temming, R. M. Schiffelers, G. Molema, R. J. Kok, Drug Resist. Updates 2005, 8, 381; e) M. D. Pierschbacher, E. Ruoslahti, Nature 1984, 309, 30; f) J. Lawler, R. Weinstein, R. O. Hynes, J. Cell Biol. 1988, 107, 2351. [6] a) R. Pasqualini, E. Koivunen, R. Kain, J. Landenranta, M. Sakamoto, A. Stryhn, R. A. Ashmun, L. H. Shapiro, W. Arap, E. Ruoslahti, Cancer Res. 2000, 60, 722; b) F. Curnis, A. Sacchi, L. Borgna, F. Magni, A. Gasparri, A. Corti, Nat. Biotech. 2000, 18, 1185; c) S. V. Garde, A. J. Forté, M. Ge, E. A. Lepekhin, C. J. Panchal, S. A. Rabbani, J. J. Wu, Anti-Cancer Drugs 2007, 18, 1189; d) F. Pastorino, C. Brignole, D. Marimpietri, M. Cilli, C. Gambini, D. Ribatti, R. Longhi, T. M. Allen, A. Corti, M. Ponzoni, Cancer Res. 2003, 63, 7400. [7] a) P. S. Low, A. C. Antony, Adv. Drug Delivery Rev. 2004, 56, 1055; b) B. Stella, S. Arpicco, M. T. Peracchia, D. Desmaële, J. Hoebeke, M. Renoir, J. D'Angelo, L. Cattel, P. Couvreur, J Pharm. Sci. 2000, 89, 1452; c) C. P. Leamon, J. A. Reddy, Adv. Drug Delivery Rev. 2004, 56, 1127; d) R. J. Lee, P. S. Low, Biochim. Biophys. Acta, Biomembr. 1995, 1233, 134; e) C. Leamon, P. Low, J. Drug Targeting 1994, 2, 101. [8] a) A. K. Galande, S. A. Hilderbrand, R. Weissleder, C.-H. Tung, J. Med. Chem. 2006, 49, 4715; b) Q. T. Nguyen, E. S. Olson, T. A. Aguilera, T. Jiang, M. Scadeng, L. G. Ellies, R. Y. Tsien, Proc. Nat. Ac. Soc. 2010, 107, 4317. [9] M.-P. Chien, M. P. Thompson, E. C. Lin, N. C. Gianneschi, Chem. Sci. 2012, 3, 2690. [10] M.-P. Chien, M. P. Thompson, C. V. Barback, T.-H. Ku, D. J. Hall, N. C. Gianneschi, Adv. Mat 2013, 25, 3599. [11] M.-P. Chien, A. S. Carlini, D. Hu, C. V. Barback, A. M. Rush, D. J. Hall, G. Orr, N. C. Gianneschi, J. Am. Chem. Soc. 2013, 135, 18710. [12] a) K. Kessenbrock, V. Plaks, Z. Werb, Cell 2010, 141, 52; E. S. Olson, T. Jiang, T. A. Aguilera, Q. T. Nguyen, L. G. Ellies, M. Scadeng, R. Y. Tsien, Proc. Nat. Ac. Soc. 2010, 107, 4311; b) C. Bremer, S. Bredow, U. Mahmood, R. Weissleder, C.-H. Tung, Radiology 2001, 221, 523; c) T. Jiang, E. S. Olson, Q. T. Nguyen, M. Roy, P. A. Jennings, R. Y. Tsien, Proc. Nat. Ac. Soc. 2004, 101, 17867. [13] C. Metildi, C. Felsen, E. Savariar, Q. Nguyen, S. Kaushal, R. Hoffman, R. Tsien, M. Bouvet, Ann. Surg. Oncol. 2014, 1. [14] a) C. W. Bielawski, R. H. Grubbs, Angew. Chem., Int. Ed. 2000, 39, 2903; b) T. M. Trnka, R. H. Grubbs, Acc. Chem. Res. 2001, 34, 18; c) C. Slugovc, Macromol. Rapid Commun. 2004, 25, 1283; d) M. S. Sanford, J. A. Love, R. H. Grubbs, Organometallics 2001, 20, 5314; e) M. S. Sanford, J. A. Love, R. H. Grubbs, J. Am. Chem. Soc. 2001, 123, 6543; f) R. H. Grubbs, Tetrahedron 2004, 60, 7117. [15] M. Scholl, S. Ding, C. W. Lee, R. H. Grubbs, Org. Lett. 1999, 1, 953. [16] J. K. Kammeyer, A. P. Blum, L. Adamiak, M. E. Hahn, N. C. Gianneschi, Polym. Chem. 2013, 4, 3929. [17] a) S. C. G. Biagini, R. Gareth Davies, M. North, V. C. Gibson, M. R. Giles, E. L. Marshall, D. A. Robson, Chem. Comm. 1999, 235; b) H. D. Maynard, S. Y. Okada, R. H. Grubbs, Macromolecules 2000, 33, 6239. [18] a) A. Leitgeb, J. Wappel, C. Slugovc, Polymer 2010, 51, 2927; b) S. Sutthasupa, M. Shiotsuki, F. Sanda, Polym. J. 2010, 42, 905. [19] a) P. B. Schiff, J. Fant, S. B. Horwitz, Nature 1979, 277, 665; b) S. Band Horwitz, Trends Pharmacol. Sci. 1992, 13, 134. [20] E. K. Rowinsky, R. C. Donehower, N Engl. J. Med. 1995, 332, 1004. [21] A. P. Blum, J. K. Kammeyer, J. Yin, D. T. Crystal, A. M. Rush, M. K. Gilson, N. C. Gianneschi, J. Am. Chem. Soc. 2014, 136, 15422. [22] S.-O. Yoon, M.-M. Kim, A.-S. Chung, J Biol. Chem. 2001, 276, 20085. [23] a) S. Cao, J. D. Black, A. B. Troutt, Y. M. Rustum, Cancer Res. 1998, 58, 3270; b) S. Cao, Y. M. Rustum, Cancer Res. 2000, 60, 3717. [24] a) G. S. Huang, L. Lopez-Barcons, B. S. Freeze, A. B. Smith, G. L. Goldberg, S. B. Horwitz, H. M. McDaid, Clin. Cancer Res. 2006, 12, 298; b) T. Yamori, S. Sato, H. Chikazawa, T. Kadota, Jpn. J Cancer Res. 1997, 88, 1205; c) P. A. Trail, D. Willner, A. B. Bianchi, A. J. Henderson, M. D. TrailSmith, E. Girit, S. Lasch, I. Hellström, K. E. Hellström, Clin. Cancer Res. 1999, 5, 3632. [25] G. D. Ayers, E. T. McKinley, P. Zhao, J. M. Fritz, R. E. Metry, B. C. Deal, K. M. Adlerz, R. J. Coffey, H. C. Manning, J. U. M. 2010, 29, 891. [26] a) L. Simpson-Herren, H. H. Lloyd, Cancer Chemother. Rep., Part 1 1970, 54, 143; b) E. D. Yorke, Z. Fuks, L. Norton, W. Whitmore, C. C. Ling, Cancer Res. 1993, 53, 2987. [27] R. Gref, A. Domb, P. Quellec, T. Blunk, R. H. Müller, J. M. Verbavatz, R. Langer, Adv. Drug Delivery Rev. 1995, 16, 215.[28] a) E. P. Goldberg, A. R. Hadba, B. A. Almond, J. S. Marotta, J Pharm. Pharmacol. 2002, 54, 159; b) K. A. Walter, R. J. Tamargo, A. Olivi, P. C. Burger, H. Brem, Neurosurgery 1995, 37, 1129; c) Y. Tong, W. Song, R. G. Crystal, Cancer Res. 2001, 61, 7530; d) T. Lammers, P. Peschke, R. Kühnlein, V. Subr, K. Ulbrich, P. Huber, W. Hennink, G. Stormy, NEOPFL 2006, 8, 788; e) F. Celikoglu, S. I. Celikoglu, E. P. Goldberg, Lung Cancer 2008, 61, 1.

### EMBODIMENTS

Terms defined herein refer only to aspects and embodiments within this section.

In another aspect, there is provided a nanoparticle including a peptide-polymer amphiphile (PPA) as disclosed herein, wherein the nanoparticle undergoes a morphology switch from nanoparticle to aggregate assembly in response to contact with infracted myocardium.

In another aspect, there is provided a polymer including an enzymatic cleavable moiety, wherein the enzyme cleavable moiety is cleaved by an enzyme unregulated in heart tissue post-myocardial infarction.

In another aspect, there is provided a method for treating myocardial infarction in a subject. The method includes administering to a subject in need thereof a nanoparticle as disclosed herein, wherein the administering is via intravenous, intracoronary, or intramyocardial delivery.

In another aspect, there is provided a method for treating myocardial infarction in a subject. The method includes administering to a subject in need thereof a polymer as disclosed herein, wherein the administering is via intravenous, intracoronary, or intramyocardial delivery.

In another aspect, there is provided a pharmaceutical composition including a nanoparticle composition, a nanoparticle, or a polymer as disclosed herein in combination with a pharmaceutically acceptable excipient.

In another aspect, there is provided a nanoparticle composition including a responsive nanoparticle. The responsive nanoparticle includes an enzyme responsive peptide-polymer amphiphile (PPA) and an enzymatically cleavable moiety, wherein the enzymatically cleavable moiety is cleaved by an enzyme which is upregulated in heart tissue post-myocardial infarction.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀, means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, B, As, and Si may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heteroalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A “fused ring aryl-heterocycloalkyl” is an aryl fused to a heterocycloalkyl. A “fused ring heteroaryl-heterocycloalkyl” is a heteroaryl fused to a heterocycloalkyl. A “fused ring heterocycloalkyl-cycloalkyl” is a heterocycloalkyl fused to a cycloalkyl. A “fused ring heterocycloalkyl-heterocycloalkyl” is a heterocycloalkyl fused to another heterocycloalkyl. Fused ring aryl-heterocycloalkyl, fused ring heteroaryl-heterocycloalkyl, fused ring heterocycloalkyl-cycloalkyl, or fused ring heterocycloalkyl-heterocycloalkyl may each independently be unsubstituted or substituted with one or more of the substituents described herein. Fused ring aryl-heterocycloalkyl, fused ring heteroaryl-heterocycloalkyl, fused ring heterocycloalkyl-cycloalkyl, or fused ring heterocycloalkyl-heterocycloalkyl may each independently be named according to the size of each of the fused rings. Thus, for example, 6.5 aryl-heterocycloalkyl fused ring describes a 6 membered aryl moiety fused to a 5 membered heterocycloalkyl.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The term “oxo,” as used herein, means an oxygen that is double bonded to a carbon atom.

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R, —S(O)₂R′, —S(O)₂NR′R″, —NRSO₂R′, —NR′NR″R′″, —ONR′R″, —NR′C(O)NR″NR′″R″, —CN, —NO₂, —NR′SO₂R″, —NRC(O)R″, —NRC(O)—OR″, —NR′OR″, in a number ranging from zero to (2m′+1), where m′ is the total number of carbon atoms in such radical. R, R′, R″, R′″, and R″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SW, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R″′)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one or more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. The ring-forming substituents may be attached to adjacent members of the base structure. For example, two ring-forming substituents attached to adjacent members of a cyclic base structure create a fused ring structure. The ring-forming substituents may be attached to a single member of the base structure. For example, two ring-forming substituents attached to a single member of a cyclic base structure create a spirocyclic structure. The ring-forming substituents may be attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), Boron (B), Arsenic (As), and silicon (Si).

A “substituent group,” as used herein, means a group selected from the following moieties:
(A) oxo, halogen, —CF₃, —CN, —OH, —NH₂, —COOH, —CONH₂, —NO₂, —SH, SO₂Cl, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, halogen, —CF₃, —CN, —OH, —NH₂, —COOH, —CONH₂, —NO₂, —SH, SO₂Cl, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, halogen, —CF₃, —CN, —OH, —NH₂, —COOH, —CONH₂, —NO₂, —SH, SO₂Cl, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, halogen, —CF₃, —CN, —OH, —NH₂, —COOH, —CONH₂, —NO₂, —SH, SO₂Cl, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and each substituted or unsubstituted heterocycloalkyl is a substituted or unsubstituted 3 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl.

Each substituted group described in the compounds herein may be substituted with at least one substituent group. More specifically, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein may be substituted with at least one substituent group. At least one or all of these groups may be substituted with at least one size-limited substituent group. At least one or all of these groups may be substituted with at least one lower substituent group.

Each substituted or unsubstituted alkyl may be a substituted or unsubstituted C₁-C₂₀ alkyl, each substituted or unsubstituted heteroalkyl may be a substituted or unsubstituted 2 to 20 membered heteroalkyl, each substituted or unsubstituted cycloalkyl may be a substituted or unsubstituted C₃-C₈ cycloalkyl, and/or each substituted or unsubstituted heterocycloalkyl may be a substituted or unsubstituted 3 to 8 membered heterocycloalkyl. Each substituted or unsubstituted alkylene may be a substituted or unsubstituted C₁-C₂₀ alkylene, each substituted or unsubstituted heteroalkylene may be a substituted or unsubstituted 2 to 20 membered heteroalkylene, each substituted or unsubstituted cycloalkylene may be a substituted or unsubstituted C₃-C₈ cycloalkylene, and/or each substituted or unsubstituted heterocycloalkylene may be a substituted or unsubstituted 3 to 8 membered heterocycloalkylene.

Each substituted or unsubstituted alkyl may be a substituted or unsubstituted C₁-C₈ alkyl, each substituted or unsubstituted heteroalkyl may be a substituted or unsubstituted 2 to 8 membered heteroalkyl, each substituted or unsubstituted cycloalkyl may be a substituted or unsubstituted C₃-C₇ cycloalkyl, and/or each substituted or unsubstituted heterocycloalkyl may be a substituted or unsubstituted 3 to 7 membered heterocycloalkyl. Each substituted or unsubstituted alkylene may be a substituted or unsubstituted C₁-C₈ alkylene, each substituted or unsubstituted heteroalkylene may be a substituted or unsubstituted 2 to 8 membered heteroalkylene, each substituted or unsubstituted cycloalkylene may be a substituted or unsubstituted C₃-C₇ cycloalkylene, and/or each substituted or unsubstituted heterocycloalkylene may be a substituted or unsubstituted 3 to 7 membered heterocycloalkylene.

Certain compounds herein possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the compounds described herein. The compounds described herein do not include those which are known in art to be too unstable to synthesize and/or isolate. The compounds described herein also are meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds described herein may exist in tautomeric forms, and that all such tautomeric forms of the compounds may be considered within the scope of the compounds described herein.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds, generally recognized as stable by those skilled in the art, are within the scope of the compounds described herein.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the compounds described herein.

The compounds described herein may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds described herein, whether radioactive or not, are encompassed within the scope of the compounds described herein.

The symbol “ [symbol] ” denotes the point of attachment of a chemical moiety to the remainder of a molecule or chemical formula.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman decimal symbol may be used to distinguish each appearance of that particular R group. For example, where multiple R¹³ substituents are present, each R¹³ substituent may be distinguished as R^{13.1}, R^{13.2}, R^{13.3}, R^{13.4}, etc., wherein each of R^{13.1}, R^{13.2}, R^{13.3C}, R^{13.4}, etc. is defined within the scope of the definition of R¹³ and optionally differently.

Description of compounds described herein is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating”, or “treatment” refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment of symptoms can be based on objective or subjective parameters; including the results of a physical examination, neuropsychiatric exams, and/or a psychiatric evaluation. The term “treating” and conjugations thereof, include prevention of an injury, pathology, condition, or disease.

An “effective amount” is an amount sufficient to accomplish a stated purpose (e.g. achieve the effect for which it is administered, treat a disease, reduce enzyme activity, or reduce one or more symptoms of a disease or condition). An example of an “effective amount” is an amount sufficient to contribute to the treatment, prevention, or reduction of a symptom or symptoms of a disease, which herein is referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Pharmaceutically acceptable excipient” and “pharmaceutically acceptable carrier” refer to a substance that aids the administration of an active agent to and absorption by a subject and can be included in the compositions of the compounds described herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds described herein. One of skill in the art will recognize that other pharmaceutical excipients are useful in combination with the compounds described herein.

Pharmaceutical compositions may include compositions wherein the active ingredient (e.g. compounds described herein) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds described herein. Adjustment and manipulation of established dosages (e.g., frequency and duration) are well within the ability of those skilled in the art.

The compounds and complexes described herein can be used in combination with one another, with other active drugs known to be useful in treating a disease (e.g. anti-cancer agents) or with adjunctive agents that may not be effective alone, but may contribute to the efficacy of the active agent.

In a first aspect, there is provided a nanoparticle composition including a responsive nanoparticle. The responsive nanoparticle includes an enzyme responsive peptide-polymer amphiphile (PPA) and an enzymatically cleavable moiety, wherein the enzymatically cleavable moiety is cleaved by an enzyme which is upregulated in heart tissue post-myocardial infarction.

The term “responsive nanoparticle” and the like refer to a nanoparticle (e.g., a spherical micellar nanoparticle as disclosed herein) which can undergo a morphological transition from a discrete material (e.g., spherical-shaped micellar nanoparticle) to a network-like assemblies in response to enzymes upregulated in infarcted myocardium.

In embodiments, the enzyme upregulated in infarcted myocardium is a protease or a nuclease. In embodiments, the enzyme is a matrix metalloproteinase (MMP). In embodiments, the MMP is MMP-2 or MMP-9, as known in the art.

In embodiments, the PPA includes a polymer, e.g., a polymer as disclosed herein. In embodiments, the PPA further includes a drug or small molecule conjugated to said PPA. In embodiments, the PPA further includes a peptide or protein conjugated to the PPA. In embodiments, the PPA further includes a contrast agent conjugated to the PPA.

In another aspect, there is provided a nanoparticle including a peptide-polymer amphiphile (PPA) as disclosed herein, wherein the nanoparticle undergoes a morphology switch from nanoparticle to aggregate assembly in response to contact with infarcted myocardium.

In embodiments, the morphology switch is triggered by a protease or nuclease. In embodiments, the protease or nuclease cleaves the enzymatically cleavable moiety. In embodiments, the morphology switch is triggered by a matrix metalloproteinase (MMP).

In embodiments, the PPA includes a polymer. In embodiments, the PPA includes a drug or small molecule conjugated to the PPA. In embodiments, the PPA includes a peptide or protein conjugated to the PPA. In embodiments, the PPA includes a nucleic acid conjugated to the PPA. In embodiments, the PPA includes a contrast agent conjugated to the PPA.

In another aspect, there is provided a polymer including an enzymatic cleavable moiety, wherein the enzyme cleavable moiety is cleaved by an enzyme unregulated in heart tissue post-myocardial infarction.

In embodiments, the polymer self-assembles into an amphiphilic aggregate in infarcted myocardium. The terms “micron scale aggregate,” “amphiphilic aggregate” and “aggregate assembly” are used herein synonymously in the context of aggregations of nanoparticles and polymers disclosed herein which can undergo aggregation in response to contact with infarcted myocardium.

In embodiments, the enzyme is a protease or nuclease. In embodiments, the enzyme is a matrix metalloproteinase (MMP).

In embodiments, the polymer further includes a drug or small molecule conjugated to the polymer. In embodiments, the polymer further includes a peptide or protein conjugated to the polymer. In embodiments, the polymer further includes a nucleic acid conjugated to the polymer. In embodiments, the polymer further includes a contrast agent conjugated to the polymer.

Methods.

In another aspect, there is provided a method for treating myocardial infarction in a subject. The method includes administering to a subject in need thereof a nanoparticle as disclosed herein, wherein the administering is via intravenous, intracoronary, or intramyocardial delivery.

In embodiments, the nanoparticle undergoes a morphology switch from nanoparticle to micron scale aggregate in infarcted myocardium.

In embodiments, the nanoparticle delivers a drug or small molecule to the infarcted myocardium. In embodiments, the nanoparticle delivers a peptide or protein to the infarcted myocardium. In embodiments, the nanoparticle delivers a nucleic acid to the infarcted myocardium.

In another aspect, there is provided a method for treating myocardial infarction in a subject. The method includes administering to a subject in need thereof a polymer as disclosed herein, wherein the administering is via intravenous, intracoronary, or intramyocardial delivery.

In embodiments, the polymer self-assembles into an amphiphilic aggregate in infarcted myocardium.

In embodiments, the polymer delivers a drug or small molecule to heart tissue post-myocardial infarction. In embodiments, the polymer delivers a peptide or protein to heart tissue post-myocardial infarction. In embodiments, the polymer delivers a nucleic acid to heart tissue post-myocardial infarction.

Pharmaceutical Compositions.

In another aspect, there is provided a pharmaceutical composition including a nanoparticle composition, a nanoparticle, or a polymer as disclosed herein in combination with a pharmaceutically acceptable excipient.

### Embodiment P1

A nanoparticle composition comprising a responsive nanoparticle, said responsive nanoparticle comprising an enzyme responsive peptide-polymer amphiphile (PPA) and an enzymatically cleavable moiety, wherein said enzymatically cleavable moiety is cleaved by an enzyme which is upregulated in heart tissue post-myocardial infarction.

### Embodiment P2

The nanoparticle composition of Embodiment P1, wherein said enzyme is a protease or a nuclease.

### Embodiment P3

The nanoparticle composition of Embodiment P1, wherein said enzyme is a matrix metalloproteinase (MMP).

### Embodiment P4

The nanoparticle composition of Embodiment P1, wherein said PPA comprises a polymer.

### Embodiment P5

The nanoparticle composition of Embodiment P1, wherein said PPA further comprises a drug or small molecule conjugated to said PPA.

### Embodiment P6

The nanoparticle composition of Embodiment P1, wherein said PPA further comprises a peptide or protein conjugated to said PPA.

### Embodiment P7

The nanoparticle composition of Embodiment P1, wherein said PPA further comprises a nucleic acid conjugated to said PPA.

### Embodiment P8

The nanoparticle composition of Embodiment P1, where said PPA further comprises a contrast agent conjugated to said PPA.

### Embodiment P9

A nanoparticle comprising a peptide-polymer amphiphile (PPA) and an enzymatically cleavable moiety according to any one of Embodiments P1 to P8, wherein said nanoparticle undergoes a morphology switch from nanoparticle to aggregate assembly in response to contact with infarcted myocardium.

### Embodiment P10

The nanoparticle of Embodiment P9, wherein said morphology switch is triggered by a protease or nuclease.

### Embodiment P11

The nanoparticle of Embodiment P9, wherein said morphology switch is triggered by a matrix metalloproteinase (MMP).

### Embodiment P12

The nanoparticle of Embodiment P9, wherein said PPA comprises a polymer.

### Embodiment P13

The nanoparticle of Embodiment P9, wherein said PPA comprises a drug or small molecule conjugated to said PPA.

### Embodiment P14

The nanoparticle of Embodiment P9, wherein said PPA comprises a peptide or protein conjugated to said PPA.

### Embodiment P15

The nanoparticle of Embodiment P9, wherein said PPA comprises a nucleic acid conjugated to said PPA.

### Embodiment P16

The nanoparticle of Embodiment P9, wherein said PPA comprises a contrast agent conjugated to said PPA.

### Embodiment P17

A polymer comprising an enzymatic cleavable moiety, wherein the enzyme cleavable moiety is cleaved by an enzyme unregulated in heart tissue post-myocardial infarction.

### Embodiment P18

The polymer of Embodiment P17, wherein said polymer self-assembles into an amphiphilic aggregate in infarcted myocardium.

### Embodiment P19

The polymer of Embodiment P17, wherein the enzyme is a protease or nuclease.

### Embodiment P20

The polymer of Embodiment P17, wherein the enzyme is a matrix metalloproteinase (MMP).

### Embodiment P21

The polymer of Embodiment P17, further comprising a drug or small molecule conjugated to said polymer.

### Embodiment P22

The polymer of Embodiment P17, further comprising a peptide or protein conjugated to said polymer.

### Embodiment P23

The polymer of Embodiment P17, further comprising a nucleic acid conjugated to said polymer.

### Embodiment P24

The polymer of Embodiment P17, further comprising a contrast agent conjugated to said polymer.

### Embodiment P25

A method for treating myocardial infarction in a subject, said method comprising administering to a subject in need thereof a nanoparticle according to any one of Embodiments P9 to P16, said administering via intravenous, intracoronary, or intramyocardial delivery.

### Embodiment P26

The method of Embodiment P25, wherein said nanoparticle undergoes a morphology switch from nanoparticle to micron scale aggregate in infarcted myocardium.

### Embodiment P27

The method of Embodiment P25, wherein said nanoparticle delivers a drug or small molecule to said infarcted myocardium.

### Embodiment P28

The method of Embodiment P25 wherein said nanoparticle delivers a peptide or protein to said infarcted myocardium.

### Embodiment P29

The method of Embodiment P25 wherein said nanoparticle delivers a nucleic acid to said infarcted myocardium.

### Embodiment P30

A method for treating myocardial infarction in a subject, said method comprising administering to a subject in need thereof a polymer according to any one of Embodiments P17 to P24, said administering via intravenous, intracoronary, or intramyocardial delivery.

### Embodiment P31

The method of Embodiment P30, wherein said polymer self-assembles into an amphiphilic aggregate in infarcted myocardium

### Embodiment P32

The method of Embodiment P30, wherein said polymer delivers a drug or small molecule to heart tissue post-myocardial infarction.

### Embodiment P33

The method of Embodiment P30, wherein said polymer delivers a peptide or protein to heart tissue post-myocardial infarction.

### Embodiment P34

The method of Embodiment P30, wherein said polymer delivers a nucleic acid to heart tissue post-myocardial infarction.

### Embodiment P35

A nanoparticle with an enzymatic cleavable moiety wherein the enzyme cleavable moiety is cleaved by an enzyme present in myocardial infarction.

### Embodiment P36

Composition in Embodiment P35 wherein the enzyme is a protease or nuclease.

### Embodiment P37

Composition in Embodiment P35 wherein the enzyme is a matrix metalloproteinase (MMP).

### Embodiment P38

Composition in Embodiment P35 where the nanoparticle is composed of a polymer.

### Embodiment P39

Composition in Embodiment P35 where a drug or small molecule is conjugated to the nanoparticle.

### Embodiment P40

Composition in Embodiment P35 where a peptide or protein is conjugated to the nanoparticle.

### Embodiment P41

Composition in Embodiment P1 where a nucleic acid is conjugated to the nanoparticle.

### Embodiment P42

Composition in Embodiment P1 where a contrast agent is conjugated to the nanoparticle.

### Embodiment P43

A nanoparticle that undergoes a morphology switch from nanoparticle to micron scale aggregate in infarcted myocardium.

### Embodiment P44

Composition in Embodiment P43 wherein the morphology switch is triggered by a protease or nuclease.

### Embodiment P45

Composition in Embodiment P43 wherein the morphology switch is triggered by a MMP.

### Embodiment P46

Composition in Embodiment P43 where the nanoparticle is composed of a polymer.

### Embodiment P47

Composition in Embodiment P43 where a drug or small molecule is conjugated to the nanoparticle.

### Embodiment P48

Composition in Embodiment P43 where a peptide or protein is conjugated to the nanoparticle.

### Embodiment P49

Composition in Embodiment P43 where a nucleic acid is conjugated to the nanoparticle.

### Embodiment P50

Composition in Embodiment P43 where a contrast agent is conjugated to the nanoparticle.

### Embodiment P51

A polymer with an enzymatic cleavable moiety wherein the enzyme cleavable moiety is cleaved by an enzyme present in myocardial infarction.

### Embodiment P52

Composition of Embodiment P51 where the polymer self-assembles into an amphiphilic aggregate in infarcted myocardium.

### Embodiment P53

Composition in Embodiment P51 wherein the enzyme is a protease or nuclease.

### Embodiment P54

Composition in Embodiment P51 wherein the enzyme is a matrix metalloproteinase (MMP).

### Embodiment P55

Composition in Embodiment P51 where a drug or small molecule is conjugated to the polymer.

### Embodiment P56

Composition in Embodiment P51 where a peptide or protein is conjugated to the polymer.

### Embodiment P57

Composition in Embodiment P51 where a nucleic acid is conjugated to the polymer.

### Embodiment P58

Composition in Embodiment P51 where a contrast agent is conjugated to the polymer.

### Embodiment P59

A method for treating myocardial infarction in a subject comprising administering a nanoparticle via intravenous, intracoronary, or intramyocardial delivery wherein the nanoparticle contains an enzyme cleavable moiety that is cleaved at the site of myocardial infarction.

### Embodiment P60

Method of Embodiment P59 where the nanoparticle undergoes a morphology switch from nanoparticle to micron scale aggregate in infarcted myocardium.

### Embodiment P61

Method of Embodiment P59 where the nanoparticle delivers a drug or small molecule.

### Embodiment P62

Method of Embodiment P59 where the nanoparticle delivers a peptide or protein.

### Embodiment P63

Method of Embodiment P59 where the nanoparticle delivers a nucleic acid.

### Embodiment P64

A method for treating myocardial infarction in a subject comprising administering a polymer via intravenous, intracoronary, or intramyocardial delivery wherein the polymer contains an enzyme cleavable moiety that is cleaved at the site of myocardial infarction.

### Embodiment P65

Method of Embodiment P64 where the polymer self-assembles into an amphiphilic aggregate in infarcted myocardium.

### Embodiment P66

Method of Embodiment P64 where the polymer delivers a drug or small molecule.

### Embodiment P67

Method of Embodiment P64 where the polymer delivers a peptide or protein.

### Embodiment P68

Method of Embodiment P30 where the polymer delivers a nucleic acid.

### Additional Embodiments

### Embodiment 1

A block copolymer comprising a first block of hydrophobic polymerized monomers and a second block of hydrophilic polymerized monomers, wherein the first block of hydrophobic polymerized monomers comprise a hydrophobic moiety covalently attached to each first block monomer backbone moiety within said first block of hydrophobic polymerized monomers, wherein each hydrophobic moiety is optionally different; and the second block of hydrophilic polymerized monomers comprise a hydrophilic moiety covalently attached to each second block monomer backbone moiety within said second block of hydrophilic polymerized monomers, wherein each hydrophilic moiety is optionally different, and wherein at least one of said hydrophilic moieties comprises an MMP-9 or MMP-2 cleavable amino acid sequence.

### Embodiment 2

The block copolymer of Embodiment 1, wherein each of said hydrophobic moieties are the same.

### Embodiment 3

The block copolymer of Embodiment 1, wherein each of said hydrophobic moieties is either: (a) a first hydrophobic moiety comprising a hydrophobic drug moiety, targeting moiety, or a detectable moiety or (b) a second hydrophobic moiety not comprising a hydrophobic drug moiety.

### Embodiment 4

The block copolymer of one of Embodiments 1 to 3, wherein each of said hydrophilic moieties is either: (a) a first hydrophilic moiety comprising an MMP-9 or MMP-2 cleavable amino acid sequence or (b) a second hydrophilic moiety not comprising an amino acid sequence.

### Embodiment 5

The block copolymer of Embodiment 1 of the formula:

R¹-L¹-[(A(-L²-R²))_{z1}—(B(-L³-R³))_{z2}-(A(-L²-R²))_{z3}]_{z4}—[(C(-L⁴-R⁴))_{z5}-(D(-L⁵-R⁵))_{z6}—(C(-L⁴-R⁴))_{z7})]_{z8}-L⁶-R⁶

wherein
[(A(-L²-R²))_{z1}—(B(-L³-R³))_{z2}-(A(-L²-R²))_{z3}]_{z4} is the first block of hydrophobic polymerized monomers;
[(C(-L⁴-R⁴))_{z5}-(D(-L⁵-R⁵))_{z6}—(C(-L⁴-R⁴))_{z7})]_{z8} is the second block of hydrophilic polymerized monomers;
A and B are a first block monomer backbone moiety;
C and D are a second block monomer backbone moiety;
z1, z3, z5 and z7 are independently integers from 0 to 100;
z2, z4, z6 and z8 are independently integers from 1 to 100
L¹ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a hydrophobic drug moiety, a targeting moiety or a detectable moiety;
L²-R² is a hydrophobic moiety, wherein
   L² is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
   R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a hydrophobic drug moiety, a targeting moiety or a detectable moiety;
L³-R³ is a hydrophobic moiety, wherein
   L³ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
   R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴-R⁴ is a hydrophilic moiety, wherein
   L⁴ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
   R⁴ is hydrogen, —OH, —SH, —NH₂, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a hydrophilic drug moiety, a targeting moiety or a detectable moiety; and
L⁵-R⁵ is a hydrophilic moiety, wherein
   L⁵ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
   R⁵ is an amino acid sequence comprising said MMP-9 or MMP-2 cleavable amino acid sequence;
L⁶ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R⁶ is hydrogen, —OH, —SH, —NH₂, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a hydrophilic drug moiety, a targeting moiety or a detectable moiety.

### Embodiment 6

The block copolymer of Embodiment 5, wherein said polymer comprises a single drug moiety.

### Embodiment 7

The block copolymer of Embodiment 5 or 6, wherein z1 and z3 are 0.

### Embodiment 8

The block copolymer of one of Embodiments 5 to 7, wherein z5 and z7 are 0.

### Embodiment 9

The block copolymer of one of Embodiments 5 to 8, wherein z4 and z8 are 1.

### Embodiment 10

The block copolymer of one of Embodiments 5 to 9, wherein z2 is an integer from 10-35 and z6 is 2 or 4.

### Embodiment 11

The block copolymer of one of Embodiments 5 to 9, wherein z2 is an integer from 10-35, z5 is an integer from 1 to 6, z6 is 3 or 4.

### Embodiment 12

The block copolymer of one of Embodiments 5 to 11, wherein (A(-L²-R²))_{z1} has the formula (-L^{1A}-A¹(-L²-R²)-L^{2A}-)_{z1}, wherein
L^{1A} and L^{2A} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
A¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

### Embodiment 13

The block copolymer of one of Embodiments 5 to 11, wherein (A(-L²-R²))_{z1} has the formula:

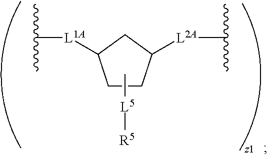

wherein
L^{1A} and L^{2A} are independently a bond, —O—, —S—, —NH—, —C(O)—, —C(O)O—, —C(O)NH—, substituted or unsubstituted alkylene, substituted or unsubstituted heteroalkylene.

### Embodiment 14

The block copolymer of one of Embodiments 5 to 13, wherein (A(-L²-R²))_{z3} has the formula (-L^{1A}-A¹(-L²-R²)-L^{2A}-)_{z3} wherein
L^{1A} and L^{2A} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
A¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

### Embodiment 15

The block copolymer of one of Embodiments 5 to 13, wherein (A(-L²-R²))_{z3} has the formula:

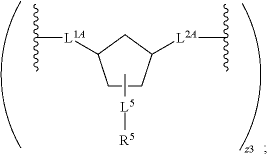

wherein
L^{1A} and L^{2A} are independently a bond, —O—, —S—, —NH—, —C(O)—, —C(O)O—, —C(O)NH—, substituted or unsubstituted alkylene, substituted or unsubstituted heteroalkylene.

### Embodiment 16

The block copolymer of one of Embodiments 5 to 15, wherein (B(-L³-R³))_{z2} has the formula (-L^{1B}-B¹(-L³-R³)-L^{2B}-)_{z2}, wherein
L^{1B} and L^{2B} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
B¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

### Embodiment 17

The block copolymer of one of Embodiments 5 to 15, wherein (B(-L³-R³))_{z2} has the formula:

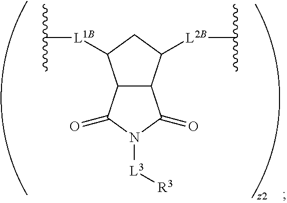

wherein
L^{1B} and L^{2B} are independently a bond, —O—, —S—, —NH—, —C(O)—, —C(O)O—, —C(O)NH—, substituted or unsubstituted alkylene, substituted or unsubstituted heteroalkylene.

### Embodiment 18

The block copolymer of one of Embodiments 5 to 17, wherein (C(-L⁴-R⁴))_{z5} has the formula (-L^{1C}-C¹(-L⁴-R⁴)-L^{2C}-)_{z5}, wherein
L^{1C} and L^{2C} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
C¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

### Embodiment 19

The block copolymer of one of Embodiments 5 to 17, wherein (C(-L⁴-R⁴))_{z5} has the formula:

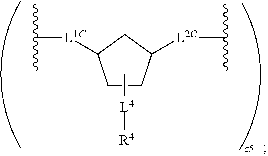

wherein
L^{1C} and L^{2C} are independently a bond, —O—, —S—, —NH—, —C(O)—, —C(O)O—, —C(O)NH—, substituted or unsubstituted alkylene, substituted or unsubstituted heteroalkylene.

### Embodiment 20

The block copolymer of one of Embodiments 5 to 19, wherein (C(-L⁴-R⁴))_{z7} has the formula (-L^{1C}-C¹(-L⁴-R⁴)-L^{2C}-)_{z7}, wherein
L^{1C} and L^{2C} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
C¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

### Embodiment 21

The block copolymer of one of Embodiments 5 to 20, wherein (C(-L⁴-R⁴))_{z7} has the formula:

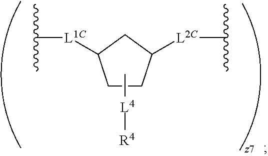

wherein
L^{1C} and L^{2C} are independently a bond, —O—, —S—, —NH—, —C(O)—, —C(O)O—, —C(O)NH—, substituted or unsubstituted alkylene, substituted or unsubstituted heteroalkylene.

### Embodiment 22

The block copolymer of one of Embodiments 5 to 21, wherein L⁴-R⁴ is —C(O)OH.

### Embodiment 23

The block copolymer of one of Embodiments 5 to 22, wherein L³-R³ is

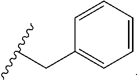 [SMILES: *Cc1ccccc1]

### Embodiment 24

The block copolymer of one of Embodiments 5 to 23, wherein (D(-L⁵-R⁵))_{z6} has the formula (-L^{1D}-D¹(-L⁵-R⁵)-L^{2D}-)_{z6}, wherein
L^{1D} and L^{2D} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
D¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

### Embodiment 25

The block copolymer of one of Embodiments 5 to 23, wherein (D(-L⁵-R⁵))_{z6} has the formula:

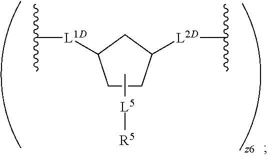

wherein
L^{1D} and L^{2D} are independently a bond, —O—, —S—, —NH—, —C(O)—, —C(O)O—, —C(O)NH—, substituted or unsubstituted alkylene, substituted or unsubstituted heteroalkylene.

### Embodiment 26

The block copolymer of one of Embodiments 5 to 25, wherein L⁵ is a bond, —O—, —S—, —NH—, —C(O)—, —C(O)O—, —C(O)NH—.

### Embodiment 27

The block copolymer of one of Embodiments 5 to 26, wherein (D(-L⁵-R⁵))_{z6} has the formula:

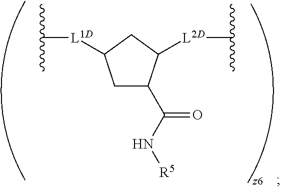

wherein
L¹D and L^{2D} are independently a bond, —O—, —S—, —NH—, —C(O)—, —C(O)O—, —C(O)NH—, substituted or unsubstituted alkylene, substituted or unsubstituted heteroalkylene.

### Embodiment 28

The block copolymer of one of Embodiments 5 to 27, wherein L⁶ is

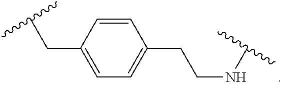 [SMILES: *Cc1ccc(CCN*)cc1]

### Embodiment 29

The block copolymer of one of Embodiments 5 to 28, wherein L^{1A}, L^{2A}, L^{1B}, L^{2B}, L^{1C}, L^{2C}, L^{1D}, L^{2D} are independently a bond or a substituted or unsubstituted alkylene.

### Embodiment 30

The block copolymer of one of Embodiments 5 to 28, wherein L^{1A}, L^{2A}, L^{1B}, L^{2B}, L^{1C}, L^{2C}, L^{1D}, L^{2D} are independently a bond or a substituted or unsubstituted alkenylene.

### Embodiment 31

A block copolymer including a first block of hydrophobic polymerized monomers and a second block of hydrophilic polymerized monomers, wherein the first block of hydrophobic polymerized monomers include a hydrophobic moiety covalently attached to each first block monomer backbone moiety within the first block of hydrophobic polymerized monomers, wherein each hydrophobic moiety is optionally different; and the second block of hydrophilic polymerized monomers include a hydrophilic moiety covalently attached to each second block monomer backbone moiety within the second block of hydrophilic polymerized monomers, wherein each hydrophilic moiety is optionally different, and wherein at least one of the hydrophilic moieties includes an inflammatory protease cleavable amino acid sequence.

### Embodiment 32

The block copolymer of Embodiment 31, wherein the inflammatory protease cleavable amino acid sequence is a cardiovascular inflammatory protease cleavable amino acid sequence.

### Embodiment 33

The block copolymer of Embodiment 31, wherein the inflammatory protease cleavable amino acid sequence is a myocardial inflammatory protease cleavable amino acid sequence.

### Embodiment 34

A method of forming a polymeric aggregate, the method including: contacting the block copolymer of one of Embodiments 1 to 33 with an MMP-2 or MMP-9; and allowing the MMP-2 or MMP-9 to cleave the MMP-2 or MMP-9 cleavable amino acid sequence, respectively, thereby forming a polymeric aggregate.

### Embodiment 35

A polymeric micelle including a plurality of the block copolymer of one of Embodiments 1 to 33, the polymeric micelle including a hydrophobic core including the first block of hydrophobic polymerized monomers and a hydrophilic shell including the second block of hydrophilic polymerized monomers.

### Embodiment 36

An aqueous pharmaceutical composition including the polymeric micelle of Embodiment 35 and a pharmaceutically acceptable excipient.

### Embodiment 37

The pharmaceutical composition of Embodiment 36, wherein the pharmaceutical composition is a parental dosage form.

### Embodiment 38

The pharmaceutical composition of Embodiment 36, wherein the pharmaceutical composition is an intravenous dosage form.

### Embodiment 39

The pharmaceutical composition of Embodiment 36 or 38, wherein the pharmaceutical composition is isotonic and has a pH from about 3.5 to about 6.2.

### Embodiment 40

A method of treating a myocardial infarction in a subject in need thereof, the method including administering to the subject a therapeutically effective amount of the block copolymer of one of Embodiments 1 to 33, the polymeric micelle of Embodiment 35, or the pharmaceutical composition of one of Embodiments 36 to 39.

### Embodiment 41

The method of Embodiment 40, wherein the polymeric micelle or the pharmaceutical composition is administered intravenously.

### Embodiment 42

The method of Embodiment 40, wherein the polymeric micelle or the pharmaceutical composition is administered via intracoronary delivery.

### Embodiment 43

A method of forming a polymeric aggregate, the method including: contacting the polymeric micelle of Embodiment 35 with an MMP-2 or MMP-9; and allowing the MMP-2 or MMP-9 to cleave the MMP-2 or MMP-9 cleavable amino acid sequence, respectively, thereby forming a polymeric aggregate.

### Embodiment 44

A method of treating heart failure in a subject in need thereof, the method including administering to the subject a therapeutically effective amount of the block copolymer of one of Embodiments 1 to 33, the polymeric micelle of Embodiment 35, or the pharmaceutical composition of one of Embodiments 36 to 39.

### Embodiment 45

A method of performing cardiovascular surgery in a subject in need thereof, the method including: performing a surgical procedure; and administering to the subject a therapeutically effective amount of the block copolymer of one of Embodiments 1 to 33, the polymeric micelle of Embodiment 35, or the pharmaceutical composition of one of Embodiments 36 to 39.

### Embodiment 46

The method of Embodiment 45, wherein the surgical procedure is an angioplasty or stent placement.

1. A block copolymer comprising a first block of hydrophobic polymerized monomers and a second block of hydrophilic polymerized monomers, wherein:
(i) the first block of hydrophobic polymerized monomers comprises a hydrophobic moiety covalently attached to each first block monomer backbone moiety within said first block of hydrophobic polymerized monomers, wherein each hydrophobic moiety is optionally different; and
(ii) the second block of hydrophilic polymerized monomers comprises a hydrophilic moiety covalently attached to each second block monomer backbone moiety within said second block of hydrophilic polymerized monomers, wherein each hydrophilic moiety is optionally different, and wherein at least one of said hydrophilic moieties comprises an MMP-9 or MMP-2 cleavable amino acid sequence.

2-4. (canceled)

5. The block copolymer of claim 1 of the formula:
R¹-L¹-[(A(-L²-R²))_{z1}—(B(-L³-R³))_{z2}-(A(-L²-R²))_{z3}]_{z4}—[(C(-L⁴-R⁴))_{z5}-(D(-L⁵-R⁵))_{z6}—(C(-L⁴-R⁴))_{z7})]_{z8}-L⁶-R⁶
wherein
[(A(-L²-R²))_{z1}—(B(-L³-R³))_{z2}-(A(-L²-R²))_{z3}]_{z4} is the first block of hydrophobic polymerized monomers;
[(C(-L⁴-R⁴))_{z5}-(D(-L⁵-R⁵))_{z6}—(C(-L⁴-R⁴))_{z7})]_{z8} is the second block of hydrophilic polymerized monomers;
A and B are a first block monomer backbone moiety;
C and D are a second block monomer backbone moiety;
z1, z3, z5 and z7 are independently integers from 0 to 100;
z2, z4, z6 and z8 are independently integers from 1 to 100
L¹ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a hydrophobic drug moiety, a targeting moiety or a detectable moiety;
L²-R² is a hydrophobic moiety, wherein
L² is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a hydrophobic drug moiety, a targeting moiety or a detectable moiety;
L³-R³ is a hydrophobic moiety, wherein
L³ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴-R⁴ is a hydrophilic moiety, wherein
L⁴ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁴ is hydrogen, —OH, —SH, —NH₂, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a hydrophilic drug moiety, a targeting moiety or a detectable moiety; and
L⁵-R⁵ is a hydrophilic moiety, wherein
L⁵ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R⁵ is an amino acid sequence comprising said MMP-9 or MMP-2 cleavable amino acid sequence;
L⁶ is a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R⁶ is hydrogen, —OH, —SH, —NH₂, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a hydrophilic drug moiety, a targeting moiety or a detectable moiety.

6-11. (canceled)

12. The block copolymer of claim 5, wherein (A(-L²-R²))_{z1} has the formula (-L^{1A}-A¹(-L²-R²)-L^{2A}-)_{z1}, wherein
L^{1A} and L^{2A} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
A¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

13. (canceled)

14. The block copolymer of claim 5, wherein (A(-L²-R²))_{z3} has the formula (-L^{1A}-A¹(-L²-R²)-L^{2A}-)_{z3}, wherein
L^{1A} and L^{2A} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
A¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

15. (canceled)

16. The block copolymer of claim 5, wherein (B(-L³-R³))_{z2} has the formula (-L^{1B}-B¹(-L³-R³)-L^{2B}-)_{z2}, wherein
L^{1B} and L^{2B} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
B¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

17. (canceled)

18. The block copolymer of claim 5, wherein (C(-L⁴-R⁴))_{z5} has the formula (-L^{1C}-C¹(-L⁴-R⁴)-L^{2C}-)_{z5}, wherein
L^{1C} and L^{2C} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
C¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

19. (canceled)

20. The block copolymer of claim 5, wherein (C(-L⁴-R⁴))_{z7} has the formula (-L^{1C}-C¹(-L⁴-R⁴)-L^{2C}-)_{z7}, wherein
L^{1C} and L^{2C} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
C¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

21-23. (canceled)

24. The block copolymer of claim 5, wherein (D(-L⁵-R⁵))_{z6} has the formula (-L^{1D}-D¹(-L⁵-R⁵)-L^{2D}-)_{z6}, wherein
L^{1D} and L^{2D} are independently a bond, —O—, —S—, —NH—, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
D¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

25. (canceled)

26. (canceled)

27. The block copolymer of claim 5, wherein (D(-L⁵-R⁵))_{z6} has the formula: 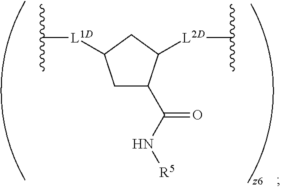
wherein
L^{1D} and L^{2D} are independently a bond, —O—, —S—, —NH—, —C(O)—, —C(O)O—, —C(O)NH—, substituted or unsubstituted alkylene, substituted or unsubstituted heteroalkylene.

28-30. (canceled)

31. A block copolymer comprising a first block of hydrophobic polymerized monomers and a second block of hydrophilic polymerized monomers, wherein:
i) the first block of hydrophobic polymerized monomers comprises a hydrophobic moiety covalently attached to each first block monomer backbone moiety within said first block of hydrophobic polymerized monomers, wherein each hydrophobic moiety is optionally different; and
ii) the second block of hydrophilic polymerized monomers comprises a hydrophilic moiety covalently attached to each second block monomer backbone moiety within said second block of hydrophilic polymerized monomers, wherein each hydrophilic moiety is optionally different, and wherein at least one of said hydrophilic moieties comprises an inflammatory protease cleavable amino acid sequence.

32. (canceled)

33. (canceled)

34. A method of forming a polymeric aggregate, the method comprising:
(i) contacting the block copolymer of claim 1 with an MMP-2 or MMP-9; and
(ii) allowing said MMP-2 or MMP-9 to cleave said MMP-2 or MMP-9 cleavable amino acid sequence, respectively, thereby forming a polymeric aggregate.

35. A polymeric micelle comprising a plurality of the block copolymer of claim 1, said polymeric micelle comprising a hydrophobic core comprising said first block of hydrophobic polymerized monomers and a hydrophilic shell comprising said second block of hydrophilic polymerized monomers.

36. An aqueous pharmaceutical composition comprising the polymeric micelle of claim 35 and a pharmaceutically acceptable excipient.

37-39. (canceled)

40. A method of treating a myocardial infarction in a subject in need thereof, said method comprising administering to said subject a therapeutically effective amount of the block copolymer of claim 1.

41. (canceled)

42. (canceled)

43. A method of forming a polymeric aggregate, the method comprising:
i) contacting the polymeric micelle of claim 35 with an MMP-2 or MMP-9; and
ii) allowing said MMP-2 or MMP-9 to cleave said MMP-2 or MMP-9 cleavable amino acid sequence, respectively, thereby forming a polymeric aggregate.

44. A method of treating heart failure in a subject in need thereof, said method comprising administering to said subject a therapeutically effective amount of the block copolymer of claim 1.

45. A method of performing cardiovascular surgery in a subject in need thereof, said method comprising:
i) performing a surgical procedure; and
ii) administering to said subject a therapeutically effective amount of the block copolymer of claim 1.

46. (canceled)

47. A method of treating a myocardial infarction in a subject in need thereof, said method comprising administering to said subject a therapeutically effective amount of the polymeric micelle of claim 35.

48. (canceled)

49. (canceled)

50. A method of treating heart failure in a subject in need thereof, said method comprising administering to said subject a therapeutically effective amount of the polymeric micelle of claim 35.

51. A method of performing cardiovascular surgery in a subject in need thereof, said method comprising:
i) performing a surgical procedure; and
ii) administering to said subject a therapeutically effective amount of the polymeric micelle of claim 35.

52. (canceled)
